(19) United States
(12) Patent Application Publication
(10) Pub. No.: US 2005/0239064 A1
(43) Pub. Date: Oct. 27, 2005

(54) Methods and compositions based on protein interactions with mastermind

(76) Inventors: Spyridon Artavanis-Tsakonas, Willard Road Brookline, MA (US); Robert J. Lake, Malden, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/481,596

(22) PCT Filed: Jun. 18, 2002
(86) PCT No.: PCT/US02/19189

§ 371 Date: Aug. 24, 2004

Related U.S. Application Data
(60) Provisional application No. 60/299,112, filed on Jun. 18, 2001.

(51) Int. Cl. C12Q 1/68; G01N 33/53; A61K 39/395; C12N 15/85; A61K 48/00

(52) U.S. Cl. 435/6; 435/7.1; 435/455; 514/44; 424/143.1

(57) ABSTRACT

The invention is directed to methods of modulating Notch signal transduction and to complexes of the protein Mastermind with proteins identified as interacting with Mastermind by a two-hybrid screen as well as a complex of Mastermind (Mam) with Mip1, or a complex of Mam with Mip30, or a complex of Mam with Mip6. Methods of screening the complexes for efficacy in treating and/or preventing certain diseases and disorders, particularly hyperproliferative and cancerous conditions are also provided. The invention includes nucleic acid and amino acid sequences of Mip30 or Mip6, as well as fragments and derivatives thereof.

### 1. FIELD OF THE INVENTION

The present invention is directed to modulating signal transduction.

### 2. BACKGROUND OF THE INVENTION

2.1 Notch Signal Transduction

Genetic and molecular studies have led to the identification of a group of genes which define distinct elements of the Notch signaling pathway. While the identification of these various elements has come exclusively from Drosophila using genetic tools as the initial guide, subsequent analyses have lead to the identification of homologous proteins in vertebrate species including humans. See, generally, Artavanis-Tsakonas et al., 1995, Science 268:225-232.

The Drosophila Notch gene encodes an ˜300 kD transmembrane protein that acts as a receptor in a cell-cell signaling mechanism controlling cell fate decisions throughout development (reviewed, e.g., in Artavanis-Tsakonas et al., 1995, Science 268:225-232). Closely related homologs of Drosophila Notch have been isolated from a number of vertebrate species, including humans, with multiple paralogs representing the single Drosophila gene in vertebrate genomes. The isolation of cDNA clones encoding the C-terminus of a human Notch paralog, originally termed h N, has been reported (Stifani et al., 1992, Nature Genetics 2:119-127). The encoded protein is designated human Notch2 because of its close relationship to the Notch2 proteins found in other species (Weinmaster et al., 1992, Development 116:931-941). The hallmark Notch2 structures are common to all the Notch-related proteins, including, in the extracellular domain, a stretch of 34 to 36 tandem Epidermal Growth Factor-like (EGF) repeats (fewer EGF repeats in Notch 3 and 4) and three Lin-12/Notch repeats (LN repeats), and, in the intracellular domain, 6 Ankyrin repeats and a PEST-containing region. Like Drosophila Notch and the related C. elegans genes lin-12 and glp-1 (Sternberg, 1993, Current Biology 3:763-765; Greenwald, 1994, Current Opinion in Genetics and Development 4:556-562), the vertebrate Notch homologs play a role in a variety of developmental processes by controlling cell fate decisions (reviewed, e.g., in Blaumueller and Artavanis-Tsakonas, 1997, Persp. on Dev. Neurobiol. 4:325-343). For further human Notch sequences, see International Publication WO 92/19734 and WO 99/04746.

The extracellular domain of Notch generally carries 36 Epidermal Growth Factor-like (EGF) repeats, two of which (repeats 11 and 12) have been implicated in interactions with the Notch ligands Serrate and Delta. Delta and Serrate are membrane bound ligands with EGF homologous extracellular domains, which interact physically with Notch on adjacent cells to trigger signaling.

Functional analyses involving the expression of truncated forms of the Notch receptor have indicated that receptor activation depends on the six cdc10/ankyrin repeats in the intracellular domain. Deltex and Suppressor of Hairless, whose over-expression results in an apparent activation of the pathway, associate with those repeats.

Deltex is a cytoplasmic protein which contains a ring zinc finger. Suppressor of Hairless on the other hand, is the Drosophila homolog of CBF1, a mammalian DNA binding protein involved in the Epstein-Barr virus-induced immortalization of B cells. It has been demonstrated that, at least in cultured cells, Suppressor of Hairless associates with the cdc10/ankyrin repeats in the cytoplasm and translocates into the nucleus upon the interaction of the Notch receptor with its ligand Delta on adjacent cells (Fortini and Artavanis, 1994, Cell 79:273-282). The association of Hairless, a novel nuclear protein, with Suppressor of Hairless has been documented using the yeast two hybrid system; therefore, it is believed that the involvement of Suppressor of Hairless in transcription is modulated by Hairless (Brou et al., 1994, Genes Dev. 8:2491; Knust et al. 1992, Genetics 129:803).

Finally, it is known that Notch signaling results in the activation of at least certain basic helix-loop-helix (bHLH) genes within the Enhancer of Split complex (Delidakis et al., 1991, Genetics 129:803).

The generality of the Notch pathway manifests itself at different levels. At the genetic level, many mutations exist which affect the development of a very broad spectrum of cell types in Drosophila. Knockout mutations in mice are embryonic lethals consistent with a fundamental role for Notch function (Swiatek et al., 1994, Genes Dev. 8:707). Mutations in the Notch pathway in the hematopoietic system in humans are associated with lymphoblastic leukemia (Ellison et al., 1991, Cell 66:649-661). Finally the expression of mutant forms of Notch in developing Xenopus embryos interferes profoundly with normal development (Coffman et al., 1993, Cell 73:659). Increased level of Notch expression is found in some malignant tissue in humans (International Publication WO 94/07474).

The expression patterns of Notch in the Drosophila embryo are complex and dynamic. The Notch protein is broadly expressed in the early embryo, and subsequently becomes restricted to uncommitted or proliferative groups of cells as development proceeds. In the adult, expression persists in the regenerating tissues of the ovaries and testes (reviewed in Fortini et al., 1993, Cell 75:1245-1247; Jan et al., 1993, Proc. Natl. Acad. Sci. USA 90:8305-8307; Sternberg, 1993, Curr. Biol. 3:763-765; Greenwald, 1994, Curr. Opin. Genet. Dev. 4:556-562; Artavanis-Tsakonas et al., 1995, Science 268:225-232). Studies of the expression of Notch1, one of three known vertebrate homologs of Notch, in zebrafish and Xenopus, have shown that the general patterns are similar; with Notch expression associated in general with non-terminally differentiated, proliferative cell populations. Tissues with high expression levels include the developing brain, eye and neural tube (Coffman et al., 1990, Science 249:1438-1441; Bierkamp et al., 1993, Mech. Dev. 43:87-100). While studies in mammals have shown the expression of the corresponding Notch homologs to begin later in development, the proteins are expressed in dynamic patterns in tissues undergoing cell fate determination or rapid proliferation (Weinmaster et al., 1991, Development 113:199-205; Reaume et al., 1992, Dev. Biol. 154:377-387; Stifani et al., 1992, Nature Genet. 2:119-127; Weinmaster et al., 1992, Development 116:931-941; Kopan et al., 1993, J. Cell Biol. 121:631-641; Lardelli et al., 1993, Exp. Cell Res. 204:364-372; Lardelli et al., 1994, Mech. Dev. 46:123-136; Henrique et al., 1995, Nature 375:787-790; Horvitz et al., 1991, Nature 351:535-541; Franco del Amo et al., 1992, Development 115:737-744). Among the tissues in which mammalian Notch homologs are first expressed are the pre-somitic mesoderm and the developing neuroepithelium of the embryo. In the pre-somitic mesoderm, expression of Notch1 is seen in all of the migrated mesoderm, and a particularly dense band is seen at the anterior edge of pre-somitic mesoderm. This expression has been shown to decrease once the somites have formed, indicating a role for Notch in the differentiation of somatic precursor cells (Reaume et al., 1992, Dev. Biol. 154:377-387; Horvitz et al., 1991, Nature 351:535-541). Similar expression patterns are seen for mouse Delta (Simske et al., 1995, Nature 375:142-145).

Within the developing mammalian nervous system, expression patterns of Notch homologs have been shown to be prominent in particular regions of the ventricular zone of the spinal cord, as well as in components of the peripheral nervous system, in an overlapping but non-identical pattern. Notch expression in the nervous system appears to be limited to regions of cellular proliferation, and is absent from nearby populations of recently differentiated cells (Weinmaster et al., 1991, Development 113:199-205; Reaume et al., 1992, Dev. Biol. 154:377-387; Weinmaster et al., 1992, Development 116:931-941; Kopan et al., 1993, J. Cell Biol. 121:631-641; Lardelli et al., 1993, Exp. Cell Res. 204:364-372; Lardelli et al., 1994, Mech. Dev. 46:123-136; Henrique et al., 1995, Nature 375:787-790; Horvitz et al., 1991, Nature 351:535-541). A rat Notch ligand is also expressed within the developing spinal cord, in distinct bands of the ventricular zone that overlap with the expression domains of the Notch genes. The spatio-temporal expression pattern of this ligand correlates well with the patterns of cells committing to spinal cord neuronal fates, which demonstrates the usefulness of Notch as a marker of populations of cells for neuronal fates (Henrique et al., 1995, Nature 375:787-790). This has also been suggested for vertebrate Delta homologues, whose expression domains also overlap with those of Notch1 (Larsson et al., 1994, Genomics 24:253-258; Fortini et al., 1993, Nature 365:555-557; Simske et al., 1995, Nature 375:142-145). In the cases of the Xenopus and chicken homologues, Delta is actually expressed only in scattered cells within the Notch1 expression domain, as would be expected from the lateral specification model, and these patterns “foreshadow” future patterns of neuronal differentiation (Larsson et al., 1994, Genomics 24:253-258; Fortini et al., 1993, Nature 365:555-557).

Other vertebrate studies of particular interest have focused on the expression of Notch homologs in developing sensory structures, including the retina, hair follicles and tooth buds. In the case of the Xenopus retina, Notch1 is expressed in the undifferentiated cells of the central marginal zone and central retina (Coffman et al., 1990, Science 249:1439-1441; Mango et al., 1991, Nature 352:811-815). Studies in the rat have also demonstrated an association of Notch1 with differentiating cells in the developing retina have been interpreted to suggest that Notch1 plays a role in successive cell fate choices in this tissue (Lyman et al., 1993, Proc. Natl. Acad. Sci. USA 90:10395-10399).

A detailed analysis of mouse Notch1 expression in the regenerating matrix cells of hair follicles was undertaken to examine the potential participation of Notch proteins in epithelial/mesenchymal inductive interactions (Franco del Amo et al., 1992, Development 115:737-744). Such a role had originally been suggested for Notch1 based on the its expression in rat whiskers and tooth buds (Weinmaster et al., 1991, Development 113:199-205). Notch1 expression was instead found to be limited to subsets of non-mitotic, differentiating cells that are not subject to epithelial/mesenchymal interactions, a finding that is consistent with Notch expression elsewhere.

Expression studies of Notch proteins in human tissue and cell lines have also been reported. The aberrant expression of a truncated Notch1 RNA in human T-cell leukemia results from a translocation with a breakpoint in Notch1 (Ellisen et al., 1991, Cell 66:649-661). A study of human Notch1 expression during hematopoiesis has suggested a role for Notch1 in the early differentiation of T-cell precursors (Mango et al., 1994, Development 120:2305-2315). Additional studies of human Notch1 and Notch2 expression have been performed on adult tissue sections including both normal and neoplastic cervical and colon tissue. Notch1 and Notch2 appear to be expressed in overlapping patterns in differentiating populations of cells within squamous epithelia of normal tissues that have been examined and are clearly not expressed in normal columnar epithelia, except in some of the precursor cells. Both proteins are expressed in neoplasias, in cases ranging from relatively benign squamous metaplasias to cancerous invasive adenocarcinomas in which columnar epithelia are replaced by these tumors (Mello et al., 1994, Cell 77:95-106).

Insight into the developmental role and the general nature of Notch signaling has emerged from studies with truncated, constitutively activated forms of Notch in several species. These recombinantly engineered Notch forms, which lack extracellular ligand-binding domains, resemble the naturally occurring oncogenic variants of mammalian Notch proteins and are constitutively activated using phenotypic criteria (Greenwald, 1994, Curr. Opin. Genet. Dev. 4:556; Fortini et al., 1993, Nature 365:555-557; Coffman et al., 1993, Cell 73:659-671; Struhl et al., 1993, Cell 69:1073; Rebay et al., 1993, Genes Dev. 7:1949; Kopan et al., 1994, Development 120:2385; Roehl et al., 1993, Nature 364:632).
Ubiquitous expression of activated Notch in the Drosophila embryo suppresses neuroblast segregation without impairing epidermal differentiation (Struhl et al., 1993, Cell 69:331; Rebay et al., 1993, Genes Dev. 7:1949).
Persistent expression of activated Notch in developing imaginal epithelia likewise results in an overproduction of epidermis at the expense of neural structures (Struhl et al., 1993, Cell 69:331).
Neuroblast segregation occurs in temporal waves that are delayed but not prevented by transient expression of activated Notch in the embryo (Struhl et al., 1993, Cell 69:331).
Transient expression in well-defined cells of the Drosophila eye imaginal disc causes the cells to ignore their normal inductive cues and to adopt alternative cell fates (Fortini et al., 1993, Nature 365:555-557).
Studies utilizing transient expression of activated Notch in either the Drosophila embryo or the eye disc indicate that once Notch signaling activity has subsided, cells may recover and differentiate properly or respond to later developmental cues (Fortini et al., 1993, Nature 365:555-557; Struhl et al., 1993, Cell 69:331).

For a general review on the Notch pathway and Notch signaling, see Artavanis-Tsakonas et al., 1995, Science 268:225-232.

Ligands, cytoplasmic effectors and nuclear elements of Notch signaling have been identified in Drosophila, and vertebrate counterparts have also been cloned (reviewed in Artavanis-Tsakonas et al., 1995, Science 268:225-232). While protein interactions between the various elements have been documented, the biochemical nature of Notch signaling remains elusive. Expression of truncated forms of Notch reveal that Notch proteins without transmembrane and extracellular domains are translocated to the nucleus both in transgenic flies and in transfected mammalian or Drosophila cells (Lieber et al., 1993, Genes and Development 7:1949-1965; Fortini et al., 1993, Nature 365:555-557; Ahmad et al., 1995, Mechanisms of Development 53:78-85; Zagouras et al., 1995, Proc. Natl. Acad. Sci. USA 92:6414-6418). Sequence comparisons between mammalian and Drosophila Notch molecules, along with deletion analysis, have found two nuclear localization sequences that reside on either side of the ankyrin repeats (Stifani et al., 1992, Nature Genetics 2:119-127; Lieber et al., 1993, Genes and Development 7:1949-1965; Kopan et al., 1994, Development 120:2385-2396). These findings prompted the speculation that Notch may be directly participating in nuclear events by means of a proteolytic cleavage and subsequent translocation of the intracellular fragment into the nucleus. However, conclusive functional evidence for such a hypothesis remains elusive (Artavanis-Tsakonas et al., 1995, Science 268:225-232).

2.2 Mam

Mastermind encodes a novel ubiquitous nuclear protein involved in the Notch pathway as shown by genetic analysis (Smoller et al., 1990, Genes Dev. 4:1688). Two human homologs of Mastermind have been cloned, MAML1 and MAML2 (Wu et al., 2000, Nature Genetics 26:484-489; see FIGS. 1-6). Mastermind contains an amino-terminal basic domain and two acid domains, one of which is in the carboxy terminus, and has been shown to localize to nuclear bodies. FIG. 5 is a schematic of the Mastermind domains and their location. Drosophila Mastermind is 1596 amino acids in length and has an unusually large number of homopolymer repeats (glutamine, glycine and asparagine) that are separated by regions of charged amino acids, an arrangement similar to nuclear regulatory proteins. Mastermind has been shown to bind to the ankyrin repeat domain of all four known mammalian Notch proteins and its expression has been shown to amplify Notch-induced transcription, and thus, Mastermind functions as a transcriptional co-activator for Notch signal transduction (Wu et al., 2000, Nature Genetics 26:484-489).

2.3 SUMO Conjugation

SUMO (small ubiquitin-related modifier) is the best characterized member of a growing family of ubiquitin-related proteins. It resembles ubiquitin in its structure, its ability to be ligated to other proteins, as well as in the mechanism of ligation. However, in contrast to ubiquitinization, often the first step on a one-way road to protein degradation, sumolation does not seem to mark proteins for degradation. In fact, sumolation may even function as an antagonist of ubiquitin in the degradation of selected proteins. The SUMO conjugation machinery is evolutionarily conserved and has been described in organisms ranging from yeast to man. SUMO first undergoes an ATP-dependent activation by a heterodimeric complex (Uba2p/Aos1p) and is conjugated to Aos1p (activating enzyme) through a thioester bond. The SUMO protein is then transferred, through another thioester bond, to a SUMO-conjugating enzyme, Ubc9. Additional components of the SUMO conjugation pathway have not been identified, and it is likely that SUMO is conjugated to a protein substrate through direct transfer from Ubc9. The types of proteins known to date that are modified by SUMO participate in a wide spectrum of nuclear processes, including nuclear transport, kinetochore and centromere function, recombination, transcription and nuclear body structure. Consequently, any protein or signaling pathway that can influence SUMO conjugation could have a profound effect on nuclear functions. For a general review of the SUMO conjugation pathway, see Melchior, 2000, Ann. Rev. Cell Dev. Biol. 16:591-526.

2.4 Mip1/Uba2p

Drosophila Uba2p (Mip1) is one of two subunits that comprise the activating enzyme for SUMO. Homologs of the Uba2p gene have been cloned from several species, including humans. See FIG. 8 for an amino acid comparison of different homologs of Uba2p.

Citation or identification of any reference in Section 2 or any other section of this application shall not be construed as an admission that such reference is available as prior art to the present invention.

### 3. SUMMARY OF THE INVENTION

The present invention is based, in part, on the discovery of interactions of Mastermind (Mam) with the Mip1, Mip30 and Mip6 proteins, as well as the isolation of Mip30 and Mip6 nucleic and amino acid sequences. The present invention is also based, in part, on the novel observation that an increase in Notch signal transduction results in an increase in sumolation in a cell, thus demonstrating the interdependence of the Notch signal transduction pathway and SUMO conjugation.

Mastermind is a member of the Notch family of proteins and is involved in the regulation of cell fate and differentiation through Notch signaling. As described in Section 2.2, supra, Mastermind binds to the ankyrin repeat domain of Notch. Mastermind also binds Mip1, Mip30 and Mip6. Mip1, also called Uba2p, which, as discussed in Sections 2.3 and 2.4, supra, is part of the SUMO conjugation machinery, in particular, one of two subunits that comprise the SUMO activating enzyme. Sumolation of cellular proteins has been shown to alter their subcellular localization and result in longer half-lives, i.e., stabilization of the proteins. Mutations resulting in aberrant sumolation, e.g., disruption of the gene encoding SUMO, leads to severe growth defects in yeast and phenotypes such as aberrant mitosis, increase in telomere length, and defects in chromosomal segregation. It is well known that the centrosome is involved in mitosis and fidelity of chromosome segregation and that malfunctioning centrosomes can lead to missegregation of the chromosomes during mitosis, which appears to be involved in tumorigenesis, i.e. cancer formation. See, e.g., Doxsey, 1998, Nat. Genet. 20:104-106. Thus, the compositions and methods of the present invention are useful in studying cell fate and differentiation and tumorigenesis, and in studying telomere regulation and chromosome segregation and for identifying modulators of cell fate and differentiation and tumorigenesis, and in identifying modulators of telomere regulation and chromosome segregation.

The present invention is directed to methods of identifying a molecule that alters Notch signal transduction in a cell comprising contacting the cell with one or more candidate molecules; and measuring the amount of sumolation in the cell, wherein an increase or decrease in the amount of sumolation relative to said amount in a cell not so contacted with one or more of the candidate molecules indicates that the candidate molecules alter Notch signal transduction. The present invention is also directed to methods of identifying a molecule that alters Notch signal transduction in a cell comprising recombinantly expressing within the cell one or more candidate molecules; and measuring the amount of sumolation in the cell, wherein an increase or decrease in the amount of sumolation relative to said amount in a cell not so contacted with one or more of the candidate molecules indicates that the candidate molecules alter Notch signal transduction. The present invention is also directed to methods of identifying a molecule that alters Notch signal transduction in a cell comprising microinjecting into the cell one or more candidate molecules; and measuring the amount of sumolation in the cell, wherein an increase or decrease in the amount of sumolation relative to said amount in a cell not so contacted with one or more of the candidate molecules indicates that the candidate molecules alter Notch signal transduction.

Sumolation, or SUMO conjugation activity, can be measured, e.g., by an increase or decrease in the conjugation of SUMO to target proteins. The total cellular complement of protein targets or specific protein targets can be analyzed. The SUMO protein can be introduced as a transgene in either an epitope-tagged form or an un-tagged form. Alternatively, the extent of endogenous SUMO conjugation activity can be assessed, e.g., using anti-SUMO antibodies, or by Western blot analysis in which the results would be amenable to quantification by densitometry. Further, since SUMO conjugation of a protein often influences the intracellular localization of the protein, an assay based upon the localization of a specific target protein can be used. Also, since SUMO conjugation of a protein often stabilizes the protein since SUMO competes with the same target lysine as ubiquitin, sumolation can be measured by measuring the stability, i.e., half-life, of the target protein, e.g., by Western blot analysis.

The present invention is directed to methods of identifying a molecule that alters sumolation activity in a cell comprising contacting the cell with one or more candidate molecules; and measuring the amount of Notch signal transduction in the cell, wherein an increase or decrease in the amount of Notch signal transduction relative to said amount in a cell not so contacted with one or more of the candidate molecules indicates that the candidate molecules alter sumolation activity. Another method of identifying a molecule that alters sumolation in a cell comprises recombinantly expressing within the cell one or more candidate molecules; and measuring the amount of Notch signal transduction in the cell, wherein an increase or decrease in the amount of Notch signal transduction relative to said amount in a cell not so contacted with one or more of the candidate molecules indicates that the candidate molecules alter sumolation activity. Yet another method of identifying a molecule that alters sumolation activity in a cell comprises microinjecting into the cell one or more candidate molecules; and measuring the amount of Notch signal transduction in the cell, wherein an increase or decrease in the amount of Notch signal transduction relative to said amount in a cell not so contacted with one or more of the candidate molecules indicates that the candidate molecules alter sumolation activity.

Notch signal transduction or Notch function can be measured using assays commonly known in the art, e.g., by the ability of Notch to activate transcription of a gene in the Enhancer of split complex, e.g., mγ, mδ, m5; or to activate transcription of vestigial, cut, or the HES1 gene. An in vitro transcription assay utilizing HES1 has been described (Wu et al., 2000, Nature Genetics 26:484-489; Jarriault et al., 1995, Nature 377:355-358). Thus, increased levels of mγ, mδ, m5, vestigial, cut or HES1 mRNA or protein indicates an increased level of Notch signal transduction or Notch function. Conversely, decreased levels of mγ, mδ, m5, vestigial, cut or HES1 mRNA or protein indicates a decreased level of Notch signal transduction or Notch function. Further, activation of Notch signal transduction results in the inhibition of differentiation of precursor cells. See, U.S. Pat. No. 5,780,300. Thus, Notch signal transduction can also be measured by assaying for differentiation of precursor cells. Maintenance of the differentiation state of the precursor cell indicates active Notch signal transduction. A change in the differentiation state of the precursor cell indicates inactive Notch signal transduction. Additionally, reporter constructs with a reporter gene under the control of a promoter containing a Notch-responsive promoter element can also be used to detect Notch signal transduction. For example, the EBNA2 response element from the TP-1 promoter can be used in such a reporter construct.

The present invention is also directed to methods of inhibiting Notch signal transduction in a cell comprising contacting the cell with an antagonist of sumolation in an amount sufficient to inhibit Notch signal transduction. Further, the present invention is directed to methods of agonizing Notch signal transduction in a cell comprising contacting the cell with an agonist of sumolation in an amount sufficient to agonize Notch signal transduction. The present invention is also directed to methods of inhibiting sumolation activity in a cell comprising contacting the cell with an antagonist of Notch signal transduction in an amount sufficient to inhibit sumolation activity, as well as, methods of agonizing sumolation activity in a cell comprising contacting the cell with an agonist of Notch signal transduction in an amount sufficient to agonize sumolation activity. Agonists and antagonists of both sumolation and Notch signal transduction are well known in the art, and can also be identified using the methods of the present invention, infra.

The present invention is directed to certain compositions comprising and methods for production of protein complexes of Mam with a protein that interacts with (i.e., binds to) Mam. As used herein, “Mam-IP” refers to a Mam-interacting protein, e.g. Mip1, Mip30, Mip6. Specifically, the invention is directed to complexes of Mam, and derivatives, fragments and analogs of Mam, with Mip1, Mip30 or Mip6, and their derivatives, fragments and analogs (a complex of Mam and Mip1 or Mam and Mip30 or Mam and Mip6 is designated as Mam:Mip1 or Mam:Mip30 or Mam:Mip6, respectively, herein). The present invention is further directed to methods of screening for proteins that interact with Mam and/or Mip1, Mip30, or Mip6, or with derivatives, fragments or analogs of Mam and/or Mip1, Mip30 or Mip6.

The present invention is also directed to Mip30 and Mip6 proteins, fragments and derivatives, and their encoding nucleic acids, as well as antibodies to the proteins, fragments and derivatives of Mip30 and Mip6.

Methods for production of the Mam:Mip1, Mam:Mip30 and Mam:Mip6 complexes, and derivatives and analogs of the complexes and/or individual proteins, e.g., by recombinant means, are also provided. Pharmaceutical compositions are also provided.

The invention is further directed to methods for modulating (i.e., inhibiting or enhancing) the activity of a Mam:Mip1, Mam:Mip30 or Mam:Mip6 complex, and/or Mip30 or Mip6. The protein components of a Mam:Mip1, Mam:Mip30 and Mam:Mip6 complexes have been implicated in physiological processes including, but not limited to, disease and disorders of cell fate and differentiation and aberrant mitotic events, such as defects in chromosome segregation. Accordingly, the present invention is directed to methods for screening Mam:Mip1, Mam:Mip30 or Mam:Mip6 complexes or Mip30 or Mip6, as well as derivatives and analogs of the complexes or Mip30 or Mip6, for the ability to alter a cell function, particularly a cell function in which Mam, Mip1, Mip30 and/or Mip6 has been implicated, as non-exclusively listed, supra.

The present invention is also directed to therapeutic and prophylactic, as well as diagnostic, prognostic, and screening methods and compositions based upon the Mam:Mip1, Mam:Mip30 or Mam:Mip6 complexes (and the nucleic acids encoding the individual proteins that participate in the complex). Therapeutic compounds of the invention include, but are not limited to, Mam:Mip1, Mam:Mip30 or Mam:Mip6 complexes, and a complex where one or both members of the complex is a derivative, fragment, homolog or analog of Mam, Mip1, Mip30 or Mip6; antibodies to and nucleic acids encoding the foregoing; and antisense nucleic acids to the nucleotide sequences encoding the complex components. Diagnostic, prognostic and screening kits are also provided.

Animal models and methods of screening for modulators (i.e., agonists, and antagonists) of the activity of Mam:Mip1, Mam:Mip30 or Mam:Mip6 complexes and/or the individual proteins are also provided.

Methods of identifying molecules that inhibit, or alternatively, that increase formation of a Mam:Mip1, Mam:Mip30 or Mam:Mip6 complex are also provided.

The methods of the present invention can be carried out either in vitro or in vivo.

### 4. BRIEF DESCRIPTION OF THE DRAWINGS

FIG. 1 sets forth the nucleotide (SEQ ID NO:1) and amino acid (SEQ ID NO:2) sequences of Drosophila Mastermind (GenBank Accession No. X54251).

FIG. 2 sets forth the nucleotide (SEQ ID NO:3) and amino acid (SEQ ID NO:4) sequences of a human homolog of Mastermind, MAML1 (GenBank Accession No. NM_{—}014757).

FIG. 3 sets forth the nucleotide (SEQ ID NO:5) and amino acid (SEQ ID NO:6) sequences of another human homolog of Mastermind, MAML2 (GenBank Accession No. AB058719).

FIG. 4 is a comparison between the amino acid sequence of Drosophila Mastermind (SEQ ID NO:2) and two human homologs of Mastermind, MAML1 (SEQ ID NO:4) and MAML2 (SEQ ID NO:6), and also sets forth a consensus sequence (SEQ ID NO:7) based on the sequence comparison.

FIG. 5 is a schematic diagram showing the basic and two acidic domains of Mastermind as well as the regions of Mastermind that are responsible for binding to Notch and to Mip1, Mip30 and Mip6, and the regions responsible for transcriptional activation and for inducing sumolation.

FIG. 6 sets forth the nucleotide (SEQ ID NO:8) and the amino acid sequences (SEQ ID NO:9) of Mip1 (Uba2p).

FIG. 7 is a comparison between amino acid sequence of Neurospora (T51083) (SEQ ID NO:10), S. pombe (T39623) (SEQ ID NO:11), S. cerevisiae (UNK_{—}68186217) (SEQ ID NO:12), human (UNK_{—}68168211) (SEQ ID NO:13), mouse (UNK_{—}681862122) (SEQ ID NO:14), Drosophila (AF193553_{—}1:) (SEQ ID NO:9), C. elegans (UNK_{—}68186214) (SEQ ID NO:15) and Arabadopsis (AC06841_{—}24:) (SEQ ID NO:16) homologs of Mip1 (Uba2p). A consensus sequence is also generated (SEQ ID NO21).

FIG. 8 is a chart setting forth the amino acid length of each Mip1 protein compared in FIG. 7, as well as the amino acid location of the UBACT repeat domain and UBA/THIF family domain for each homolog.

FIG. 9 is a schematic of the Mip1 protein showing the location of the UBA/THIF-type NAD/FAD family domain (amino acids 12-155), the UBACT repeat domain (amino acids 359-506), the bipartite nuclear localization signal (NLS) (amino acids 154-171), and Mastermind interacting domain (amino acids 458-700).

FIG. 10 sets forth the nucleotide (SEQ ID NO:17) and the amino acid sequences (SEQ ID NO:18) of Mip30.

FIG. 11 is a schematic of the Mip30 protein showing the location of the motifs present in Mip30. Three prominent motifs were identified, C2H2-type zinc fingers (amino acids 28-51, 71-97, 104-127, 341-364, 383-407, 414-437 and 482-504), an A+T hook domain (amino acids 164-176) and a bipartite nuclear localization signal (NLS) (amino acids 301-318).

FIG. 12 sets forth the nucleotide (SEQ ID NO:19) and the amino acid sequences (SEQ ID NO:20) of Mip6. The minimal Mam interacting domain of Mip6 known is amino acids 374-625.

FIG. 13 is a graph showing Mam-Mip complex driven transcription in a two-hybrid analysis in yeast. The yeast strain EGY48 was co-transformed with a plasmid encoding a Mam-Gal4 DNA binding domain fusion protein and either a plasmid encoding Mip1, Mip30 or Mip6 fused to the E. coli B42 transactivation domain, or a control plasmid (pJG4-5). The DNA binding domain was derived from plasmid pEG202. A Mam-Mip interaction is demonstrated by activation of transcription from a lacZ transgene and reported in terms of arbitrary β-galactosidase units. In the presence of glucose, the expression of Mam is repressed and only a very low level of β-galactosidase activity is detected. In the presence of galactose, the expression of Mam is induced and a large increase in β-galactosidase activity is observed with those proteins that interact with Mam. Extracts were prepared and activity measured from equal number of cells.

FIG. 14 shows that Mastermind is localized to subnuclear domains by indirect immunofluorescence analysis in 293T cells. Drosophila Mam was tagged at its amino terminus with the Flag epitope (Ciaccia and Pierce, 1992, IBI Flag Epitope 1:4-5) and expressed from the pcDNA3 vector (Invitrogen, Carlsbad, Calif.) in a human kidney epithelial cell line (293T). Mam was visualized using the anti-Flag monoclonal antibody M2 (Sigma, St. Louis, Mo.) and a Cy-2-conjugated goat anti-mouse IgG secondary antibody obtained from Jackson ImmunoResearch Laboratories, Inc., West Grove, Pa. Nuclei were counterstained with DAPI.

FIG. 15 shows that Mastermind localizes to nuclear bodies in 293T cell, as determined by co-localization with the PML oncogene product, the signature protein for nuclear bodies. Hemagglutinin (HA) epitope-tagged Mam was visualized with a rabbit polyclonal anti-HA antibody (obtained from Santa Cruz Biotechnology, Inc., Santa Cruz, Calif.) and a Cy-5-conjugated goat anti-rabbit IgG secondary antibody (obtained from Jackson ImmunoResearch Laboratories, West Grove, Pa.). PML was visualized with the PG-M3 monoclonal antibody (obtained from Santa Cruz Biotechnology, Inc., Santa Cruz, Calif.) and a Cy-2-conjugated goat anti-mouse secondary antibody (obtained from Jackson ImmunoResearch Laboratories, Inc., West Grove, Pa.).

FIG. 16 demonstrates that Mastermind induces Notch relocalization in 293T cells by indirect immunofluorescence. Mastermind and the entire intracellular domain of Drosophila Notch were co-expressed from pcDNA3 vectors (obtained from Invitrogen, Carlsbad, Calif.). Intracellular Notch was visualized with the 9C6 monoclonal antibody (Rebay, 1983, Thesis, Yale University) and a Cy-2-conjugated goat anti-mouse IgG secondary antibody. Flag epitope-tagged Mam was visualized with an anti-Flag polyclonal antibody and a Cy-5-conjugated goat anti-rabbit secondary antibody. Nuclei were counterstained with DAPI. In the absence of Mam, intracellular Notch is homogeneously distributed throughout the nucleoplasm; however, when co-expressed with Mam, intracellular Notch accumulates in nuclear bodies.

FIG. 17 shows that Mastermind induces Mip1 localization to nuclear bodies. Mip1 was tagged at its amino terminus with the HA epitope and expressed from the pcDNA3 vector. Mip1 was visualized with a rabbit polyclonal anti-HA antibody and a Cy-2-conjugated goat anti-rabbit IgG secondary antibody. Mam was visualized with the M2 anti-Flag monoclonal antibody and a Cy-2-conjugated goat anti-mouse secondary antibody. Nuclei were counterstained with DAPI. In the absence of Mam, Mip1 appears to be homogeneously distributed throughout the nucleoplasm. When co-expressed with Mam, Mip1 accumulates in nuclear bodies.

FIG. 18 is a western blot of total cell lysates prepared from transfected 293T cell showing that Mastermind is an activator of sumolation. Cells were co-transfected with an equal amount of plasmid (pcDNA3) encoding HA-tagged Drosophila SUMO protein and increasing amounts of a plasmid (pcDNA3) encoding Flag-tagged Mam. Expression of Mam increases the level of SUMO conjugation to cellular proteins. SUMO-conjugated proteins were detected with a monoclonal anti-HA antibody (obtained from BabCO, Richmod, Calif.), an HRP-conjugated goat anti-mouse IgG secondary antibody (obtained from Santa Cruz Biotechnology, Santa Cruz, Calif.) and the Super Signal DuraWest chemoluminescence detection system (Pierce, Rockford, Ill.). Blocking and antibody incubations were carried out in 1×PBS; 0.25% Tween 20; 5% non-fat dry milk (Carnation); 5% goat serum (Sigma, St. Louis, Mo., catalog # G-6767). Blots were incubated with primary and secondary antibodies for 1 hour each. Detection was performed as per manufacturer's (Pierce's) instructions.

FIGS. 19A and 19B are western blots of total cell lysates prepared from 293T cells and show that Mastermind is a general activator of SUMO conjugation activity in that Mam increases the conjugation of SUMO-1, SUMO-2 and SUMO-3 to cellular proteins. In FIG. 19A, the cells were transfected with equal amounts of a plasmid encoding HA epitope-tagged SUMO and a control plasmid (lane 1) or a plasmid encoding HA epitope-tagged SUMO and a plasmid encoding Flag epitope-tagged Mam (lane 2). In FIG. 19B, the cells were transfected with equal amounts of a plasmid encoding HA-tagged SUMO-2 and a control plasmid (lane 3), equal amounts of a plasmid encoding HA-epitope-tagged SUMO-3 and a control plasmid (lane 4), equal amounts of a plasmid encoding HA-epitope-tagged SUMO-2 and a plasmid encoding Flag epitope-tagged Mam (lane 5), and equal amounts of a plasmid encoding HA-epitope-tagged SUMO-3 and a plasmid encoding Flag epitope-tagged Mam (lane 6). Western blots were probed and developed as described for FIG. 16.

### 5. DETAILED DESCRIPTION OF THE INVENTION

The present invention is based, in part, upon the identification of proteins that interact with Mastermind, a protein involved in the Notch signal transduction pathway. The interacting proteins Mip1, Mip30 and Mip6 were found to form a complex under physiological conditions with Mam. The Mam:Mip1, Mam:Mip30 and Mam:Mip6 complexes, by virtue of the interaction, are implicated in modulating the functional activities of Mam and its binding partners, in particular, Mip1, Mip30 and Mip6. Such functional activities include physiological processes including, but not limited to, disorders of cell fate and differentiation and disorders to due aberrant chromosome segregation. The present invention is also directed to novel nucleic and amino acid sequences of Mip30 and Mip6 and methods and compositions relating thereto.

The present invention is directed to methods of identifying a molecule that alters Notch signal transduction in a cell comprising contacting the cell with one or more candidate molecules; and measuring the amount of sumolation in the cell, wherein an increase or decrease in the amount of sumolation relative to said amount in a cell not so contacted with one or more of the candidate molecules indicates that the candidate molecules alter Notch signal transduction. The present invention is also directed to methods of identifying a molecule that alters Notch signal transduction in a cell comprising recombinantly expressing within the cell one or more candidate molecules; and measuring the amount of sumolation in the cell, wherein an increase or decrease in the amount of sumolation relative to said amount in a cell not so contacted with one or more of the candidate molecules indicates that the candidate molecules alter Notch signal transduction. The present invention is also directed to methods of identifying a molecule that alters Notch signal transduction in a cell comprising microinjecting into the cell one or more candidate molecules; and measuring the amount of sumolation in the cell, wherein an increase or decrease in the amount of sumolation relative to said amount in a cell not so contacted with one or more of the candidate molecules indicates that the candidate molecules alter Notch signal transduction.

Sumolation, or SUMO conjugation activity, can be measured, e.g., by an increase or decrease in the conjugation of SUMO to target proteins. The total cellular complement of protein targets or specific protein targets can be analyzed. The SUMO protein can be introduced as a transgene in either an epitope-tagged form or an un-tagged form. Alternatively, the extent of endogenous SUMO conjugation activity can be assessed, e.g., using anti-SUMO antibodies, or by Western blot analysis in which the results would be amenable to quantification by densitometry. Further, since SUMO conjugation of a protein often influences the intracellular localization of the protein, an assay based upon the localization of a specific target protein can be used. Also, since SUMO conjugation of a protein often stabilizes the protein since SUMO competes with the same target lysine as ubiquitin, sumolation can be measured by measuring the stability, i.e., half-life, of the target protein, e.g., by Western blot analysis.

The present invention is directed to methods of identifying a molecule that alters sumolation activity in a cell comprising contacting the cell with one or more candidate molecules; and measuring the amount of Notch signal transduction in the cell, wherein an increase or decrease in the amount of Notch signal transduction relative to said amount in a cell not so contacted with one or more of the candidate molecules indicates that the candidate molecules alter sumolation activity. Another method of identifying a molecule that alters sumolation in a cell comprises recombinantly expressing within the cell one or more candidate molecules; and measuring the amount of Notch signal transduction in the cell, wherein an increase or decrease in the amount of Notch signal transduction relative to said amount in a cell not so contacted with one or more of the candidate molecules indicates that the candidate molecules alter sumolation activity. Yet another method of identifying a molecule that alters sumolation activity in a cell comprises microinjecting into the cell one or more candidate molecules; and measuring the amount of Notch signal transduction in the cell, wherein an increase or decrease in the amount of Notch signal transduction relative to said amount in a cell not so contacted with one or more of the candidate molecules indicates that the candidate molecules alter sumolation activity.

Notch signal transduction or Notch function can be measured using assays commonly known in the art, e.g., by the ability of Notch to activate transcription of a gene in the Enhancer of split complex, e.g., mγ, mδ, m5; or to activate transcription of vestigial, cut, or the HES1 gene. An in vitro transcription assay utilizing HES1 has been described (Wu et al., 2000, Nature Genetics 26:484-489; Jarriault et al., 1995, Nature 377:355-358). Thus, increased levels of mγ, mδ, m5, vestigial, cut or HES1 mRNA or protein indicates an increased level of Notch signal transduction or Notch function. Conversely, decreased levels of mγ, mδ, m5, vestigial, cut or HES1 mRNA or protein indicates a decreased level of Notch signal transduction or Notch function. Further, activation of Notch signal transduction results in the inhibition of differentiation of precursor cells. See, U.S. Pat. No. 5,780,300. Thus, Notch signal transduction can also be measured by assaying for differentiation of precursor cells. Maintenance of the differentiation state of the precursor cell indicates active Notch signal transduction. A change in the differentiation state of the precursor cell indicates inactive Notch signal transduction. See U.S. Pat. Nos. 5,780,300 and 6,083,904 for methods of measuring the differentiation state of a cell and changes thereof based on Notch signal transduction. Additionally, reporter constructs with a reporter gene under the control of a promoter containing a Notch-responsive promoter element can also be used to detect Notch signal transduction. For example, the EBNA2 response element from the TP-1 promoter can be used in such a reporter construct.

The present invention is also directed to methods of inhibiting Notch signal transduction in a cell comprising contacting the cell with an antagonist of sumolation in an amount sufficient to inhibit Notch signal transduction. Further, the present invention is directed to methods of agonizing Notch signal transduction in a cell comprising contacting the cell with an agonist of sumolation in an amount sufficient to agonize Notch signal transduction. The present invention is also directed to methods of inhibiting sumolation activity in a cell comprising contacting the cell with an antagonist of Notch signal transduction in an amount sufficient to inhibit sumolation activity, as well as, methods of agonizing sumolation activity in a cell comprising contacting the cell with an agonist of Notch signal transduction in an amount sufficient to agonize sumolation activity. Agonists and antagonists of both sumolation and Notch signal transduction are well known in the art, and can also be identified using the methods of the present invention, infra. For example, an antagonist of sumolation is a dominant negative form a Mip1, or other protein in the sumolation conjugation pathway. An illustrative example of a dominant negative form of Mip1 is a form that contains a mutated ADP binding domain such that ADP does not bind. Agonists of Notch include, but are not limited to dominant active forms of Notch, including the intracellular domain of Notch, Delta and Serrate. An illustrative dominant negative form of Notch is a form which lacks the intracellular domain. See International Publications WO 00/02897, WO 97/01571, WO96/27610 and WO 97/18822 for illustrative examples of Notch signal transduction pathway agonists and antagonists. Other antagonists of both Notch signal transduction and SUMO conjugation are antibodies which are specific for the members of the pathway, e.g., anti-Notch, anti-Mip 1, anti-Ubc9. Other antagonists include antisense nucleic acids which bind to and block translation of mRNAs encoding members of the pathway.

The present invention is directed to methods of screening for proteins that interact with (e.g., bind to) Mastermind (Mam). The invention further relates to Mam complexes, in particular Mam complexes with one of the following proteins: Mip1, Mip30 or Mip6. The invention further relates to complexes of derivatives, analogs and fragments of Mam, with Mip1, Mip30 or Mip6 or derivatives, analogs and fragments thereof of these Mam interacting proteins (“Mam-IPs”). In a preferred embodiment such complexes bind an anti-Mam:Mam-IP complex antibody. In a specific embodiment, complexes of human Mam with a human Mam-IP protein are provided.

The invention also provides methods of producing and/or isolating Mam:Mam-IP complexes. In a specific embodiment, the invention provides methods of using recombinant DNA techniques to express Mam and its binding partner (or fragments, derivatives or homologs of one or both members of the complex) either where both binding partners are under the control of one heterologous promoter (i.e., a promoter not naturally associated with the native gene encoding the particular complex component) or where each is under the control of a separate heterologous promoter.

The present invention also provides the nucleotide sequence of Mip30 and Mip-6, and their encoded amino acid sequences. The invention further relates to a Mip30 or Mip6 protein, derivatives (including but not limited to fragments) and homologs and analogs thereof, as well as to nucleic acids encoding the Mip30 or Mip6 protein, derivatives, fragments and homologs. The invention further provides for a Mip30 or Mip6 protein and gene encoding the protein, from many different species, particularly vertebrates, and more particularly mammals. In a preferred embodiment, the Mip30 or Mip6 protein and gene is of human origin. Production of the foregoing proteins and derivatives, e.g., by recombinant methods, is also provided in the present invention.

The present invention further relates to a Mip30 or Mip6 derivative or analog that is functionally active, i.e., capable of displaying one or more known functional activities associated with a full length (wild-type) Mip30 or Mip6. Such functional activities include, but are not limited to, the ability to form a complex with Mam, antigenicity [ability to bind (or compete with Mip30 or Mip6 for binding) to an anti-Mip30 or anti-Mip6 antibody, respectively], immunogenicity (ability to generate an antibody that binds to Mip30 or Mip6, respectively), etc.

Methods of diagnosis, prognosis, and screening for diseases and disorders associated with aberrant levels of a Mam:Mam-IP complex, or aberrant levels of a Mip30 or Mip6 protein, are provided. The invention also provides methods of treating or preventing diseases or disorders associated with aberrant levels of a Mam:Mam-IP complex or with aberrant levels of a Mip30 or Mip6 protein, or aberrant levels of activity of one or more of the components of the complex, comprising administration of the Mam:Mam-IP complex, or administration of the Mip30 or Mip6 protein, or administration of modulators of Mam:Mam-IP complex formation or activity (e.g., antibodies that bind the Mam:Mam-IP complex, or non-complexed Mam or its binding partner or a fragment thereof—preferably the fragment containing the portion of Mam or the Mam-IP that is directly involved in complex formation) The methods also include administering mutants of Mam or the Mam-IP that increase or decrease binding affinity, administering small molecule inhibitors/enhancers of complex formation, or administering antibodies that either stabilize or neutralize the complex, etc.

Methods of assaying a Mam:Mam-IP complex, or of assaying a Mip30 or Mip6 protein, for activity as therapeutics or diagnostics as well as methods of screening for Mam:Mam-IP complex, Mip30 or Mip6 modulators (i.e., inhibitors, agonists and antagonists) are also provided.

The methods of the present invention can be performed either in vitro or in vivo.

For clarity of disclosure, and not by way of limitation, the detailed description of the invention is divided into the subsections which follow.

5.1 Mam:Mam-IP Complexes and Mip30 and Mip6 Proteins, Derivatives and Analogs

The present invention provides Mam:Mam-IP complexes, and in particular aspects, complexes of Mam and Mip1, Mam and Mip30 and Mam:Mip6. In a preferred embodiment, the Mam:Mam-IP complex is a complex of human proteins.

The invention also relates to complexes of derivatives (including fragments) and analogs of Mam with a Mam-IP, complexes of Mam with derivatives (including fragments) and analogs of a Mam-IP, and complexes of derivatives (including fragments) and analogs of Mam and derivatives (including fragments) and analogs of a Mam-IP. As used herein, fragment, derivative or analog of a Mam:Mam-IP complex includes a complex wherein one or both members of the complex is a fragment(s), derivative(s) or analog(s) of the wild-type Mam or Mam-IP protein. Preferably, the Mam:Mam-IP complex in which one or both members of the complex is a fragment, derivative or analog of the wild type protein is a functionally active Mam:Mam-IP complex. In particular aspects, the native proteins, derivatives or analogs of Mam and/or the Mam-IP are from animals, e.g., mouse, rat, pig, cow, dog, monkey, human, fly, frog. In another aspect the native proteins, derivatives or analogs of Mam and/or the Mam-IP are from plants.

Accordingly, the present invention provides methods of screening Mam:Mam-IP complexes, particularly complexes of Mam with Mip1, Mip30 and Mip6 proteins, as well as derivatives and analogs of the Mam:Mam-IP complexes, and methods of screening Mip30 and Mip6 proteins for the ability to alter cell functions, particularly those cell functions in which Mam and/or a Mam-IP has been implicated. Such functions include, but not limited to, physiological processes such as signal transduction, post-translational protein modification, and pathological processes such as degenerative disorders including neurodegenerative disease, hyperproliferative disorders including tumorigenesis and tumor progression.

Other functions of the complexes, aside from the ability to alter cellular function, include binding to an anti-Mam:Mam-IP complex antibody, as well as other activities as described in the art. For example, derivatives or analogs of the Mam:Mam-IP complex that have the desired immunogenicity or antigenicity can be used in immunoassays, for immunization, for inhibition of Mam:Mam-IP complex activity, etc. Derivatives or analogs of the Mam:Mam-IP complex that retain or enhance, or alternatively lack or inhibit, a property of interest, e.g., participation in a Mam:Mam-IP complex, can be used as inducers, or inhibitors, respectively, of such a property and its physiological correlates. A specific embodiment relates to a Mam:Mam-IP complex of a fragment of a Mam protein and/or a fragment of a Mam-IP protein that can be bound by an anti-Mam and/or anti-Mam-IP antibody or by an antibody specific for a Mam:Mam-IP complex, when such fragment is included in a Mam:Mam-IP complex.

Fragments and other derivatives or analogs of Mam:Mam-IP complexes can be tested for the desired activity by procedures known in the art, including but not limited to the assays described in Section 5.7, infra.

The invention further relates to Mip30 or Mip6 protein as well as derivatives and homologs and analogs of Mip30 or Mip6 protein. In one embodiment a human Mip30 or Mip6 gene and protein is provided. In specific aspects, the native protein, fragment, derivative or analog of Mip30 or Mip6 protein is from animals, e.g., mouse, rat, pig, cow, dog, monkey, human, fly, or frog. In another aspect, the native protein, fragment, derivative or analog of Mip30 or Mip6 protein is from plants. In other specific embodiments, the fragment, derivative or analog is functionally active, i.e., capable of exhibiting one or more functional activity associated with wild type Mip30 or Mip6 protein, e.g., ability to bind Mam, immunogenicity or antigenicity.

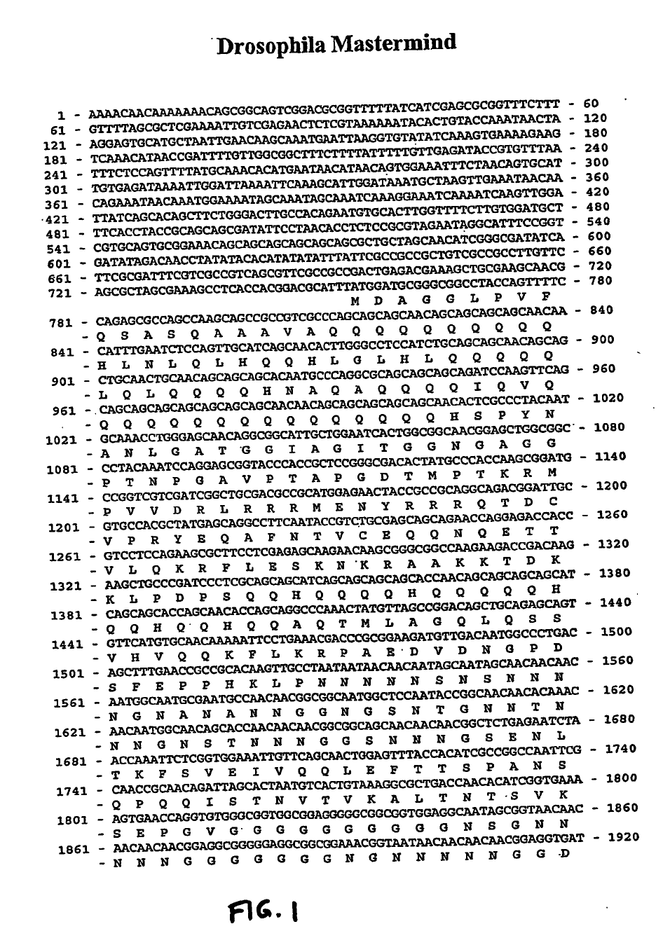

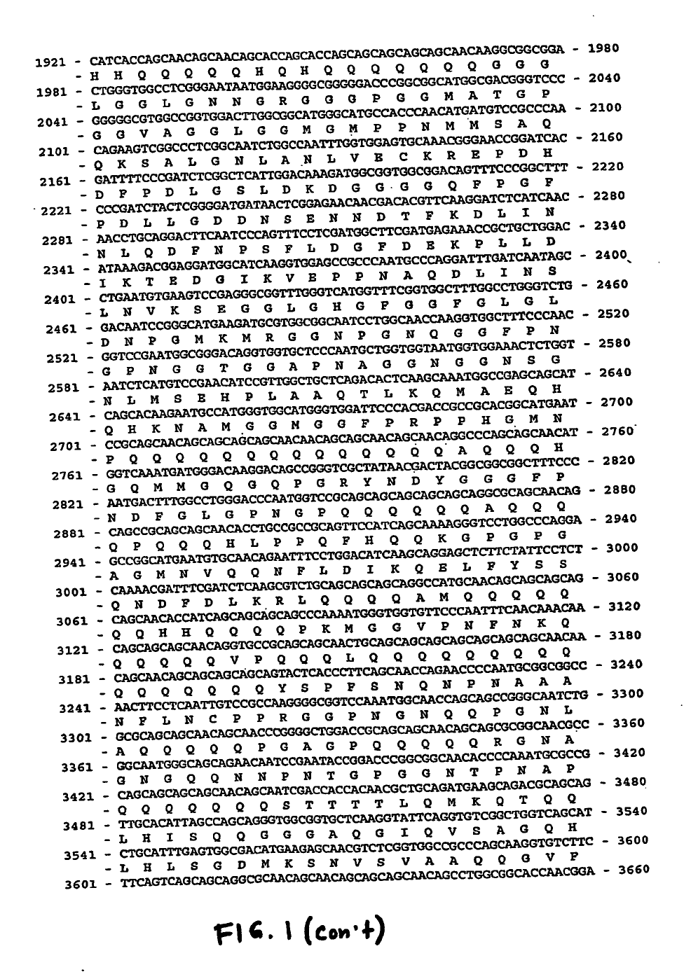

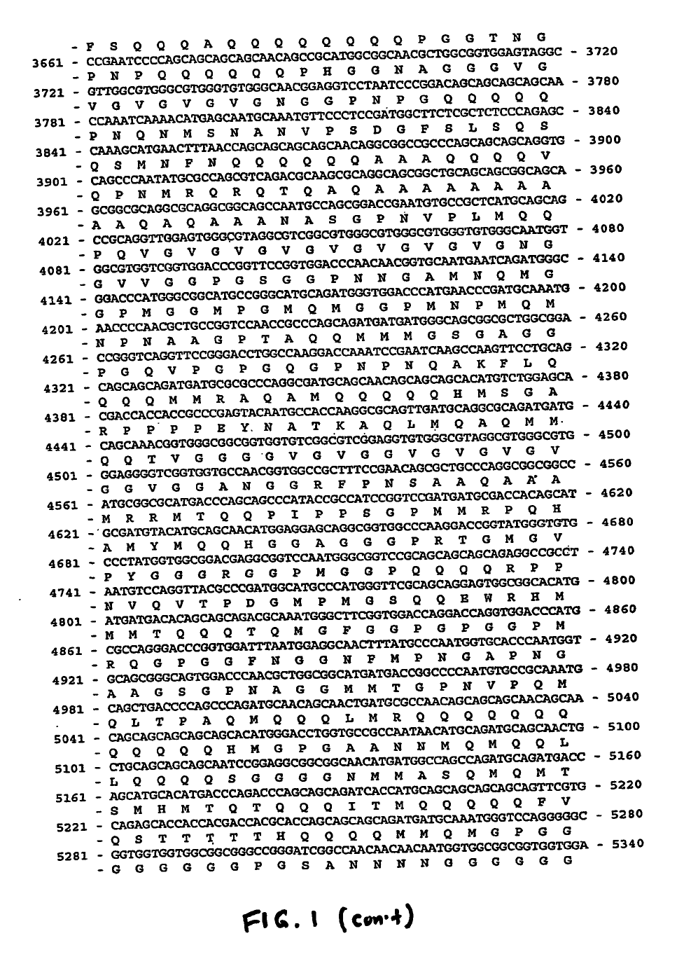

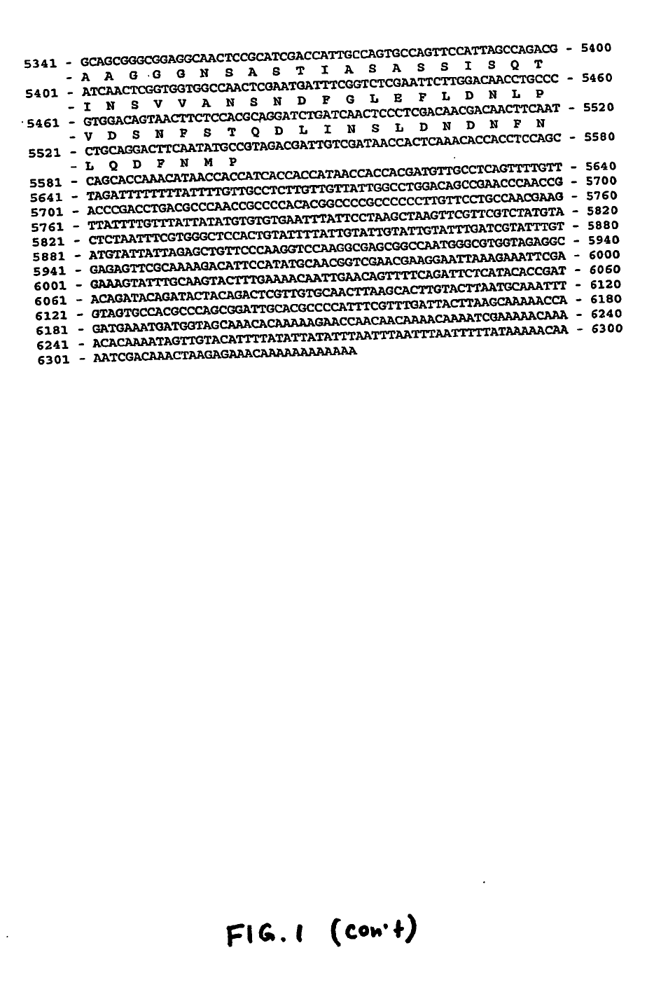

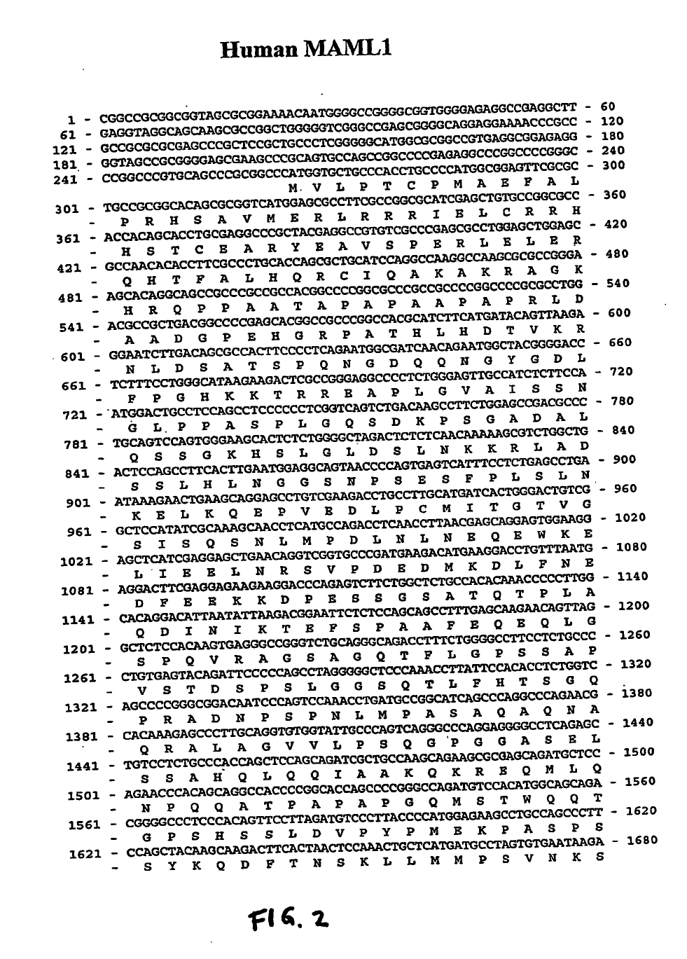

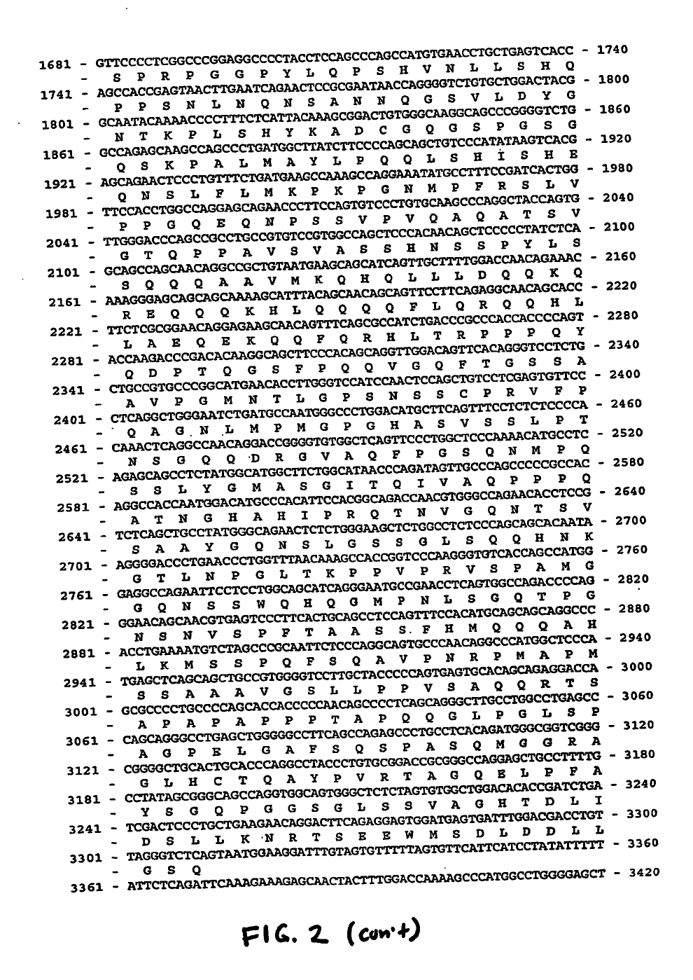

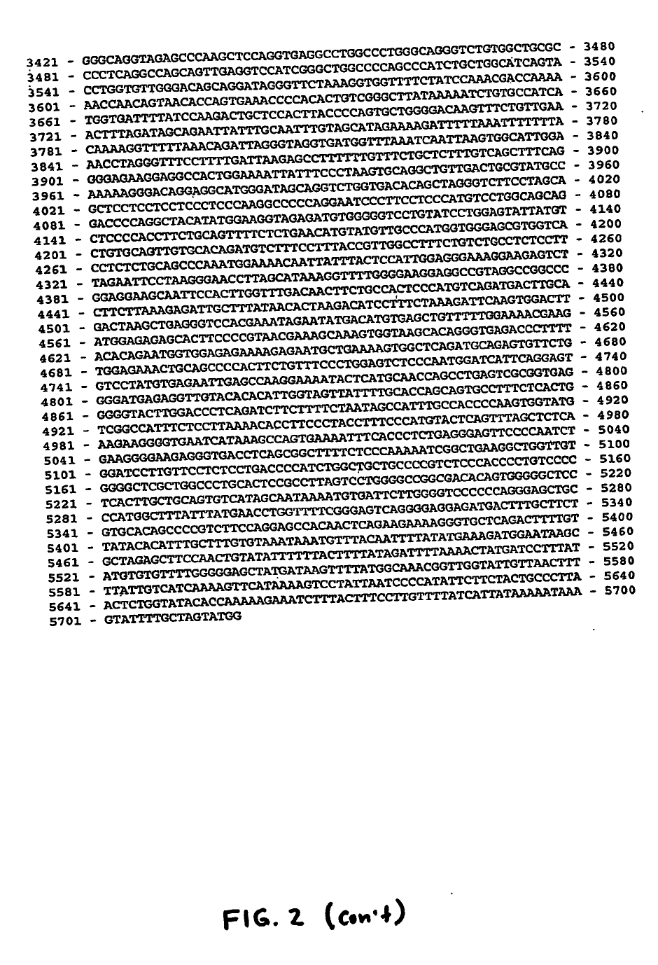

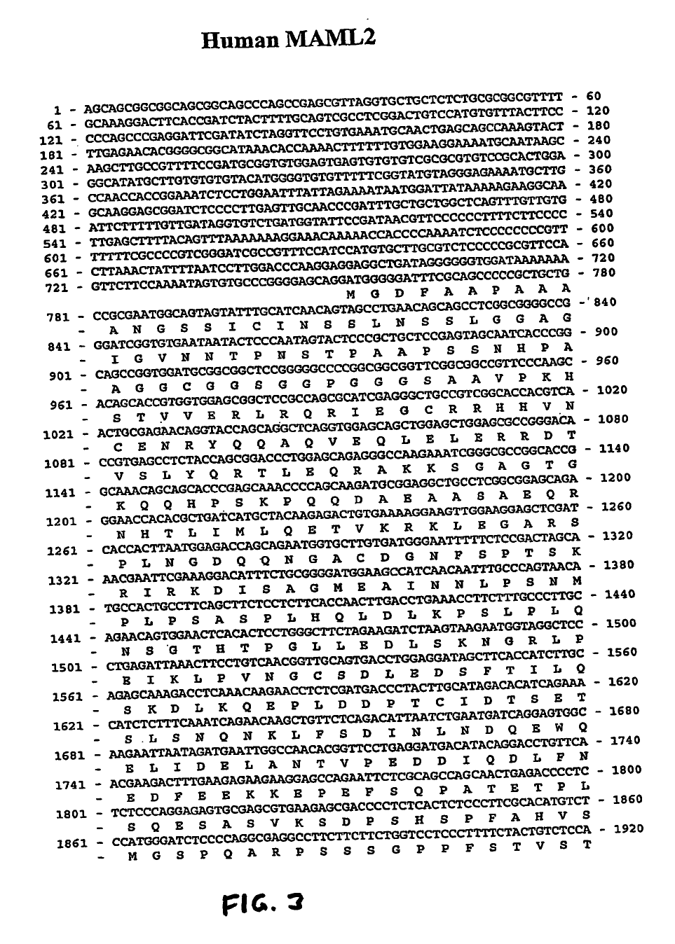

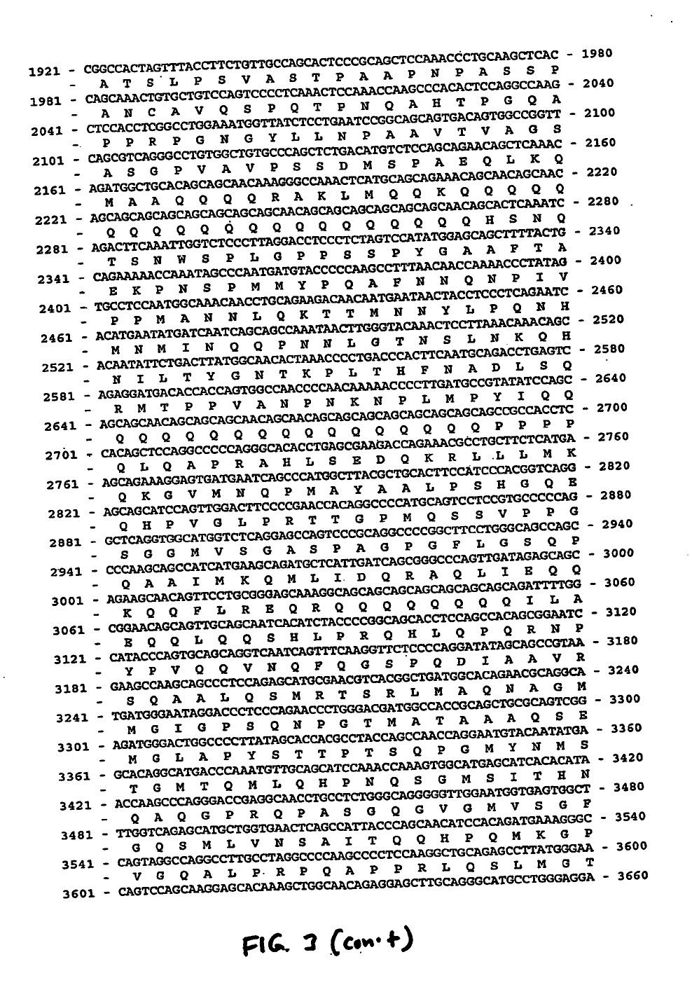

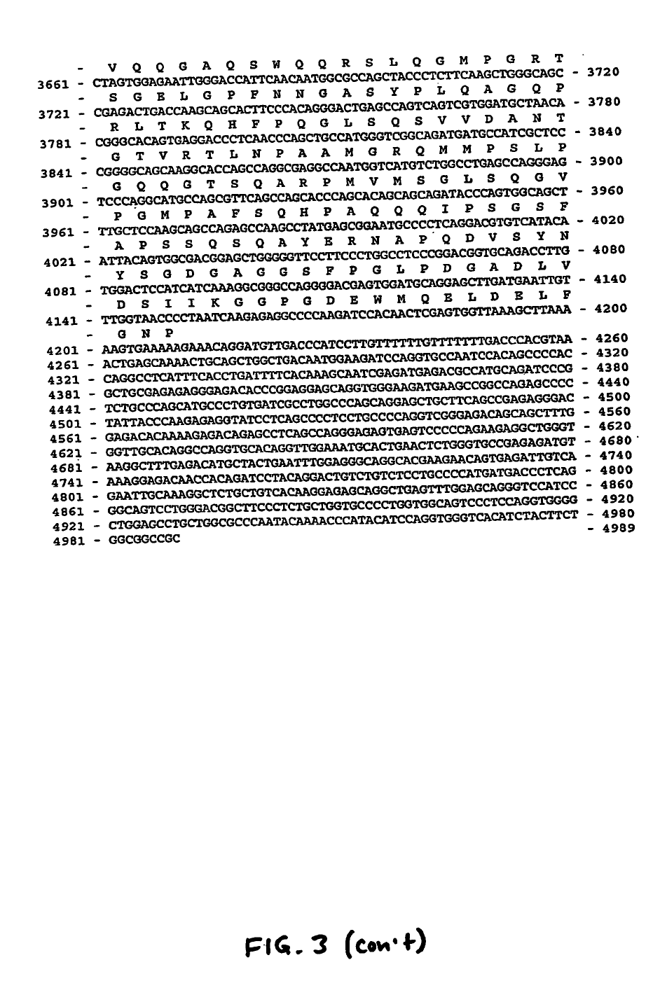

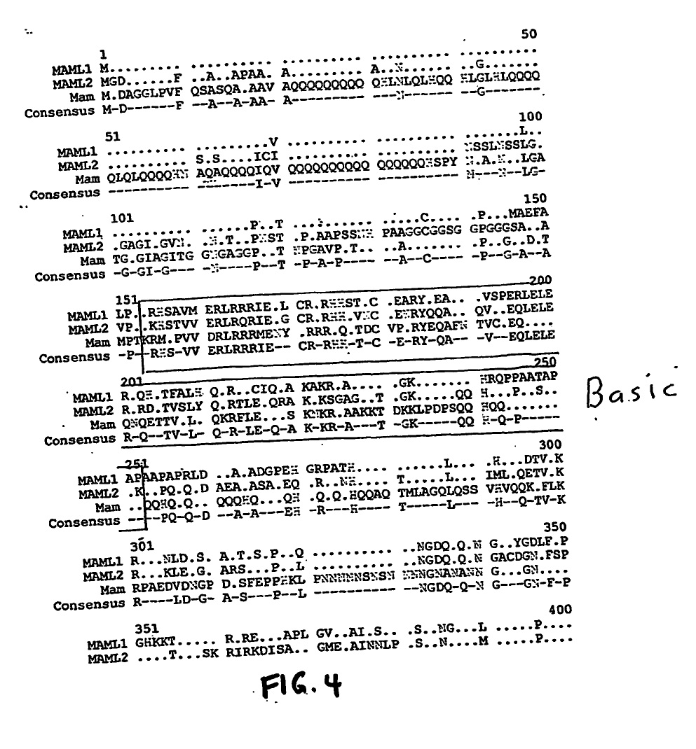

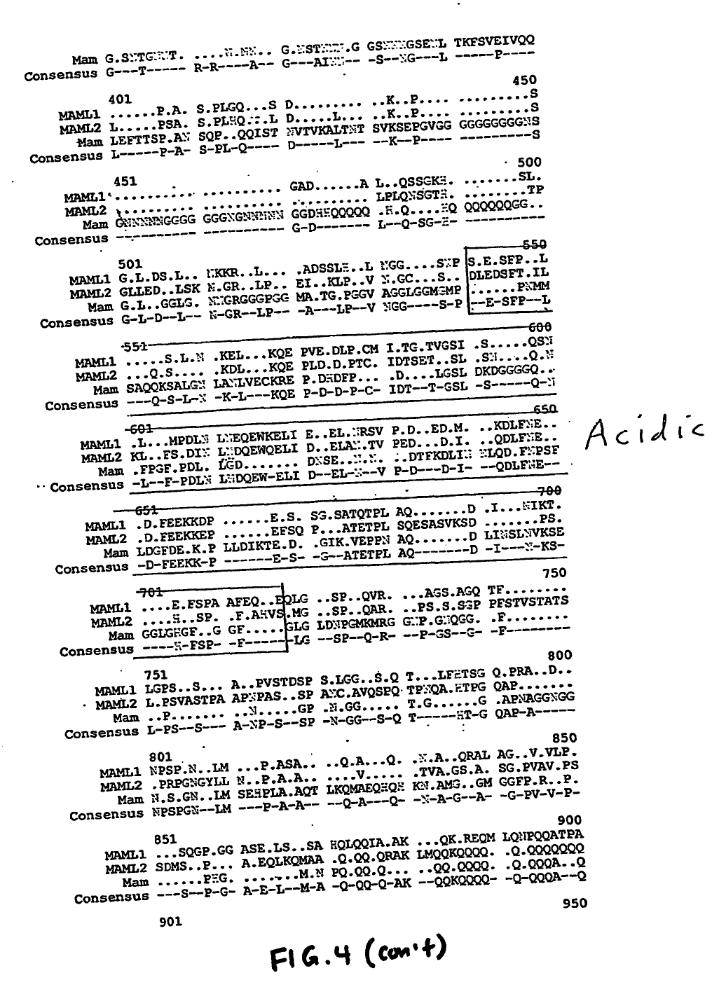

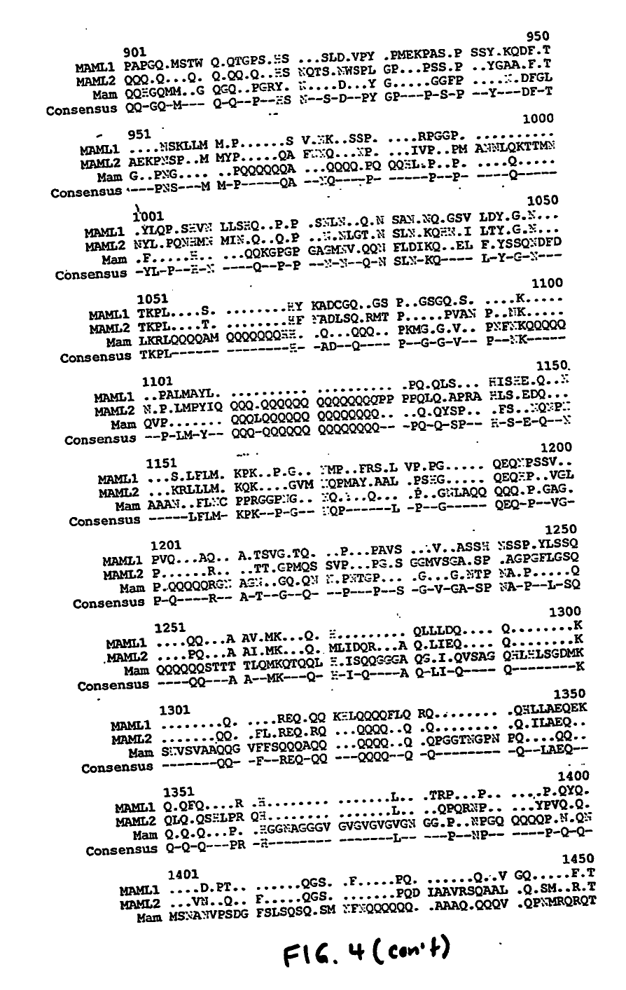

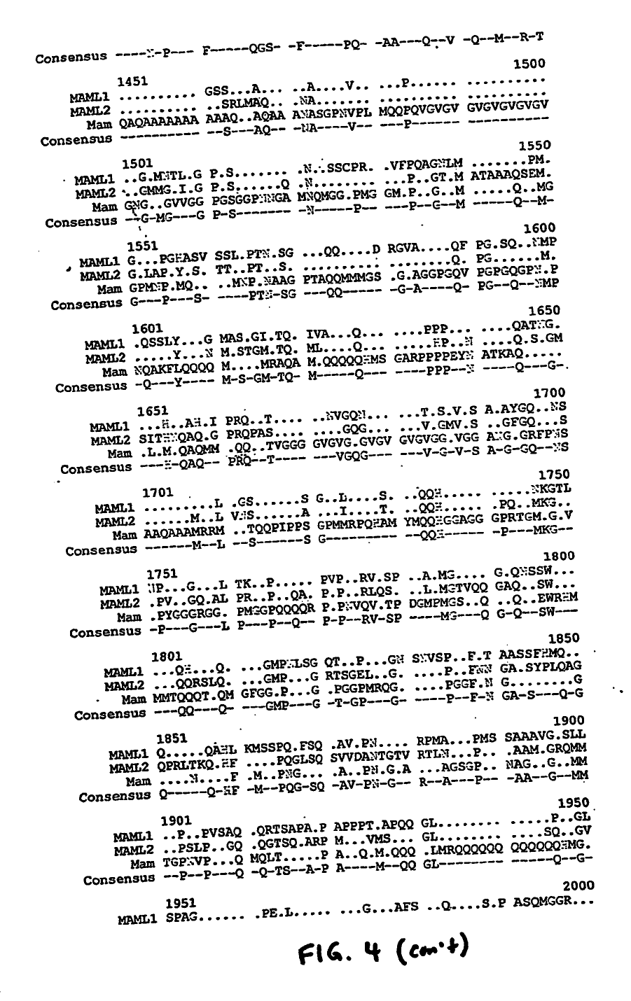

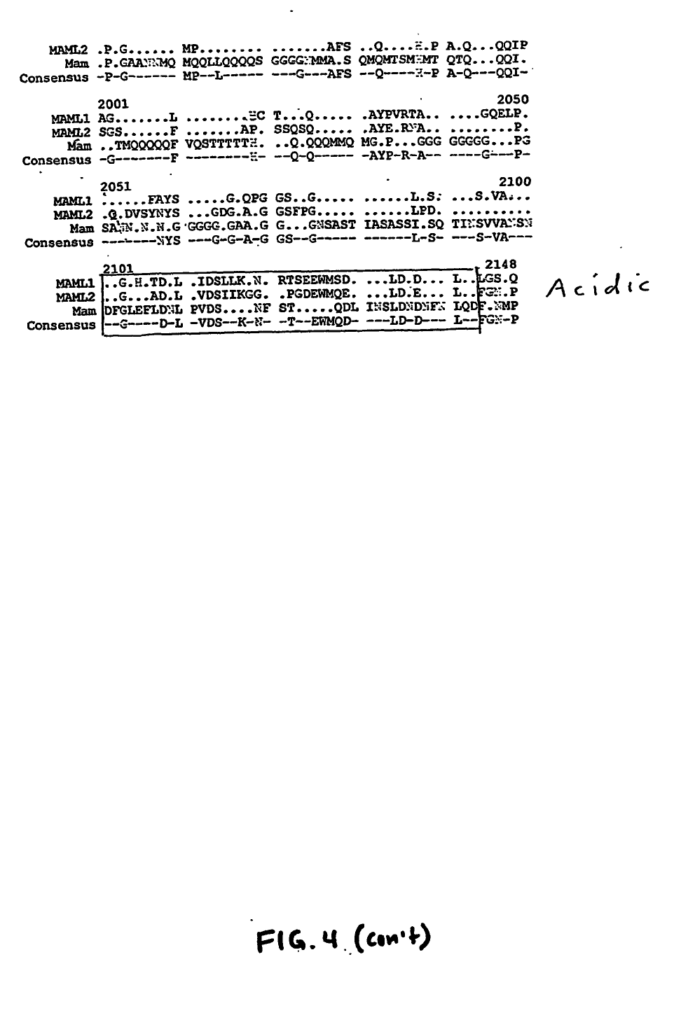

The nucleotide sequences encoding Mam and Mip1 from several species, including humans, are known and are provided in FIGS. 1-4 and 6-7, respectively. Nucleic acids encoding Mam, Mip1, Mip30 or Mip6 can be obtained by any method known in the art, e.g, by PCR amplification using synthetic primers hybridizable to the 3′ and 5′ ends of the sequence and/or by cloning from a cDNA or genomic library using an oligonucleotide specific for the gene sequence, e.g., as described in Section 5.2, infra. Due to the degeneracy of the genetic code, the term “Mam, Mip1, Mip30 or Mip6 gene”, as used herein, refers not only to the naturally occurring nucleotide sequence but also encompasses all the other degenerate DNA sequences that encode a Mam, Mip1, Mip30 or Mip6 polypeptide, respectively. Computer programs, such as Entrez, can be used to browse the database, and retrieve any amino acid sequence and genetic sequence data of interest by accession number. These databases can also be searched to identify sequences with various degrees of similarities to a query sequence using programs, such as FASTA and BLAST, which rank the similar sequences by alignment scores and statistics. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Altschul et al., 1997, supra). When utilizing BLAST, Gapped BLAST, and PSI-Blast programs, the default parameters of the respective programs (e.g., XBLAST and NBLAST) can be used (see http://www.ncbi.nlm.nih.gov).

Homologs, e.g., of nucleic acids encoding Mam, Mip1, Mip30 or Mip6 of species other than human, or other related sequences, e.g., paralogs, can be obtained by low, moderate or high stringency hybridization with all or a portion of the particular sequence as a probe using methods well known in the art for nucleic acid hybridization and cloning, e.g., as described in Section 5.2, infra, for Mip30, or Mip6 nucleotide sequences.

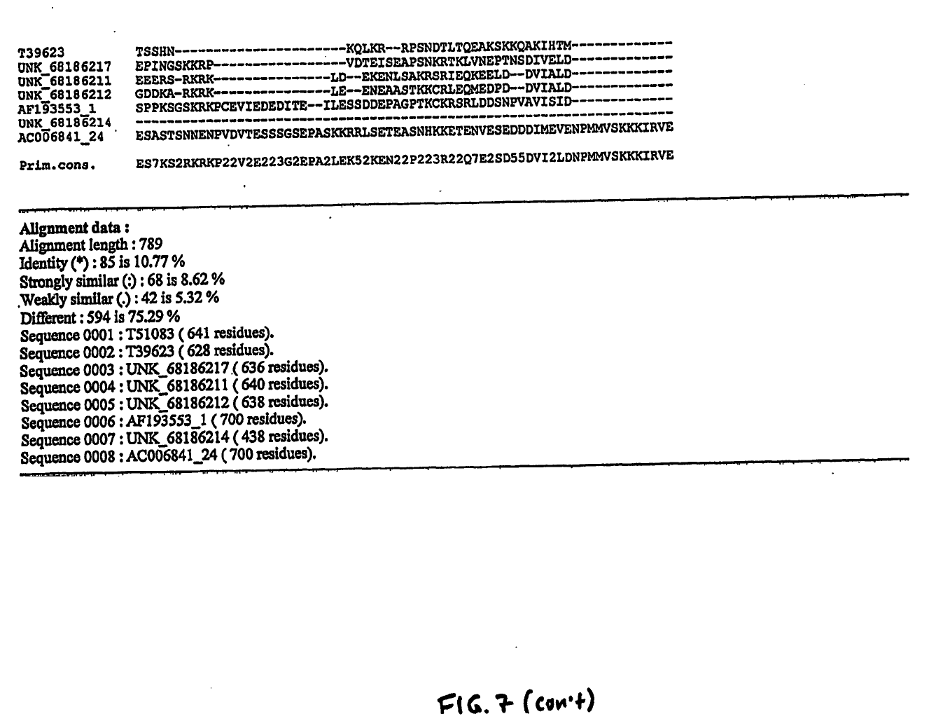

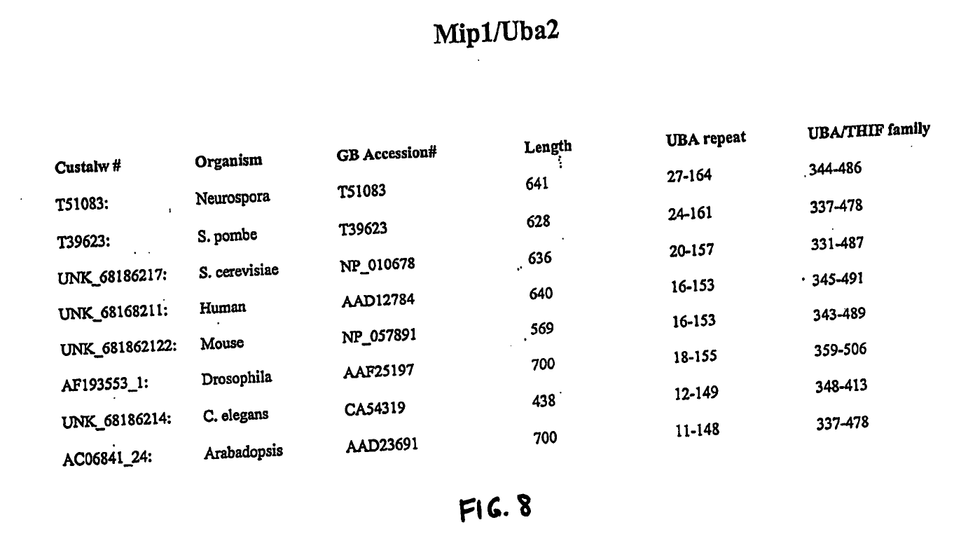

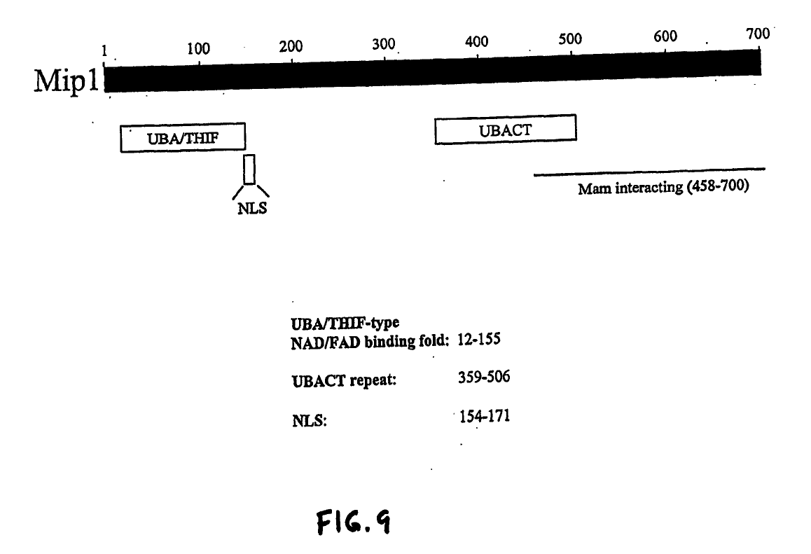

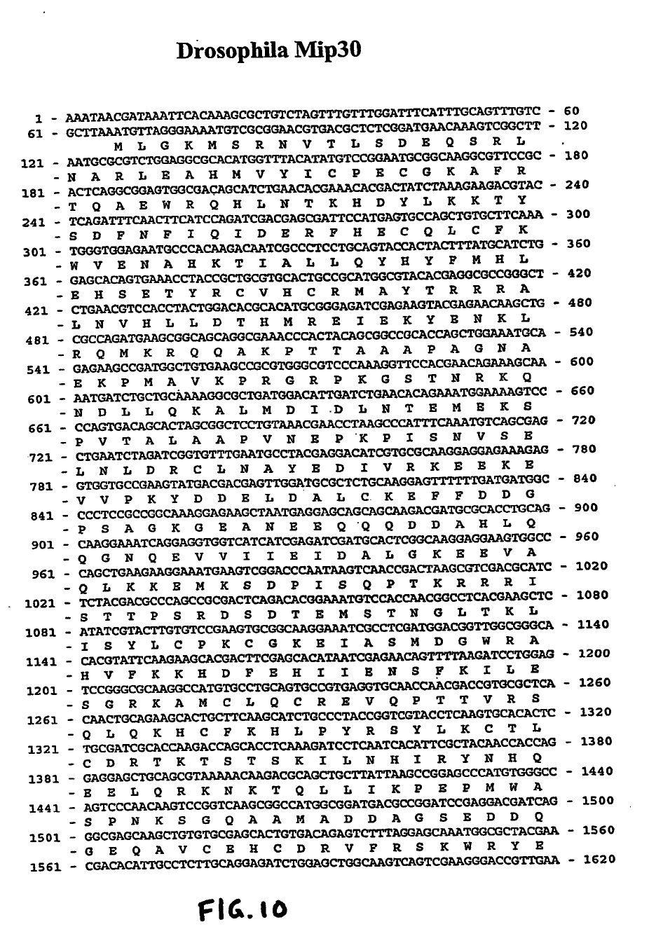

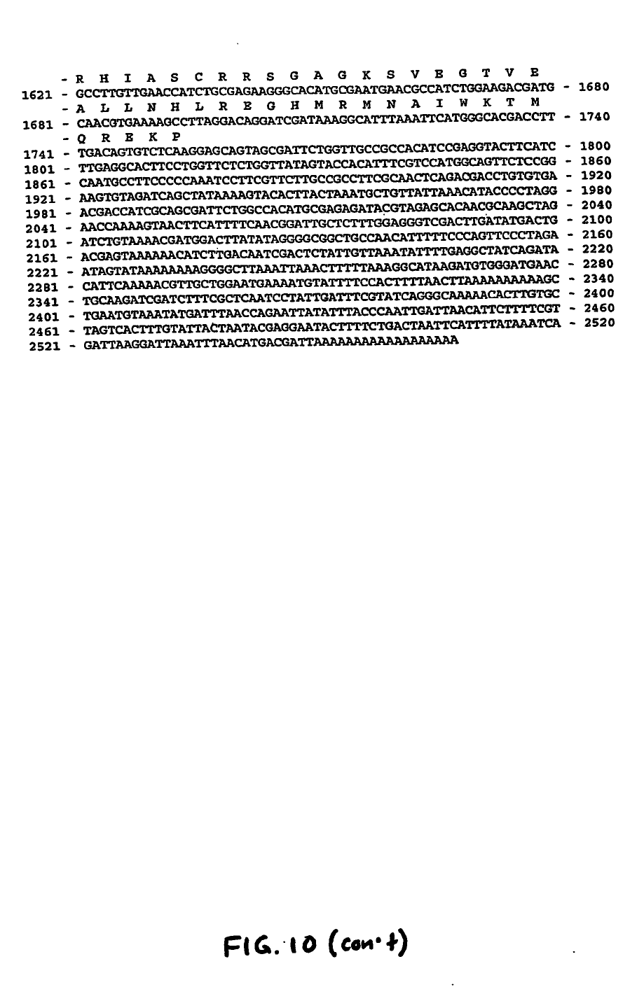

The Mam, Mip1, Mip30 or Mip6 proteins as depicted in FIGS. 1-12, (SEQ ID NOS:2, 4, 6, 7 (Mam); SEQ ID NOS:9, 10, 11, 12, 13, 14, 15, 16 (Mip1); SEQ ID NO:18 (Mip30); and SEQ ID NO:20 (Mip6)) either alone or in a complex, can be obtained by methods well known in the art for protein purification and recombinant protein expression. For recombinant expression of one or more of the proteins, the nucleic acid containing all or a portion of the nucleotide sequence encoding the protein can be inserted into an appropriate expression vector, i.e., a vector that contains the necessary elements for the transcription and translation of the inserted protein coding sequence. The necessary transcriptional and translational signals can also be supplied by the native promoter for Mam or any Mam-IP genes, and/or their flanking regions.

A variety of host-vector systems may be utilized to express the protein coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors or bacteria transformed with bacteriophage DNA, plasmid DNA, or cosmid DNA. The expression elements of vectors vary in their strengths and specificities. Depending on the host-vector system utilized, any one of a number of suitable transcription and translation elements may be used.

In a preferred embodiment, a Mam:Mam-IP complex is obtained by expressing the entire Mam sequence and a Mam-IP coding sequence in the same cell, either under the control of the same promoter or under two separate promoters. In yet another embodiment, a derivative, fragment or homolog of Mam and/or a derivative, fragment or homolog of a Mam-IP are recombinantly expressed. Preferably the derivative, fragment or homolog of Mam and/or of the Mam-IP protein forms a complex with a binding partner identified by a binding assay, such as the modified yeast two hybrid system described in Section 5.8.1 infra, and more preferably forms a complex that binds to an anti-Mam:Mam-IP complex antibody.

Any of the methods described in Section 5.2, infra, for the insertion of DNA fragments into a vector may be used to construct expression vectors containing a chimeric gene consisting of appropriate transcriptional/translational control signals and protein coding sequences. These methods may include in vitro recombinant DNA and synthetic techniques and in vivo recombinants (genetic recombination). Expression of nucleotide sequences encoding Mam and a Mam-IP (e.g., Mip1, Mip30, Mip6, or a derivative, fragment or homolog thereof), may be regulated by a second nucleotide sequence so that the gene or gene fragment thereof is expressed in a host transformed with the recombinant DNA molecule(s). For example, expression of the proteins may be controlled by any promoter/enhancer known in the art. In a specific embodiment, the promoter is not native to the gene for Mam or for Mam-IP.

Promoters which may be used include but are not limited to the SV40 early promoter (Bernoist and Chambon, 1981, Nature 290:304-310); the promoter contained in the 3′ long terminal repeat of Rous sarcoma virus (Yamamoto et al., 1980, Cell 22:787-797); the Herpes thymidine kinase promoter (Wagner et al., 1981, Proc. Natl. Acad. Sci. USA 78:1441-1445); the regulatory sequences of the metallothionein gene (Brinster et al., 1982, Nature 296:39-42); prokaryotic expression vectors such as the β-lactamase promoter (Villa-Kamaroff et al., 1978, Proc. Natl. Acad. Sci. USA 75:3727-3731) or the tac promoter (DeBoer et al., 1983, Proc. Natl. Acad. Sci. USA 80:21-25, see also Useful Proteins from Recombinant Bacteria: in Scientific American 1980, 242:79-94); plant expression vectors comprising the nopaline synthetase promoter (Herrar-Estrella et al., 1984, Nature 303:209-213) or the cauliflower mosaic virus 35S RNA promoter (Garder et al., 1981, Nucleic Acids Res. 9:2871), and the promoter of the photosynthetic enzyme ribulose bisphosphate carboxylase (Herrera-Estrella et al., 1984, Nature 310:115-120); promoter elements from yeast and other fungi such as the Gal4 promoter, the alcohol dehydrogenase promoter, the phosphoglycerol kinase promoter, the alkaline phosphatase promoter; and the following animal transcriptional control regions that exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald 1987, Hepatology 7:425-515), insulin gene control region which is active in pancreatic beta cells (Hanahan et al., 1985, Nature 315:115-122), immunoglobulin gene control region which is active in lymphoid cells (Grosschedl et al., 1984, Cell 38:647-658; Adams et al., 1985, Nature 318:533-538; Alexander et al., 1987, Mol. Cell Biol. 7:1436-1444), mouse mammary tumor virus control region which is active in testicular, breast, lymphoid and mast cells (Leder et al., 1986, Cell 45:485-495), albumin gene control region which is active in liver (Pinckert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58), alpha-1 antitrypsin gene control region which is active in liver (Kelsey et al., 1987, Genes and Devel. 1: 161-171), beta globin gene control region which is active in myeloid cells (Mogram et al., 1985, Nature 315:338-340; Kollias et al., 1986, Cell 46:89-94), myelin basic protein gene control region which is active in oligodendrocyte cells of the brain (Readhead et al., 1987, Cell 48:703-712), myosin light chain-2 gene control region which is active in skeletal muscle (Sani 1985, Nature 314:283-286), and gonadotrophic releasing hormone gene control region which is active in gonadotrophs of the hypothalamus (Mason et al., 1986, Science 234:1372-1378).

In a specific embodiment, a vector is used that comprises a promoter operably linked to nucleotide sequences encoding Mam and/or a Mam-IP (e.g., Mip1, Mip30, Mip6), or a fragment, derivative or homolog thereof, one or more origins of replication, and optionally, one or more selectable markers (e.g., an antibiotic resistance gene). In a preferred embodiment, a vector is used that comprises a promoter operably linked to nucleotide sequences encoding both Mam and a Mam-IP, one or more origins of replication, and optionally, one or more selectable markers.

In another specific embodiment, an expression vector containing the coding sequence, or a portion thereof, of Mam and a Mam-IP either together or separately, is made by subcloning the gene sequences into the EcoRI restriction site of one of the three pGEX vectors (glutathione S-transferase expression vectors; Smith and Johnson, 1988, Gene 7:3140; Promega Corp., Madison, Wis.). This allows for the expression of products in the correct reading frame.

Expression vectors containing the sequences of interest can be identified by three general approaches: (a) nucleic acid hybridization, (b) presence or absence of marker gene function, and (c) expression of the inserted sequences. In the first approach, Mam, Mip1, Mip30 or Mip6, or other Mam-IP sequences can be detected by nucleic acid hybridization to probes comprising sequences homologous and complementary to the inserted sequences. In the second approach, the recombinant vector/host system can be identified and selected based upon the presence or absence of certain marker functions (e.g., binding to an anti-Mam, anti-Mam-IP, or anti-Mam:Mam-IP complex antibody, resistance to antibiotics, occlusion body formation in baculovirus, etc.) caused by insertion of the sequences of interest in the vector. For example, if a Mam or Mam-IP gene, or portion thereof, is inserted within the marker gene sequence of the vector, recombinants containing the Mam or Mam-IP fragment will be identified by the absence of the marker gene function. In the third approach, recombinant expression vectors can be identified by assaying for Mam, Mip1, Mip30 or Mip6 products expressed by the recombinant vector. Such assays can be based, for example, on the physical or functional properties of the interacting species in in vitro assay systems, e.g., formation of a Mam:Mam-IP complex or immunoreactivity to antibodies specific for the protein.

Once recombinant Mam, Mip1, Mip30 or Mip6 molecules are identified and the complexes or individual proteins are isolated, several methods known in the art can be used to propagate them. Once a suitable host system and growth conditions have been established, recombinant expression vectors can be propagated and amplified in quantity. As previously described, the expression vectors or derivatives which can be used include, but are not limited to, human or animal viruses such as vaccinia virus or adenovirus; insect viruses such as baculovirus; yeast vectors; bacteriophage vectors such as lambda phage; and plasmid and cosmid vectors.

In addition, a host cell strain may be chosen that modulates the expression of the inserted sequence, or modifies or processes the expressed protein in the specific fashion desired. Expression from certain promoters can be elevated in the presence of certain inducers; thus, expression of the genetically-engineered Mam and/or Mam-IP may be controlled. Furthermore, different host cells have characteristic and specific mechanisms for the translational and post-translational processing and modification (e.g., glycosylation, phosphorylation, etc.) of proteins. Appropriate cell lines or host systems can be chosen to ensure the desired modification and processing of the foreign protein is achieved. For example, expression in a bacterial system can be used to produce an unglycosylated core protein, while expression in mammalian cells can ensure native glycosylation of a heterologous mammalian protein. Furthermore, different vector/host expression systems may effect processing reactions to different extents.

In other specific embodiments, the Mam and/or Mam-IP, or fragment, homolog or derivative thereof, may be expressed as a fusion or chimeric protein product comprising the protein, fragment, homolog, or derivative joined via a peptide bond to a heterologous protein sequence of a different protein. Such chimeric products can be made by ligating the appropriate nucleic acids encoding the desired amino acids to each other in the proper coding frame by methods known in the art, and expressing the chimeric products in a suitable host by methods commonly known in the art. Alternatively, such a chimeric product can be made by protein synthetic techniques, e.g., by use of a peptide synthesizer. Chimeric genes comprising portions of Mam and/or Mip1, Mip30, or Mip6, fused to any heterologous protein-encoding sequences may be constructed. A specific embodiment relates to a chimeric protein comprising a fragment of Mam and/or a Mam-IP, or a fragment of Mip1, Mip30, or Mip6 protein, of at least six amino acids.

In a specific embodiment, fusion proteins are provided that contain the interacting domains of the Mam protein and a Mam-IP (e.g., Mip1, Mip30 and Mip6) and/or, optionally, a hetero-functional reagent, such as a peptide linker between the two domains, where such a reagent promotes the interaction of Mam and Mam-IP binding domains. These fusion proteins may be particularly useful where the stability of the interaction is desirable (due to the formation of the complex as an intra-molecular reaction), for example in production of antibodies specific to the Mam:Mam-IP complex.

In particular, Mam and/or Mip1, Mip30 or Mip6 derivatives can be made by altering their respective sequence by substitutions, additions or deletions that provide for functionally equivalent molecules. Due to the degeneracy of nucleotide coding sequences, other DNA sequences that encode substantially the same amino acid sequence as a Mam or Mam-IP gene can be used in the practice of the present invention. These include but are not limited, to a nucleotide sequence comprising all or a portion of Mam, Mip1, Mip30, or Mip6 gene that is altered by the substitution of different codons that encode the same amino acid residue within the sequence, thus producing a silent change.

Likewise, Mam and Mam-IP derivatives of the invention include, but are not limited to, those containing, as a primary amino acid sequence, all or part of the amino acid sequence of Mam or a Mam-IP, including altered sequences in which functionally equivalent amino acid residues are substituted for residues within the sequence resulting in a silent change. For example, one or more amino acid residues within the sequence can be substituted by another amino acid of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

In a specific embodiment of the invention, proteins consisting of or comprising a fragment of Mam or Mam-IP consisting of at least 6 (continuous) amino acids of Mam or a Mam-IP are provided. In other embodiments, the fragment consists of at least about 10, 20, 30, 40, 50, 60, 70, 80, or 90 amino acids of Mam or a Mam-IP. In specific embodiments, such fragments are not larger than about 35, 50, 75, 100, 125, 150, 175, 200, 300, 400 or 500 amino acids. Derivatives or analogs of Mam and Mam-IPs, include, but are not limited to, molecules comprising regions that are substantially homologous to Mam or Mam-IPs, in various embodiments, by at least about 30%, 40%, 50%, 60%, 70%, 80%, 90% or 95% identity over an amino acid sequence of identical size or when compared to an aligned sequence in which the alignment is done by a computer homology program known in the art, or whose encoding nucleic acid is capable of hybridizing to the complement (e.g., the inverse complement) of a sequence encoding Mam or a Mam-IP under stringent, moderately stringent, or nonstringent conditions, as described infra.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87-2264-2268, modified as in Karlin and Altschul, 1993, Proc. Natl. Acad. Sci. USA 90:5873-5877. Such an algorithm is incorporated into the NBLAST and XBLAST programs of Altschul, et al., 1990, J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Altschul et al., 1997, supra). When utilizing BLAST, Gapped BLAST, and PSI-Blast programs, the default parameters of the respective programs (e.g., XBLAST and NBLAST) can be used (see http://www.ncbi.nlm.nih.gov). Another preferred, non-limiting example of a mathematical algorithm utilized for the comparison of sequences is the algorithm of Myers and Miller, 1988, CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The Mam, Mip1, Mip30 and Mip6 derivatives and analogs of the invention can be produced by various methods known in the art. The manipulations which result in their production can occur at the gene or protein level. For example, the cloned Mam or Mam-IP gene sequence can be modified by any of numerous strategies known in the art (Sambrook et al., 1989, Molecular Cloning, A Laboratory Manual, 2d Ed., Cold Spring Harbor Laboratory Press, Cold Spring Harbor, N.Y.). The sequences can be cleaved at appropriate sites with restriction endonuclease(s), followed by further enzymatic modification if desired, isolated, and ligated in vitro. In the production of a gene encoding a derivative or analog of Mam or a Mam-IP, care should be taken to ensure that the modified gene retains the original translational reading frame, uninterrupted by translational stop signals.

Additionally, the Mam and/or Mam-IP-encoding nucleotide sequence can be mutated in vitro or in vivo, to create and/or destroy translation, initiation, and/or termination sequences, or to create variations in coding regions and/or form new restriction endonuclease sites or destroy pre-existing ones, to facilitate further in vitro modification. Any technique for mutagenesis known in the art can be used, including but not limited to, chemical mutagenesis and in vitro site-directed mutagenesis (Hutchinson et al., 1978, J. Biol. Chem 253:6551-6558), use of TAB™ linkers (Pharmacia), etc.

Once a recombinant cell expressing Mam and/or a Mam-IP protein, or fragment or derivative thereof, is identified, the individual gene product or complex can be isolated and analyzed. This is achieved by assays based on the physical and/or functional properties of the protein or complex, including, but not limited to, radioactive labeling of the product followed by analysis by gel electrophoresis, immunoassay, cross-linking to marker-labeled product, etc.

The Mam:Mam complex, or Mam, Mip1, Mip30 or Mip6 protein, can be isolated and purified by standard methods known in the art (either from natural sources or recombinant host cells expressing the complexes or proteins), including but not restricted to column chromatography (e.g., ion exchange, affinity, gel exclusion, reversed-phase high pressure, fast protein liquid, etc.), differential centrifugation, differential solubility, or by any other standard technique used for the purification of proteins. Functional properties may be evaluated using any suitable assay known in the art.

Alternatively, once a Mam-IP or its derivative is identified, the amino acid sequence of the protein can be deduced from the nucleotide sequence of the chimeric gene from which it was encoded. As a result, the protein or its derivative can be synthesized by standard chemical methods known in the art (see, e.g., Hunkapiller et al., 1984, Nature 310: 105-111).

In a specific embodiment of the present invention, such Mam:Mam-IP complex, or Mam, Mip1, Mip30 or Mip6 protein, whether produced by recombinant DNA techniques, chemical synthesis methods, or by purification from native sources, include but are not limited to those containing as a primary amino acid sequence all or part of the amino acid sequences substantially as depicted in FIGS. 1-12 (SEQ ID NOS:2, 4, 6, 7 (Mam); SEQ ID NOS:9, 10, 11, 12, 13, 14, 15, 16 (Mip1); SEQ ID NO:18 (Mip30); and SEQ ID NO:20 (Mip6)), as well as fragments and other analogs and derivatives thereof, including proteins homologous thereto.

Manipulations of Mam and/or Mam-IP sequences may be made at the protein level. Included within the scope of the invention are derivatives of complexes of Mam and/or Mam-IP fragments, derivatives or analogs thereof that are differentially modified during or after translation, e.g., by glycosylation, acetylation, phosphorylation, amidation, prenylation, derivatization by known protecting/blocking groups, proteolytic cleavage, linkage to an antibody molecule or other cellular ligand, etc. Any of numerous chemical modifications may be carried out by known techniques, including but not limited to specific chemical cleavage by cyanogen bromide, trypsin, chymotrypsin, papain, V8 protease, NaBH₄, acetylation, formylation, oxidation, reduction, metabolic synthesis in the presence of tunicamycin, etc.

In specific embodiments, the Mam and/or Mam-IP sequences are modified to include a fluorescent label. In another specific embodiment, the Mam and/or the Mam-IP are modified to have a heterofunctional reagent; such heterofunctional reagents can be used to crosslink the protein to other members of the complex or to other Mam-IPs.

In addition, analogs and derivatives of Mam and/or a Mam-IP, or analogs and derivatives of Mam, Mip1, Mip30 or Mip6 protein, can be chemically synthesized. For example, a peptide corresponding to a portion of Mam and/or a Mam-IP, which comprises the desired domain or mediates the desired activity in vitro (e.g., Mam:Mam-IP complex formation) can be synthesized by use of a peptide synthesizer. Furthermore, if desired, non-classical amino acids or chemical amino acid analogs can be introduced as a substitution or addition into the Mam and/or a Mam-IP. Non-classical amino acids include but are not limited to the D-isomers of the common amino acids, amino isobutyric acid, 4-aminobutyric acid (4-Abu), 2-aminobutyric acid (2-Abu), 6-amino hexanoic acid (e-Ahx), 2-amino isobutyric acid (Aib), 3-amino propionoic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, C-methyl amino acids, N-methyl amino acids, and amino acid analogs in general. Furthermore, classical or non-classical amino acids can be D (dextrorotary) or L (levorotary).

In cases where natural products are suspected of being mutant or are isolated from new species, the amino acid sequence of Mam, or a Mam-IP isolated from the natural source, as well as those expressed in vitro, or from synthesized expression vectors in vivo or in vitro, can be determined from analysis of the DNA sequence, or alternatively, by direct sequencing of the isolated protein. Such analysis may be performed by manual sequencing or through use of an automated amino acid sequenator.

The Mam:Mam-IP complex, or Mam, Mip1, Mip30 or Mip6 protein, may also be analyzed by hydrophilicity analysis (Hopp and Woods, 1981, Proc. Natl. Acad. Sci. USA 78:3824-3828). A hydrophilicity profile can be used to identify the hydrophobic and hydrophilic regions of the proteins, and help predict their orientation to aid in the design of substrates for experimental manipulation, such as in binding experiments, antibody synthesis, etc. Secondary structural analysis can also be done to identify regions of the Mam and/or a Mam-IP that assume specific structures (Chou and Fasman, 1974, Biochemistry 13:222-223). Manipulation, translation, secondary structure prediction, hydrophilicity and hydrophobicity profiles, open reading frame prediction and plotting, and determination of sequence homologies, can be accomplished using computer software programs available in the art.

Other methods of structural analysis including but not limited to X-ray crystallography (Engstrom, 1974, Biochem. Exp. Biol. 11:7-13), mass spectroscopy and gas chromatography (see, Methods in Protein Science, J. Wiley and Sons, New York, 1997), and computer modeling (Fletterick and Zoller, eds., 1986, Computer Graphics and Molecular Modeling, In: Current Communications in Molecular Biology, Cold Spring Harbor Laboratory, Cold Spring Harbor Press, New York) can also be employed.

5.2 Identification and Isolation of Mip30 and Mip6 Genes

The present invention relates to the nucleotide sequences encoding a Mip30 or Mip6 protein. In specific embodiments, the Mip30, or Mip6 nucleic acid sequence comprises the sequence of SEQ ID NOS:17 or 19, respectively, or a portion thereof, or a nucleotide sequence encoding, in whole or in part, a Mip30 or Mip6 protein (e.g., a protein comprising the amino acid sequence of SEQ ID NOS:18 or 20, respectively, or a portion thereof). The invention provides purified nucleic acids consisting of at least 8 nucleotides (i.e., a hybridizable portion) of an Mip30, or Mip6 sequence. In other embodiments, the nucleic acids consist of at least about 25 (continuous) nucleotides, 50 nucleotides, 100 nucleotides, 150 nucleotides, or 200 nucleotides of a Mip30 or Mip6 gene sequence, or a full-length Mip30 or Mip6 gene sequence. In another embodiment, the nucleic acids are smaller than about 35, 200 or 500 nucleotides in length. Nucleic acids can be single or double stranded.

The invention also relates to nucleic acids hybridizable to or complementary to the foregoing sequences, in particular the invention provides the inverse complement to nucleic acids hybridizable to the foregoing sequences (i.e., the inverse complement of a nucleic acid strand has the complementary sequence running in reverse orientation to the strand so that the inverse complement would hybridize without mismatches to the nucleic acid strand; thus, for example, where the coding strand is hybridizable to a nucleic acid with no mismatches between the coding strand and the hybridizable strand, then the inverse complement of the hybridizable strand is identical to the coding strand). In specific aspects, nucleic acid molecules are provided which comprise a sequence complementary to (specifically are the inverse complement of) at least about 10, 25, 50, 100, or 200 nucleotides or the entire coding region of a Mip30 or Mip6 gene.

In a specific embodiment, a nucleic acid which is hybridizable to a Mip30 or Mip6 nucleic acid sequence (e.g., having sequence SEQ ID NOS:17 or 19, respectively), or to a nucleic acid sequence encoding a Mip30 or Mip6 protein derivative (or a complement of the foregoing), under conditions of low stringency, is provided. By way of example and not limitation, procedures using such conditions of low stringency are as follows (see also Shilo and Weinberg, 1981, Proc. Natl. Acad. Sci. USA 78:6789-6792): Filters containing DNA are pretreated for 6 hours at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe. Filters are incubated in hybridization mixture for 18-20 hours at 40° C., and then washed for 1.5 hours at 55° C. in a solution containing 2×SSC, 25 mM Tris-HCl (pH 7.4), 5 mM EDTA, and 0.1% SDS. The wash solution is replaced with fresh solution and incubated an additional 1.5 hours at 60° C. Filters are blotted dry and exposed for autoradiography. If necessary, filters are washed for a third time at 65-68° C. and reexposed to film. Other conditions of low stringency which may be used are well known in the art (e.g., as employed for cross-species hybridizations).

In another specific embodiment, a nucleic acid sequence which is hybridizable to an Mip30 or Mip6 nucleic acid sequence (or a complement of the foregoing) or to a nucleic acid sequence encoding a Mip30 or Mip6 derivative under conditions of high stringency is provided. By way of example and not limitation, procedures using such conditions of high stringency are as follows: Prehybridization of filters containing DNA is carried out for 8 hours to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 hours at 65° C. in prehybridization mixture containing 100 μg/ml denatured salmon sperm DNA and 5-20×10⁶ cpm of ³²P-labeled probe. Washing of filters is done at 37° C. for 1 hour in a solution containing 2×SSC, 0.01% PVP, 0.01% Ficoll, and 0.01% BSA. This is followed by a wash in 0.1×SSC at 50° C. for 45 minutes before autoradiography. Other conditions of high stringency which may be used are well known in the art.

In another specific embodiment, a nucleic acid sequence which is hybridizable to a Mip30 or Mip6 nucleic acid sequence or to a nucleic acid sequence encoding a Mip30 or Mip6 derivative (or a complement of the foregoing) under conditions of moderate stringency is provided. For example, but not limited to, procedures using such conditions of moderate stringency are as follows: Filters containing DNA are pretreated for 6 hours at 55° C. in a solution containing 6×SSC, 5× Denhardt's solution, 0.5% SDS and 100 μg/ml denatured salmon sperm DNA. Hybridizations are carried out in the same solution with 5-20×10⁶ cpm ³²P-labeled probe. Filters are incubated in hybridization mixture for 18-20 hours at 55° C., and then washed twice for 30 minutes at 60° C. in a solution containing 1×SSC and 0.1% SDS. Filters are blotted dry and exposed for autoradiography. Other conditions of moderate stringency which may be used are well-known in the art. Washing of filters is done at 37° C. for 1 hour in a solution containing 2×SSC, 0.1% SDS.

Nucleic acid molecules encoding derivatives and analogs of Mip30 or Mip6 proteins (see this Section, supra), or Mip30 or Mip6 antisense nucleic acids (see Section 5.6.9, infra) are additionally provided. As is readily apparent, as used herein, a “nucleic acid encoding a fragment or portion of a Mip30 or Mip6 protein” shall be construed as referring to a nucleic acid encoding only the recited fragment or portion of the Mip30 or Mip6 protein, and not the other contiguous portions of the Mip30 or Mip6 as a continuous sequence.

Within nucleotide sequences, potential open reading frames can be identified using the NCBI BLAST program ORF Finder available to the public. Because all known protein translation products are at least 60 amino acids or longer (Creighton, 1992, Proteins, 2^{nd} Ed., W.H. Freeman and Co., New York), only those ORFs potentially encoding a protein of 60 amino acids or more are considered. If an initiation methionine codon (ATG) and a translational stop codon (TGA, TAA, or TGA) are identified, then the boundaries of the protein are defined. Other potential proteins include any open reading frames that extend to the 5′end of the nucleotide sequence, in which case the open reading frame predicts the C-terminal or core portion of a longer protein. Similarly, any open reading frame that extends to the 3′ end of the nucleotide sequence predicts the N-terminal portion of a longer protein.

Any method available in the art can be used to obtain a full length (i.e., encompassing the entire coding region) cDNA clone encoding a Mip30 or Mip6 protein. In particular, the polymerase chain reaction (PCR) can be used to amplify sequences in silico from a cDNA library. Oligonucleotide primers that hybridize to sequences at the 3′ and 5′ termini of the identified sequences can be used as primers to amplify by PCR sequences from a nucleic acid sample (cDNA or DNA), preferably a cDNA library, from an appropriate source (e.g., the sample from which the initial cDNA library for the modified yeast two hybrid assay fusion population was derived).

PCR can be carried out, e.g., by use of a Perkin-Elmer Cetus thermal cycler and Taq polymerase. The DNA being amplified can include genomic DNA or cDNA sequences from any eukaryotic species. One can choose to synthesize several different degenerate primers, for use in the PCR reactions. It is also possible to vary the stringency of hybridization conditions used in priming the PCR reactions, to amplify nucleic acid homologs (e.g., to obtain Mip30 or Mip6 sequences from species other than humans, or to obtain human sequences with homology to Mip30 or Mip6) by allowing for greater or lesser degrees of nucleotide sequence similarity between the known nucleotide sequence and the nucleic acid homolog being isolated. For cross species hybridization, low stringency conditions are preferred. For same species hybridization, moderately stringent conditions are preferred.

After successful amplification of the nucleic acid containing all or a portion of the Mip30 or Mip6 sequence, that segment may be molecularly cloned and sequenced, and utilized as a probe to isolate a complete cDNA or genomic clone. This, in turn, will permit the determination of the gene's complete nucleotide sequence, the analysis of its expression, and the production of its protein product for functional analysis, as described infra. In this fashion, the nucleotide sequence of the entire Mip30 or Mip6 gene, as well as additional genes encoding a Mip30 or Mip6 protein or analog may be identified.

Any eukaryotic cell potentially can serve as the nucleic acid source for the molecular cloning of the Mip30 or Mip6 gene. The nucleic acids can be isolated from vertebrates, including mammalian, human, porcine, bovine, feline, avian, equine, canine, as well as additional primate sources, insects, plants, etc. The DNA may be obtained by standard procedures known in the art from cloned DNA (e.g., a DNA “library”), by chemical synthesis, by cDNA cloning, or by the cloning of genomic DNA, or fragments thereof, purified from the desired cell (see, for example, Sambrook et al., 1989, Molecular Cloning, A Laboratory Manual, 2d Ed., Cold Spring Harbor Laboratory Press, Cold Spring Harbor, N.Y.; Glover, D. M. (ed.), 1985, DNA Cloning: A Practical Approach, MRL Press, Ltd., Oxford, U.K. Vol. I, II). Clones derived from genomic DNA may contain regulatory and intronic DNA regions in addition to coding regions; clones derived from cDNA will contain only exon sequences. Whatever the source, the gene should be molecularly cloned into a suitable vector for propagation of the gene.

In the molecular cloning of the gene from genomic DNA, DNA fragments are generated, some of which will encode the desired gene. The DNA may be cleaved at specific sites using various restriction enzymes. Alternatively, one may use DNAse in the presence of manganese to fragment the DNA, or the DNA can be physically sheared, for example, by sonication. The linear DNA fragments can then be separated according to size by standard techniques, including but not limited to, agarose and/or polyacrylamide gel electrophoresis, and column chromatography.

Once the DNA fragments are generated, identification of the specific DNA fragment containing the desired gene may be accomplished in a number of ways. For example, a portion of the Mip30 or Mip6 gene (of any species) (e.g., a PCR amplification product obtained as described above, or an oligonucleotide having a sequence of a portion of the known nucleotide sequence) or its specific RNA, or a fragment thereof, may be purified and labeled, and the generated DNA fragments may be screened by nucleic acid hybridization to the labeled probe (Benton, W. and Davis, R., 1977, Science 196:180-182; Grunstein, M. And Hogness, D., 1975, Proc. Natl. Acad. Sci. U.S.A. 72:3961-3964). Those DNA fragments with substantial homology to the probe will hybridize. It is also possible to identify the appropriate fragment by restriction enzyme digestion(s) and comparison of fragment sizes with those expected according to a known restriction map if such is available, or by DNA sequence analysis and comparison to the known nucleotide sequence of Mip30 or Mip6. Further selection can be carried out on the basis of the properties of the gene. Alternatively, the presence of the gene may be detected by assays based on the physical, chemical, or immunological properties of its expressed product. For example, cDNA clones, or DNA clones which hybrid-select the proper mRNAs, can be selected which produce a protein that, e.g., has similar or identical electrophoretic migration, isolectric focusing behavior, proteolytic digestion maps, or antigenic properties or ability to bind Mam, as is known for Mip30 and Mip6. If an anti-Mip30 or anti-Mip6 antibody is available, the protein may be identified by binding of labeled antibody to the putatively Mip30 or Mip6 synthesizing clones, in an ELISA (enzyme-linked immunosorbent assay)-type procedure.

An alternative to isolating the Mip30 or Mip6 cDNA includes, but is not limited to, chemically synthesizing the gene sequence itself from a known sequence. Other methods are possible and within the scope of the invention.

The identified and isolated nucleic acids can then be inserted into an appropriate cloning vector. A large number of vector-host systems known in the art may be used. Possible vectors include, but are not limited to, plasmids or modified viruses, but the vector system must be compatible with the host cell used. Such vectors include, but are not limited to, bacteriophages such as lambda derivatives, or plasmids such as pBR322 or pUC plasmid derivatives or the pBluescript vector (Stratagene, La Jolla, Calif.). Insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically “polished” to ensure compatibility. Alternatively, any site desired may be produced by ligating nucleotide sequences (linkers) onto the DNA termini; these ligated linkers may comprise specific chemically synthesized oligonucleotides encoding restriction endonuclease recognition sequences. In an alternative method, the cleaved vector and the Mip30 or Mip6 gene may be modified by homopolymeric tailing. Recombinant molecules can be introduced into host cells via transformation, transfection, infection, electroporation, etc., so that many copies of the gene sequence are generated.

In an alternative method, the desired gene may be identified and isolated after insertion into a suitable cloning vector in a “shot gun” approach. Enrichment for the desired gene, for example, by size fractionation, can be done before insertion into the cloning vector.

In specific embodiments, transformation of host cells with recombinant DNA molecules that incorporate the isolated Mip30 or Mip6 gene, cDNA, or synthesized DNA sequence enables generation of multiple copies of the gene. Thus, the gene may be obtained in large quantities by growing transformants, isolating the recombinant DNA molecules from the transformants and, when necessary, retrieving the inserted gene from the isolated recombinant DNA.

The Mip30 or Mip6 nuclear acid sequence provided by the present invention includes those nucleotide sequences encoding substantially the same amino acid sequence as found in native Mip30 or Mip6 protein, and those encoded amino acid sequences with functionally equivalent amino acids, as well as those encoding other Mip30 or Mip6 derivatives or analogs, as described in Section 5.1, supra, for Mip30 and Mip6 derivatives and analogs.

5.3 Antibodies to Mam:Mam-IP Complexes, and Mip30 and Mip6 Proteins

According to the present invention, the Mam:Mam-IP complex (e.g., Mam complexed with Mip1, Mip30 or Mip6), or fragments, derivatives or homologs thereof, or Mip30 or Mip6 protein or fragments, homologs and derivatives thereof, may be used as immunogens to generate antibodies which immunospecifically bind such immunogens. Such antibodies include but are not limited to polyclonal, monoclonal, chimeric, and single chain antibodies, Fab fragments, and Fab expression libraries. In a specific embodiment, antibodies to complexes of human Mam and a human Mam-IP are produced. In another embodiment, complexes formed from fragments of a Mam and a Mam-IP, where the fragments contain the protein domain that interacts with the other member of the complex, are used as immunogens for antibody production. In another specific embodiment, Mip30 or Mip6 proteins or fragments, derivatives, or homologs thereof are used as immunogens.

Various procedures known in the art may be used for the production of polyclonal antibodies to a Mam:Mam-IP complex, or to a derivative or analog thereof, or to a Mip30 or Mip6 protein, or derivative, fragment or analog thereof.

For production of the antibody, various host animals can be immunized by injection with the native Mam:Mam-IP complex, or Mip30 or Mip6 protein, or a synthetic version, or a derivative of the foregoing, such as a cross-linked Mam:Mam-IP. Such host animals include but are not limited to rabbits, mice, rats, etc. Various adjuvants can be used to increase the immunological response, depending on the host species, and include but are not limited to Freund's (complete and incomplete), mineral gels such as aluminum hydroxide, surface active substances such as lysolecithin, pluronic polyols, polyanions, peptides, oil emulsions, dinitrophenol, and potentially useful human adjuvants such as bacille Calmette-Guerin (CG) and Corynebacterium parvum.

For preparation of monoclonal antibodies directed towards a Mam:Mam-IP complex or to a Mip30 or Mip6 protein, or derivatives, fragments or analogs thereof, any technique that provides for the production of antibody molecules by continuous cell lines in culture may be used. Two conceptually unique approaches are currently available for the production of human monoclonal antibodies—the ‘hybridoma’ technique, based on the fusion of antibody-producing B lymphocytes with plasmacytoma cells or lymphoblastoid cell lines; and the use of Epstein-Barr virus (EBV) to ‘immortalize’ antigen-specific human B lymphocytes. Such techniques include but are not restricted to the hybridoma technique originally developed by Kohler and Milstein (1975, Nature 256:495-497) (the cell lines are made by fusion of a mouse myeloma and mouse spleen cells from an immunised donor), the trioma technique (Rosen et al., 1977, Cell 11:139-147), the human B-cell hybridoma technique (Kozbor et al., 1983, Immunology Today 4:72), and the EBV hybridoma technique to produce human monoclonal antibodies (Cole et al., 1985, In: Monoclonal Antibodies and Cancer Therapy, Alan R. Liss, Inc., pp. 77-96). In this technique, as in the hybridoma procedure, it is important to use the blood lymphocytes of individuals who have previously been immunized with the antigens and have increased numbers of specific antibody-producing cells. The procedure involves two steps: (1) the enrichment of cells with receptors for the given antigen; and (2) ‘immortalization’ of these cells by EBV infection. In an additional embodiment of the invention, monoclonal antibodies can be produced in germ-free animals (See International Application No. PCT/US90/02545). According to the invention, human antibodies may be used and can be obtained by using human hybridomas (Cole et al., 1983, Proc. Natl. Acad. Sci. USA 80:2026-2030), or by transforming human B cells with EBV virus in vitro (Cole et al., 1985, In: Monoclonal Antibodies and Cancer Therapy, Alan R. Liss, Inc., pp. 77-96). In fact, according to the invention, techniques developed for the production of chimeric antibodies (Morrison et al., 1984, Proc. Natl. Acad. Sci. USA 81:6851-6855; Neuberger et al., 1984, Nature 312:604-608; Takeda et al., 1985, Nature 314:452-454) by splicing the genes from a mouse antibody molecule specific for the Mam:Mam-IP complex or Mip30 or Mip6 protein, together with genes from a human antibody molecule of appropriate biological activity, can be used; such antibodies are within the scope of this invention.

According to the invention, techniques described for the production of single chain antibodies (U.S. Pat. No. 4,946,778) can be adapted to produce Mam:Mam-IP complex-specific and Mip30 or Mip6 protein-specific single chain antibody. An additional embodiment of the invention utilizes techniques described for the construction of Fab expression libraries (Huse et al., 1989, Science 246:1275-1281) to allow rapid and easy identification of monoclonal Fab fragments with the desired specificity for the Mam:Mam-IP complex, or an individual Mip30 or Mip6 protein, derivative or analog. As reported by Huse et al., an Fab expression library was constructed from mRNA isolated from a mouse that had been immunized with the antigen NPN. The PCR amplification of messenger RNA isolated from spleen cells or hybridomas with oligonucleotides that incorporate restriction sites into the ends of the amplified product can be used to clone and express heavy and light chain sequences. Thus, the amplified fragments were cloned into a lambda phage vector in a predetermined reading frame for expression. The combinatorial library was constructed in two steps. In the first step, separate heavy and light chain libraries were constructed, and in the second step, these two libraries were used to construct a combinatorial library by crossing them at the EcoRI site. After ligation, only clones that resulted from combination of a right arm of light chain-containing clones and a left arm of heavy chain-containing clones reconstituted a viable phage. After ligation and packaging, 2.5×10⁷ clones were obtained. This is the combinatorial Fab expression library that was screened to identify clones having affinity for NPN. In an examination of approximately 500 recombinant phage, approximately 60 percent coexpressed light and heavy chain proteins. The light chain, heavy chain and Fab libraries were screened to determine whether they contained recombinant phage that expressed antibody fragments binding NPN. Non-human antibodies can be humanized by known methods (e.g., see U.S. Pat. No. 5,225,539).

Antibody fragments that contain the idiotypes of a Mam:Mam-IP complex or of a Mip30 or Mip6 protein can be generated by techniques known in the art. For example, such fragments include but are not limited to: the F(ab)₂ fragment which can be produced by pepsin digestion of the antibody molecule; the Fab′ fragments that can be generated by reducing the disulfide bridges of the F(ab)₂ fragment; the Fab fragments that can be generated by treating the antibody molecular with papain and a reducing agent; and Fv fragments.

In the production of antibodies, screening for the desired antibody can be accomplished by techniques known in the art, e.g., ELISA (enzyme-linked immunosorbent assay). To select antibodies specific to a particular domain of the Mam:Mam-IP complex, or Mip30 or Mip6 protein, one may assay generated hybridomas for a product that binds to the fragment of the Mam:Mam-IP complex, or the Mip30 or Mip6 protein, that contains such a domain. For selection of an antibody that specifically binds a Mam:Mam-IP complex but which does not specifically bind to the individual proteins of the Mam:Mam-IP complex, one can select on the basis of positive binding to the Mam:Mam-IP complex and a lack of binding to the individual Mam and Mam-IP proteins.

Antibodies specific to a domain of the Mam:Mam-IP complex are also provided, as are antibodies to specific domains of the Mip30 or Mip6 protein.

The foregoing antibodies can be used in methods known in the art relating to the localization and/or quantitation of a Mam:Mam-IP complex or of a Mip30 or Mip6 protein of the invention, e.g., for imaging these proteins, measuring levels thereof in appropriate physiological samples, in diagnostic methods, etc.

In another embodiment of the invention, anti-Mam:Mam-IP complex antibodies and fragments thereof, or anti-Mip30 or anti-Mpi6 antibodies or fragments thereof, containing the binding domain, are therapeutics, see Section 5.6 below.

5.4 Methods for Identifying Modulators of Notch Signal Transduction or Modulators of SUMO Conjugation Activity

The present invention is directed to methods of identifying a molecule that alters Notch signal transduction in a cell comprising contacting the cell with one or more candidate molecules; and measuring the amount of sumolation in the cell, wherein an increase or decrease in the amount of sumolation relative to said amount in a cell not so contacted with one or more of the candidate molecules indicates that the candidate molecules alter Notch signal transduction. The present invention is also directed to methods of identifying a molecule that alters Notch signal transduction in a cell comprising recombinantly expressing within the cell one or more candidate molecules; and measuring the amount of sumolation in the cell, wherein an increase or decrease in the amount of sumolation relative to said amount in a cell not so contacted with one or more of the candidate molecules indicates that the candidate molecules alter Notch signal transduction. The present invention is also directed to methods of identifying a molecule that alters Notch signal transduction in a cell comprising microinjecting into the cell one or more candidate molecules; and measuring the amount of sumolation in the cell, wherein an increase or decrease in the amount of sumolation relative to said amount in a cell not so contacted with one or more of the candidate molecules indicates that the candidate molecules alter Notch signal transduction.

The present invention is directed to methods of identifying a molecule that alters sumolation activity in a cell comprising contacting the cell with one or more candidate molecules; and measuring the amount of Notch signal transduction in the cell, wherein an increase or decrease in the amount of Notch signal transduction relative to said amount in a cell not so contacted with one or more of the candidate molecules indicates that the candidate molecules alter sumolation activity. Another method of identifying a molecule that alters sumolation in a cell comprises recombinantly expressing within the cell one or more candidate molecules; and measuring the amount of Notch signal transduction in the cell, wherein an increase or decrease in the amount of Notch signal transduction relative to said amount in a cell not so contacted with one or more of the candidate molecules indicates that the candidate molecules alter sumolation activity. Yet another method of identifying a molecule that alters sumolation activity in a cell comprises microinjecting into the cell one or more candidate molecules; and measuring the amount of Notch signal transduction in the cell, wherein an increase or decrease in the amount of Notch signal transduction relative to said amount in a cell not so contacted with one or more of the candidate molecules indicates that the candidate molecules alter sumolation activity.

Methods that can be used to carrying out the foregoing are commonly known in the art and/or those methods disclosed herein. The cells used in the methods of this embodiment can either endogenously or recombinantly express Mam, Mip1, Mip30 and/or Mip6, or a fragment, derivative or analog thereof. Recombinant expression of a Mam and/or Mam-IP is carried out by introducing the encoding nucleic acids into expression vectors and subsequently introducing the vectors into a cell to express the desired protein or simply introducing Mam and/or Mam-IP encoding nucleic acids into a cell for expression, as described in Section 5.2 or using procedures well known in the art. Nucleic acids encoding Mam and Mip1 from a number of species have been cloned and sequenced and their expression is well known in the art. Illustrative examples of Mam and Mip molecules are set forth in FIGS. 1 and 6. Expression can be from expression vectors or intrachromosomal. In a specific embodiment, standard human cell lines, such as HeLa cells and human kidney 293 cells, are employed in the screening assays.

Any method known to those of skill in the art for the insertion of Mam and/or Mam-IP-encoding DNA into a vector may be used to construct expression vectors for expressing Mam and/or Mam-IP, including those methods described in Section 5.2, supra. In addition, a host cell strain may be chosen which modulates the expression of Mam and/or Mam-IP, or modifies and processes the gene product in the specific fashion desired. Expression from certain promoters can be elevated in the presence of certain inducers; thus, expression of the desired protein may be controlled. Furthermore, different host cells have characteristic and specific mechanisms for the translational and post-translational processing and modification (e.g., glycosylation, cleavage) of proteins. Appropriate cell lines or host systems can be chosen to ensure the desired modification and processing of the expressed desired protein. For example, expression in a bacterial system can be used to produce an unglycosylated core protein product. Expression in yeast will produce a glycosylated product. Expression in mammalian cells can be used to ensure “native” glycosylation of a mammalian Mam and/or Mam-IP protein.

Sumolation, or SUMO conjugation activity, can be measured using methods well know in the art, e.g., by an increase or decrease in the conjugation of SUMO to target proteins. The total cellular complement of protein targets or specific protein targets can be analyzed. The SUMO protein can be introduced as a transgene in either an epitope-tagged form or an un-tagged form. Alternatively, the extent of endogenous SUMO conjugation activity can be assessed, e.g., using anti-SUMO antibodies, or by Western blot analysis in which the results would be amenable to quantification by densitometry. Further, since SUMO conjugation of a protein often influences the intracellular localization of the protein, an assay based upon the localization of a specific target protein can be used. Also, since SUMO conjugation of a protein often stabilizes the protein since SUMO competes with the same target lysine as ubiquitin, sumolation can be measured by measuring the stability, i.e., half-life, of the target protein, e.g., by Western blot analysis.

Notch signal transduction or Notch function can be measured using assays commonly known in the art, e.g., by the ability of Notch to activate transcription of a gene in the Enhancer of split complex, e.g., mγ, mδ, m5; or to activate transcription of vestigial, cut, or the HES1 gene. An in vitro transcription assay utilizing HES1 has been described (Wu et al., 2000, Nature Genetics 26:484-489; Jarriault et al., 1995, Nature 377:355-358). Thus, increased levels of mγ, mδ, m5, vestigial, cut or HES1 mRNA or protein indicates an increased level of Notch signal transduction or Notch function. Conversely, decreased levels of mγ, mδ, m5, vestigial, cut or HES1 mRNA or protein indicates a decreased level of Notch signal transduction or Notch function. Further, activation of Notch signal transduction results in the inhibition of differentiation of precursor cells. See, U.S. Pat. No. 5,780,300. Thus, Notch signal transduction can also be measured by assaying for differentiation of precursor cells. Maintenance of the differentiation state of the precursor cell indicates active Notch signal transduction. A change in the differentiation state of the precursor cell indicates inactive Notch signal transduction. Additionally, reporter constructs with a reporter gene under the control of a promoter containing a Notch-responsive promoter element can also be used to detect Notch signal transduction. For example, the EBNA2 response element from the TP-1 promoter can be used in such a reporter construct.

5.4.1 Candidate Molecules

Any molecule known in the art can be tested for its ability to modulate (increase or decrease) Notch signal transduction or sumolation activity as detected by a change in the ability of a cell to differentiate or a change in HES1 expression (for Notch signal transduction) or by a change in levels of sumolation of cellular proteins or amount thereof (for sumolation activity). By way of example, a change in the level of sumolation can be detected by detecting a change in the whether a test protein is conjugated to SUMO. For identifying a molecule that modulates Notch signal transduction or sumolation, candidate molecules can be directly provided to a cell or, in the case of candidate proteins, can be provided by providing their encoding nucleic acids under conditions in which the nucleic acids are recombinantly expressed to produce the candidate proteins within the cell.

This embodiment of the invention is well suited to screen chemical libraries for molecules which modulate, e.g., inhibit, antagonize, or agonize Notch signal transduction or sumolation activity. The chemical libraries can be peptide libraries, peptidomimetic libraries, chemically synthesized libraries, recombinant, e.g., phage display libraries, and in vitro translation-based libraries, other non-peptide synthetic organic libraries, etc.

Exemplary libraries are commercially available from several sources (ArQule, Tripos/PanLabs, ChemDesign, Pharmacopoeia). In some cases, these chemical libraries are generated using combinatorial strategies that encode the identity of each member of the library on a substrate to which the member compound is attached, thus allowing direct and immediate identification of a molecule that is an effective modulator. Thus, in many combinatorial approaches, the position on a plate of a compound specifies that compound's composition. Also, in one example, a single plate position may have from 1-20 chemicals that can be screened by administration to a well containing the interactions of interest. Thus, if modulation is detected, smaller and smaller pools of interacting pairs can be assayed for the modulation activity. By such methods, many candidate molecules can be screened.

Many diversity libraries suitable for use are known in the art and can be used to provide compounds to be tested according to the present invention. Alternatively, libraries can be constructed using standard methods. Chemical (synthetic) libraries, recombinant expression libraries, or polysome-based libraries are exemplary types of libraries that can be used.

The libraries can be constrained or semirigid (having some degree of structural rigidity), or linear or nonconstrained. The library can be a cDNA or genomic expression library, random peptide expression library or a chemically synthesized random peptide library, or non-peptide library. Expression libraries are introduced into the cells in which the assay occurs, where the nucleic acids of the library are expressed to produce their encoded proteins.

In one embodiment, peptide libraries that can be used in the present invention may be libraries that are chemically synthesized in vitro. Examples of such libraries are given in Houghten et al., 1991, Nature 354:84-86, which describes mixtures of free hexapeptides in which the first and second residues in each peptide were individually and specifically defined; Lam et al., 1991, Nature 354:82-84, which describes a “one bead, one peptide” approach in which a solid phase split synthesis scheme produced a library of peptides in which each bead in the collection had immobilized thereon a single, random sequence of amino acid residues; Medynski, 1994, Bio/Technology 12:709-710, which describes split synthesis and T-bag synthesis methods; and Gallop et al., 1994, J. Medicinal Chemistry 37(9):1233-1251. Simply by way of other examples, a combinatorial library may be prepared for use, according to the methods of Ohlmeyer et al., 1993, Proc. Natl. Acad. Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; or Salmon et al., 1993, Proc. Natl. Acad. Sci. USA 90:11708-11712. PCT Publication No. WO 93/20242 and Brenner and Lerner, 1992, Proc. Natl. Acad. Sci. USA 89:5381-5383 describe “encoded combinatorial chemical libraries,” that contain oligonucleotide identifiers for each chemical polymer library member.

In a preferred embodiment, the library screened is a biological expression library that is a random peptide phage display library, where the random peptides are constrained (e.g., by virtue of having disulfide bonding).

Further, more general, structurally constrained, organic diversity (e.g., nonpeptide) libraries, can also be used. By way of example, a benzodiazepine library (see e.g., Bunin et al., 1994, Proc. Natl. Acad. Sci. USA 91:4708-4712) may be used.

Conformationally constrained libraries that can be used include but are not limited to those containing invariant cysteine residues which, in an oxidizing environment, cross-link by disulfide bonds to form cystines, modified peptides (e.g., incorporating fluorine, metals, isotopic labels, are phosphorylated, etc.), peptides containing one or more non-naturally occurring amino acids, non-peptide structures, and peptides containing a significant fraction of γ-carboxyglutamic acid.

Libraries of non-peptides, e.g., peptide derivatives (for example, that contain one or more non-naturally occurring amino acids) can also be used. One example of these are peptoid libraries (Simon et al., 1992, Proc. Natl. Acad. Sci. USA 89:9367-9371). Peptoids are polymers of non-natural amino acids that have naturally occurring side chains attached not to the alpha carbon but to the backbone amino nitrogen. Since peptoids are not easily degraded by human digestive enzymes, they are advantageously more easily adaptable to drug use. Another example of a library that can be used, in which the amide functionalities in peptides have been permethylated to generate a chemically transformed combinatorial library, is described by Ostresh et al., 1994, Proc. Natl. Acad. Sci. USA 91:11138-11142).

The members of the peptide libraries that can be screened according to the invention are not limited to containing the 20 naturally occurring amino acids. In particular, chemically synthesized libraries and polysome based libraries allow the use of amino acids in addition to the 20 naturally occurring amino acids (by their inclusion in the precursor pool of amino acids used in library production). In specific embodiments, the library members contain one or more non-natural or non-classical amino acids or cyclic peptides. Non-classical amino acids include but are not limited to the D-isomers of the common amino acids, α-amino isobutyric acid, 4-aminobutyric acid, Abu, 2-amino butyric acid; γ-Abu, ε-Ahx, 6-amino hexanoic acid; Aib, 2-amino isobutyric acid; 3-amino propionic acid; ornithine; norleucine; norvaline, hydroxyproline, sarcosine, citrulline, cysteic acid, t-butylglycine, t-butylalanine, phenylglycine, cyclohexylalanine, β-alanine, designer amino acids such as β-methyl amino acids, Cα-methyl amino acids, Nα-methyl amino acids, fluoro-amino acids and amino acid analogs in general. Furthermore, the amino acid can be D (dextrorotary) or L (levorotary).

In a specific embodiment, fragments and/or analogs of Mam or Mip1, especially peptidomimetics, are screened for activity as competitive or non-competitive inhibitors of Notch signal transduction or sumolation activity.

In another embodiment of the present invention, combinatorial chemistry can be used to identify modulators of Notch signal transduction or sumolation activity. Combinatorial chemistry is capable of creating libraries containing hundreds of thousands of compounds, many of which may be structurally similar. While high throughput screening programs are capable of screening these vast libraries for affinity for known targets, new approaches have been developed that achieve libraries of smaller dimension but which provide maximum chemical diversity. (See e.g., Matter, 1997, Journal of Medicinal Chemistry 40:1219-1229).

One method of combinatorial chemistry, affinity fingerprinting, has previously been used to test a discrete library of small molecules for binding affinities for a defined panel of proteins. The fingerprints obtained by the screen are used to predict the affinity of the individual library members for other proteins or receptors of interest (in the instant invention, e.g., Mip1). The fingerprints are compared with fingerprints obtained from other compounds known to react with the protein of interest to predict whether the library compound might similarly react. For example, rather than testing every ligand in a large library for interaction with Mip1, only those ligands having a fingerprint similar to other compounds known to have that activity could be tested. (See, e.g., Kauvar et al., 1995, Chemistry and Biology 2:107-118; Kauvar, 1995, Affinity fingerprinting, Pharmaceutical Manufacturing International. 8:25-28; and Kauvar, Toxic-Chemical Detection by Pattern Recognition in New Frontiers in Agrochemical Immunoassay, D. Kurtz. L. Stanker and J. H. Skerritt. Editors, 1995, AOAC: Washington, D.C., 305-312).

Kay et al., 1993, Gene 128:59-65 (Kay) discloses a method of constructing peptide libraries that encode peptides of totally random sequence that are longer than those of any prior conventional libraries. The libraries disclosed in Kay encode totally synthetic random peptides of greater than about 20 amino acids in length. Such libraries can be advantageously screened to identify modulators of Notch signal transduction or sumolation activity. (See also U.S. Pat. No. 5,498,538 dated Mar. 12, 1996; and PCT Publication No. WO 94/18318 dated Aug. 18, 1994).

A comprehensive review of various types of peptide libraries can be found in Gallop et al., 1994, J. Med. Chem. 37:1233-1251.

5.5 Diagnostic, Prognostic, and Screening Uses of Mam:Mam-IP Complexes and Nucleic Acids, and Mip30 and Mip6 Proteins and Nucleic Acids

Mam:Mam-IP complexes (particularly Mam complexed with one of the following: Mip1, Mip30 or Mip6) may be markers of normal physiological processes including, but not limited to, the physiological processes including signal transduction, cell fate and differentiation and mitotic events, such as chromosomal segregation, and thus have diagnostic utility. Further, definition of particular groups of patients with elevations or deficiencies of a Mam:Mam-IP complex, or a Mip30 or Mip6 protein, can lead to new classifications of diseases, furthering diagnostic ability.

Detecting levels of Mam:Mam-IP complexes, or individual proteins that have been shown to form complexes with Mam, or the Mip30 or Mip6 proteins; or detecting levels of mRNAs encoding components of the Mam:Mam-IP complexes, or mRNAs encoding the Mip30 or Mip6 protein, may be used in prognosis, to follow the course of disease state, to follow therapeutic response, etc.

Mam:Mam-IP complexes and the individual components of the Mam:Mam-IP complexes (e.g., Mam, Mip1, Mip30, Mip6), and derivatives, analogs and subsequences thereof; Mam and/or Mam-IP, or Mip30 or Mip6 nucleic acids (and sequences complementary thereto); anti-Mam:Mam-IP complex antibodies and antibodies directed against the individual components that can form Mam:Mam-IP complexes; and anti-Mip30 or anti-Mip6 antibodies, have uses in diagnostics. Such molecules can be used in assays, such as immunoassays, to detect, prognose, diagnose, or monitor various conditions, diseases, and disorders, and treatment thereof, characterized by aberrant levels of Mam:Mam-IP complexes, or by aberrant levels of Mip30 or Mip6 protein.

In particular, such an immunoassay is carried out by a method comprising contacting a sample derived from a patient with an anti-Mam:Mam-IP complex antibody, or an anti-Miup30 or anti-Mip6 antibody under conditions such that immunospecific binding can occur, and detecting or measuring the amount of any immunospecific binding by the antibody. In a specific aspect, such binding of antibody, in tissue sections, can be used to detect aberrant Mam:Mam-IP complex formation, or aberrant Mip30 or Mip6 protein localization, or aberrant (e.g., high, low or absent) levels of Mam:Mam-IP complex or complexes, or aberrant levels of Mip30 or Mip6 protein. In a specific embodiment, an antibody against a Mam:Mam-IP complex can be used to assay a patient tissue or serum sample for the presence of the Mam:Mam-IP complex, where an aberrant level of the Mam:Mam-IP complex is an indication of a disease condition. In another embodiment, an antibody against Mip30 or Mip6 can be used to assay a patient tissue or serum sample for the presence of Mip30 or Mip6 where an aberrant level of Mip30 or Mip6 is an indication of a disease condition. By “aberrant levels” is meant increased or decreased levels relative to that present, or a standard level representing that present, in an analogous sample from a portion of the body or from a subject not having the disorder.

The immunoassays which can be used include but are not limited to competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, and protein A immunoassays, to name but a few.

Nucleic acids encoding the components of the Mam:Mam-IP complexes (e.g., Mam, Mip1, Mip30 or Mip6) and nucleic acids encoding a Mip30 or Mip6 protein, and related nucleotide sequences and subsequences, including complementary sequences, can also be used in hybridization assays. The Mam and/or Mam-IP nucleotide sequence, or a subsequence thereof, comprising about at least 8 nucleotides, can be used as hybridization probes. Hybridization assays can be used to detect, prognose, diagnose, or monitor conditions, disorders, or disease states associated with aberrant levels of the mRNAs encoding the components of a Mam:Mam-IP complex, or a Mip30 or Mip6 protein, as described supra. In particular, such a hybridization assay is carried out by a method comprising contacting a sample containing nucleic acid with a nucleic acid probe capable of hybridizing to Mam or a Mam-IP DNA or RNA, under conditions such that hybridization can occur, and detecting or measuring any resulting hybridization. In a preferred aspect, the hybridization assay is carried out using nucleic acid probes capable of hybridizing to Mam and to a binding partner of Mam to measure concurrently the expression of both members of a Mam:Mam-IP complex. In another preferred embodiment, the expression of mRNAs encoding Mip30 or Mip6 is measured.

In specific embodiments, diseases and disorders involving or characterized by aberrant levels of Mam:Mam-IP complexes (e.g., complexes of Mam with Mip1, Mip30 or Mip6 protein) can be diagnosed, or their suspected presence can be screened for, or a predisposition to develop such disorders can be detected, by detecting aberrant levels of a Mam:Mam-IP complex, or un-complexed Mam and/or a Mam-IP protein or nucleic acids or functional activity, including but not restricted to, binding to an interacting partner, or by detecting mutations in Mam and/or in a Mam-IP RNA, DNA or protein (e.g., translocations, truncations, changes in nucleotide or amino acid sequence relative to wild-type Mam and/or Mam-IP) that cause increased or decreased expression or activity of a Mam:Mam-IP complex and/or Mam and/or protein that binds to Mam. Such diseases and disorders include but are not limited to those described in Section 5.6 and its subsections.

By way of example, levels of a Mam:Mam-IP complex or the individual components of a Mam:Mam-IP complex can be detected by immunoassay; levels of Mam and/or of Mam-IP mRNA can be detected by hybridization assays (e.g., Northern blots, dot blots); binding of Mam or to a Mam-IP can be measured by binding assays commonly known in the art, translocations and point mutations in Mam and/or in genes encoding a Mam-IP can be detected by Southern blotting, RFLP analysis, PCR using primers that preferably generate a fragment spanning at least most of the Mam and/or Mam-IP gene, sequencing of the Mam and/or Mam-IP genomic DNA or cDNA obtained from the patient, etc.

Assays well known in the art (e.g., assays described above such as immunoassays, nucleic acid hybridization assays, activity assays, etc.) can be used to determine whether one or more particular Mam:Mam-IP complexes are present at either increased or decreased levels, or are absent, in samples from patients suffering from a particular disease or disorder, or having a predisposition to develop such a disease or disorder as compared to the levels in samples from subjects not having such a disease or disorder.

Additionally, these assays can be used to determine whether the ratio of the Mam:Mam-IP complex to the un-complexed components of the Mam:Mam-IP complex, i.e., Mam and/or the specific Mam-IP in the complex of interest, is increased or decreased in samples from patients suffering from a particular disease or disorder, or having a predisposition to develop such a disease or disorder, as compared to the ratio in samples from subjects not having such a disease or disorder.

In the event that levels of one or more particular Mam:Mam-IP complexes are determined to be increased in patients suffering from a particular disease or disorder, or having a predisposition to develop such a disease or disorder, then the particular disease or disorder or predisposition for a disease or disorder can be diagnosed, have prognosis defined for, be screened for, or be monitored by detecting increased levels of the one or more Mam:Mam-IP complexes, the mRNA that encodes the members of the one or more particular Mam:Mam-IP complexes, or Mam:Mam-IP complex functional activity.

Accordingly, in a specific embodiment of the invention, diseases and disorders involving increased levels of one or more Mam:Mam-IP complexes can be diagnosed, or their suspected presence can be screened for, or a predisposition to develop such disorders can be detected, by detecting increased levels of the one or more Mam:Mam-IP complexes, the mRNA encoding both members of the complex, or complex functional activity, or by detecting mutations in Mam or the Mam-IP (e.g., translocations in nucleic acids, truncations in the gene or protein, changes in nucleotide or amino acid sequence relative to wild-type Mam or Mam-IP) that stabilize or increase Mam:Mam-IP complex formation.

In the event that levels of one or more particular Mam:Mam-IP complexes are determined to be decreased in patients suffering from a particular disease or disorder or having a predisposition to develop such a disease or disorder, then the particular disease or disorder or predisposition for a disease or disorder can be diagnosed, have its prognosis determined, be screened for, or be monitored by detecting decreased levels of the one or more Mam:Mam-IP complexes, the mRNA that encodes the members of the particular one or more Mam:Mam-IP complexes, or Mam:Mam-IP complex functional activity.

Accordingly, in a specific embodiment of the invention, diseases and disorders involving decreased levels of one or more Mam:Mam-IP complexes can be diagnosed, or their suspected presence can be screened for, or a predisposition to develop such disorders can be detected, by detecting decreased levels of the one or more Mam:Mam-IP complexes, the mRNA encoding the members of the one or more complexes, or complex functional activity, or by detecting mutations in Mam or the Mam-IP (e.g., translocations in nucleic acids, truncations in the gene or protein, changes in nucleotide or amino acid sequence relative to wild-type Mam or the Mam-IP) that inhibit or reduce Mam:Mam-IP complex formation.

In another specific embodiment, diseases and disorders involving aberrant expression of a Mip30 or Mip6 protein are diagnosed, or their suspected presence can be screened for, or a predisposition to develop such disorders can be detected, by detecting aberrant levels of a Mip30 or Mip6 protein, or mRNA, or functional activity, or by detecting mutations in a Mip30 or Mip6 protein or mRNA or DNA (e.g., translocations in nucleic acids, truncations in the gene or protein, changes in nucleotide or amino acid sequence relative to wild-type Mip30 or Mip6) that cause aberrant expression or activity of Mip30 or Mip6 protein. Such diseases and disorders include but are not limited to those described infra, Section 5.6. By way of example, levels of Mip30 or Mip6 mRNA or protein, Mam binding activity, or the presence of translocations or point mutations, can be determined as described above.

Assays well known in the art (e.g., assays described above such as immunoassays, nucleic acid hybridization assays, activity assays, etc.) can be used to determine whether Mip30 or Mip6 are present at either increased or decreased levels, or are absent, in samples from patients suffering from a particular disease or disorder, or having a predisposition to develop such a disease or disorder, as compared to the levels in samples from subjects not having such a disease or disorder.

In the event that levels of Mip30 or Mip6 are determined to be increased in patients suffering from a particular disease or disorder, or having a predisposition to develop such a disease or disorder, then the particular disease or disorder or predisposition for a disease or disorder can be diagnosed, have its prognosis determined, be screened for, or be monitored by detecting increased levels of Mip30 or Mip6 protein or mRNA, or Mip30 or Mip6 functional activity (e.g., binding to Mam).

Accordingly, in a specific embodiment of the invention, diseases and disorders involving increased levels of a Mip30 or Mip6 protein can be diagnosed, or their suspected presence can be screened for, or a predisposition to develop such disorders can be detected, by detecting increased levels of a Mip30 or Mip6 protein or encoding nucleic acids, or Mip30 or Mip6 functional activity, or by detecting mutations in Mip30 or Mip6 (e.g., translocations in nucleic acids, truncations in the gene or protein, changes in nucleotide or amino acid sequence relative to wild-type Mip30 or Mip6) that enhance Mip30 or Mip6 stability or functional activity.

In the event that levels of Mip30 or Mip6 are determined to be decreased in patients suffering from a particular disease or disorder or having a predisposition to develop such a disease or disorder, then the particular disease or disorder or predisposition for a disease or disorder can be diagnosed, or prognosis determined, be screened for, or be monitored by detecting decreased levels of the Mip30 or Mip6 proteins or nucleic acids, or Mip30 or Mip6 functional activity.

Accordingly, in a specific embodiment of the invention, diseases and disorders involving decreased levels of Mip30 or Mip6 can be diagnosed, or their suspected presence can be screened for, or a predisposition to develop such disorders can be detected, by detecting decreased levels of Mip30 or Mip6 protein or nucleic acids, or Mip30 or Mip6 functional activity, or by detecting mutations in Mip30 or Mip6 (e.g., translocations in nucleic acids, truncations in the gene or protein, changes in nucleotide or amino acid sequence relative to wild-type Mip30 or Mip6) that destabilize or reduce Mip30 or Mip6 functional activity.

The use of detection techniques, especially those involving antibodies against Mam:Mam-IP complexes, or against a Mip30 or Mip6 protein, provides a method of detecting specific cells that express the complex or protein. Using such assays, specific cell types can be defined in which one or more particular Mam:Mam-IP complex, or Mip30 or Mip6 protein, is expressed, and the presence of the complex or protein can be correlated with cell viability.

Also embodied are methods to detect a Mam:Mam-IP complex, or a Mip30 or Mip6 protein, in cell culture models that express particular Mam:Mam-IP complexes or Mip30 or Mip6 proteins, or derivatives thereof, for the purpose of characterizing or preparing Mam:Mam-IP complexes, or Mip30 or Mip6 proteins for harvest. This embodiment includes cell sorting of prokaryotes such as, but not restricted to, bacteria (Davey and Kell, 1996, Microbiol. Rev. 60: 641-696), primary cultures and tissue specimens from eukaryotes, including mammalian species such as human (Steele et al., 1996, Clin. Obstet. Gynecol 39:801-813), and continuous cell cultures (Orfao and Ruiz-Arguelles, 1996, Clin. Biochem. 29:5-9). Such isolations can also be used as methods of diagnosis, described supra.

Kits for diagnostic use are also provided that comprise in one or more containers an anti-Mam:Mam-IP complex antibody or an anti-Mip30 or anti-Mip6 antibody, and, optionally, a labeled binding partner to the antibody. Alternatively, the anti-Mam:Mam-IP complex antibody, or anti-Mip30 or anti-Mip6 antibody, can be labeled with a detectable marker, e.g., a chemiluminescent, enzymatic, fluorescent, or radioactive moiety. A kit is also provided that comprises in one or more containers a nucleic acid probe capable of hybridizing to Mam and/or a Mam-IP (e.g., Mip1, Mip30, Mip6) mRNA. In a specific embodiment, a kit can comprise in one or more containers a pair of primers (e.g., each in the size range of about 6-30 nucleotides) that are capable of priming amplification [e.g., by polymerase chain reaction (see e.g., Innis et al., 1990, PCR Protocols, Academic Press, Inc., San Diego, Calif.), ligase chain reaction (see EP 320,308), use of Qβ replicase, cyclic probe reaction, or other methods known in the art], under appropriate reaction conditions of at least a portion of a Mam and/or a Mam-IP, or an Mip30 or Mip6 nucleic acid sequence. A kit can optionally further comprise in a container a predetermined amount of a purified Mam:Mam-IP complex, Mam and/or a Mam-IP, or a Mip30 or Mip6 protein or an encoding nucleic acid molecule thereof, e.g. for use as a standard or control.

5.6 Therapeutic Uses of Mam:Mam-IP Complexes and Mip30 and Mip6

5.6.1 Therapeutic Uses of Mam and Mam-Interactants

The invention provides for treatment or prevention of various diseases and disorders by administration of a therapeutic compound (termed herein “Therapeutic”). Such “Therapeutics” include but are not limited to: Mam:Mam-IP complexes (e.g., Mam complexed with Mip1, Mip30 or Mip6), Mam and the individual Mam-IP proteins and analogs and derivatives (including fragments) of the foregoing (e.g., as described herein above); antibodies there to (as described herein above); nucleic acids encoding Mam and/or a Mam-IP, and analogs or derivatives thereof (e.g., as described herein above); Mam and/or Mam-IP antisense nucleic acids, and Mam:Mam-IP complex and Mip30 and Mip6 modulators (i.e., inhibitors, agonists and antagonists).

As reviewed in Section 2, supra, Mam is centrally implicated in physiological processes, including but not limited to, signal transduction, and cell fate and differentiation. Likewise, Mam has been strongly implicated in pathological conditions, including but not limited to, and cancer. The Mam interactant Mip1, described in the present invention, is involved in mitosis, telomere regulation, and chromosome segregation, see section 2, supra.

Disorders of cell cycle progression, cell differentiation, and transcriptional control, including cancer and tumorigenesis and tumor progression can involve Mam and particularly the interactants Mip1, Mip30 and Mip6. The effect of the Mip1 protein on tumorigenesis may be due to the involvement of aberrant mitotic events in cancer.

Interactants Mip30 and Mip6 show no overall homologies to known proteins. However, Mip30 contains seven C2H2 zinc-finger repeats, which may be involved in protein-protein interactions, a HMG-1 and HMG-Y DNA-binding domain (A+T-hook), and a bipartite nuclear localization signal. The only identifiable motif in the Mip6 protein is a bipartite nuclear localization signal (amino acids 420-437).

5.6.2 Treatment of Diseases and Disorders with Increased Mam:Mam-IP Complexes

A wide range of cell diseases affected by intracellular signal transduction, and chromosome segregation can be treated or prevented by administration of a Therapeutic that modulates (i.e., inhibits, antagonizes, enhances or promotes) Mam:Mam-IP complex activity. All of these disorders can be treated or prevented by administration of a Therapeutic that modulates (i.e., inhibits, antagonizes, enhances or promotes) Mam:Mam-IP complex activity, or modulates Mip30 or Mip6 activity.

Diseases or disorders associated with aberrant levels of Mam:Mam-IP complex levels or activity, or aberrant levels of Mip30 or Mip6, may be treated by administration of a Therapeutic that modulates Mam:Mam-IP complex formation or activity, or Mip30 or Mip6 activity. In a specific embodiment, the activity or level of Mam is modulated by administration of a Mam-IP. In another specific embodiment, the activity or level of a Mam-IP is modulated by administration of Mam.

5.6.2.1 Antagonizing the Complex Formation or Activity

Diseases and disorders characterized by increased (relative to a subject not suffering from the disease or disorder) Mam:Mam-IP levels or activity, or increased Mip30 or Mip6 levels or activity, can be treated with Therapeutics that antagonize (i.e., reduce or inhibit) Mam:Mam-IP complex formation or activity, or Mip30 or Mip6 levels or activity. Therapeutics that can be used, include but are not limited to, Mam or a Mam-IP, or analogs, derivatives or fragments thereof; anti-Mam:Mam-IP complex antibodies (e.g., antibodies specific for Mam:Mip1, Mam:Mip30, Mam:Mip6 complexes), and anti-Mip30 or anti-Mip6 antibodies, fragments and derivatives thereof containing the binding region thereof; nucleic acids encoding Mam or a Mam-IP; concurrent administration of Mam and Mam-IP antisense nucleic acids, or Mip30 or Mip6 antisense nucleic acids, or Mam and/or Mam-IP, or Mip30 or Mip6 nucleic acids that are dysfunctional (e.g., due to a heterologous (non-Mam and/or non-Mam-IP, or non-Mip30 or non-Mip6) insertion within the coding sequences of the Mam coding sequences)) that are used to “knockout” endogenous Mam and/or Mam-IP function by homologous recombination (see, e.g., Capecchi, 1989, Science 244:1288-1292).

In a specific embodiment of the invention, a nucleic acid containing a portion of a Mam and/or a Mam-IP gene in which the Mam and/or Mam-IP sequences flank (are both 5′ and 3′ to) a different gene sequence, is used as a Mam and/or a Mam-IP antagonist, or to promote Mam and/or Mam-IP inactivation by homologous recombination (see also Koller and Smithies, 1989, Proc. Natl. Acad. Sci. USA 86:8932-8935, Zijlstra et al., 1989, Nature 342:435-438). Additionally, mutants or derivatives of a first Mam-IP protein that have greater affinity for Mam than a second Mam-IP may be administered to compete with the second Mam-IP protein for Mam binding, thereby reducing the levels of Mam complexes with the second Mam-IP. Other Therapeutics that inhibit Mam:Mam-IP complex or Mip30 or Mip6 function can be identified by use of known convenient in vitro assays, e.g., based on their ability to inhibit Mam:Mam-IP binding or as described in Section 5.8 infra.

In specific embodiments, Therapeutics that antagonize Mam:Mam-IP complex formation or activity, or a Mip30 or Mip6 activity, are administered therapeutically (including prophylactically): (1) in diseases or disorders involving an increased (relative to normal or desired) level of Mam:Mam-IP complex, or a Mip30 or Mip6 protein, for example, in patients where a Mam:Mam-IP complex or a Mip30 or Mip6 protein is overactive or overexpressed; or (2) in diseases or disorders wherein in vitro (or in vivo) assays (see infra) indicate the utility of a Mam:Mam-IP complex or Mip30 or Mip6 antagonist administration. Increased levels of Mam:Mam-IP complexes or increased Mip30 or Mip6 protein levels, can be readily detected, e.g., by quantifying protein and/or RNA, by obtaining a patient tissue sample (e.g., from biopsy tissue) and assaying it in vitro for RNA or protein levels, structure and/or activity of the expressed Mam:Mam-IP complex (or the Mam and Mam-IP mRNA), or the Mip30 or Mip6 protein or mRNA levels. Many methods standard in the art can be thus employed, including but not limited to: immunoassays to detect and/or visualize Mam:Mam-IP complexes, or Mip30 or Mip6 protein (e.g., Western blot, immunoprecipitation followed by sodium dodecyl sulfate polyacrylamide gel electrophoresis, immunocytochemistry, etc.) and/or hybridization assays to detect concurrent expression of Mam and a Mam-IP, or individual Mip30 or Mip6 mRNA (e.g., Northern blot assays, dot blots, in situ hybridization, etc.).

5.6.2.2 Reducing the Complex Expression

A more specific embodiment includes methods of reducing Mam:Mam-IP complex expression (i.e., the expression of the two components of the Mam:Mam-IP complex and/or formation of the complex), or reducing Mip30 or Mip6 expression, by targeting mRNAs that express the protein moieties. RNA therapeutics currently fall within three classes, antisense species, ribozymes, or RNA aptamers (Good et al., 1997, Gene Therapy 4:45-54).

Antisense oligonucleotides have been the most widely used. By way of example, but not for limitation, antisense oligonucleotide methodology to reduce Mam:Mam-IP complex formation is presented below in Subsection 5.6.8. Ribozyme therapy involves the administration, induced expression, etc., of small RNA molecules with enzymatic ability to cleave, bind, or otherwise inactivate specific RNAs to reduce or eliminate expression of particular proteins (Grassi and Marini, 1996, Annals of Medicine 28:499-510, Gibson, 1996, Cancer and Metastasis Reviews 15:287-299). At present, the design of hairpin and hammerhead RNA ribozymes is necessary to specifically target a particular mRNA, such as the mRNA encoding Mam. RNA aptamers are specific RNA ligands for proteins, such as for Tat and Rev RNA (Good et al., 1997, Gene Therapy 4:45-54) that can specifically inhibit their translation. Aptamers specific for Mam or a Mam-IP can be identified by many methods well known in the art, for example but not limited to the protein-protein interaction assay described in Section 5.8.1 infra.

In another embodiment, the activity or level of Mam is reduced by administration of a Mam-IP, or a nucleic acid that encodes a Mam-IP, or antibody that immunospecifically binds to a Mam-IP, or a fragment or a derivative of the antibody containing the binding domain thereof. Additionally, the level or activity of a Mam-IP may be reduced by administration of a Mam or a Mam-IP nucleic acid, or an antibody that immunospecifically binds Mam, or a fragment or derivative of the antibody containing the binding domain thereof.

In another aspect of the invention, diseases or disorders associated with increased levels of Mam or a particular Mam-IP (e.g., Mip1, Mip30, Mip6) may be treated or prevented by administration of a Therapeutic that increases Mam:Mam-IP complex formation, if the complex formation acts to reduce or inactivate Mam or the particular Mam-IP through Mam:Mam-IP complex formation. Such diseases or disorders can be treated or prevented by administration of one member of the Mam:Mam-IP complex, including mutants of a member of the Mam:Mam-IP that have increased affinity for the other member of the Mam:Mam-IP complex (to cause increased complex formation), administration of antibodies or other molecules that stabilize the Mam:Mam-IP complex, etc.

5.6.3 Treatment of Diseases and Disorders Associated with Underexpressed Mam:Mam-IP Complexes

Diseases and disorders associated with underexpression of a Mam:Mam-IP complex, or Mam or a particular Mam-IP, are treated or prevented by administration of a Therapeutic that promotes (i.e., increases or supplies) Mam:Mam-IP complexes or function. Examples of such a Therapeutic include but are not limited to Mam:Mam-IP complexes and derivatives, analogs and fragments thereof that are functionally active (e.g., active to form Mam:Mam-IP complexes), un-complexed Mam and Mam-IP proteins, and derivatives, analogs, and fragments thereof, and nucleic acids encoding the members of a Mam:Mam-IP complex, or functionally active derivatives or fragments thereof (e.g., for use in gene therapy). In a specific embodiment are derivatives, homologs or fragments of Mam and/or a Mam-IP that increase and/or stabilize Mam:Mam-IP complex formation. Examples of other agonists can be identified using in vitro assays or animal models, examples of which are described supra, and in Section 5.10, infra.

5.6.3.1 Promotion of the Complex Function

In specific embodiments, Therapeutics that promote Mam:Mam-IP complex function, or promote Mip30 or Mip6 function, are administered therapeutically (including prophylactically): (1) in diseases or disorders involving an absence or decreased (relative to normal or desired) level of Mam:Mam-IP complex, or a Mip30 or Mip6 protein, for example, in patients where Mam:Mam-IP complexes (or the individual components necessary to form the complexes), or where Mip30 or Mip6 protein is lacking, genetically defective, biologically inactive or underactive, or under-expressed; or (2) in diseases or disorders wherein in vitro (or in vivo) assays (see infra) indicate the utility of Mam:Mam-IP complex, or Mip30 or Mip6 agonist administration. The absence or decreased level of Mam:Mam-IP complex, or Mip30 or Mip6 protein or function, can be readily detected, e.g., by obtaining a patient tissue sample (e.g., from biopsy tissue) and assaying in vitro for RNA protein levels, activity of the expressed Mam:Mam-IP complex (or for the concurrent expression of mRNA encoding the two components of the Mam:Mam-IP complex), or Mip30 or Mip6 RNA, protein or activity. Many methods standard in the art can be thus employed, including but not limited to immunoassays to detect and/or visualize Mam:Mam-IP complexes (or the individual components of Mam:Mam-IP complexes), or Mip30 or Mip6 protein (e.g., Western blot, immunoprecipitation followed by sodium dodecyl sulfate polyacrylamide gel electrophoresis, immunocytochemistry, etc.) and/or hybridization assays to detect expression of the mRNA encoding the individual protein components of the Mam:Mam-IP complexes by detecting and/or visualizing Mam and a Mam-IP mRNA concurrently or separately using, e.g., Northern blot assays, dot blots, in situ hybridization, etc.

5.6.3.2 Increasing Mam or Mam-IP Levels

In a specific embodiment, the activity or level of Mam is increased by administration of a Mam-IP, or derivative or analog thereof, a nucleic acid encoding a Mam-IP, or an antibody that immunospecifically binds a Mam-IP, or a fragment or derivative of the antibody contains the binding domain thereof. In another specific embodiment, the activity or levels of a Mam-IP are increased by administration of Mam, or derivative or analog thereof, a nucleic acid encoding Mam, or an antibody that immunospecifically binds Mam or a fragment or derivative of the antibody contains the binding domain thereof.

5.6.4 Origin of the Therapeutic

Generally, administration of products of a species origin or species reactivity (in the case of antibodies) that is the same species as that of the patient is preferred. Thus, in a preferred embodiment, a human Mam:Mam-IP complex, or Mip30 or Mip6 protein, or derivative or analog thereof, nucleic acids encoding the members of the human Mam:Mam-IP complex, or human Mip30 or human Mip6, or a derivative or analog thereof, or an antibody to a human Mam:Mam-IP complex, or Mip30 or Mip6 protein, or derivative thereof, is therapeutically or prophylactically administered to a human patient.

5.6.5 Determination of the Effect of the Therapeutic

Preferably, suitable in vitro or in vivo assays are utilized to determine the effect of a specific Therapeutic and whether its administration is indicated for treatment of the affected tissue.

In various specific embodiments, in vitro assays can be carried out with representative cells or cell types involved in a patient's disorder to determine if a Therapeutic has a desired effect upon such cell types.

Compounds for use in therapy can be tested in suitable animal model systems prior to testing in humans, including but not limited to rats, mice, chicken, cows, monkeys, rabbits, etc. For in vivo testing, prior to administration to humans, any animal model system known in the art may be used. Additional descriptions and sources of Therapeutics that can be used according to the invention are found in Sections 5.1-5.3 and 5.8 herein.

5.6.6 Neurodegenerative Disorders

Mam and certain binding partners of Mam (Notch) have been implicated in neurodegenerative disease. Within the developing mammalian nervous system, expression patterns of Notch homologs have been shown to be prominent in particular regions of the ventricular zone of the spinal cord, as well as in components of the peripheral nervous system, in an overlapping but non-identical pattern. Notch expression in the nervous system appears to be limited to regions of cellular proliferation, and is absent from nearby populations of recently differentiated cells. A rat Notch ligand is also expressed within the developing spinal cord, in distinct bands of the ventricular zone that overlap with the expression domains of the Notch genes. The spatio-temporal expression pattern of this ligand correlates well with the patterns of cells committing to spinal cord neuronal fates, which demonstrates the usefulness of Notch as a marker of populations of cells for neuronal fates. Accordingly, Therapeutics of the invention, particularly but not limited to those that modulate (or supply) Mam:IP and complexes of Mam and Mam-IPs may be effective in treating or preventing neurodegenerative disease. Therapeutics of the invention that modulate Mam:Mam-IP complexes involved in neurodegenerative disorders can be assayed by any method known in the art for efficacy in treating or preventing such neurodegenerative diseases and disorders. Such assays include in vitro assays for regulated cell secretion, protein trafficking, and/or folding or inhibition of apoptosis or in vivo assays using animal models of neurodegenerative and/or developmental diseases or disorders, or any of the assays described in Sections 5.7.6 infra. Potentially effective Therapeutics, for example but not by way of limitation, promote regulated cell maturation and prevent cell apoptosis in culture, or reduce neurodegeneration in animal models in comparison to controls.

Once a neurodegenerative disease or disorder has been shown to be amenable to treatment by modulation of Mam:Mam-IP complex activity, that neurodegenerative disease or disorder can be treated or prevented by administration of a Therapeutic that modulates Mam:Mam-IP complex formation (including supplying Mam:Mam-IP complexes).

Such diseases include all degenerative disorders involved with aging, especially osteoarthritis and neurodegenerative disorders. Neurodegenerative disorders that can be treated or prevented include but are not limited to those listed in Table I (see Isslebacher et al., 1997, In: Harrison's Principals of Internal Medicine, 13^{th} Ed., McGraw Hill, New York).

TABLE I
NEURODEGENERATIVE DISORDERS

Progressive dementia in the absence of other
neurological signs
Alzheimer's Disease (or early-onset AD)
Senile dementia of the Alzheimer's type (or
late onset AD)
Pick's Disease
Syndromes combining progressive dementia with
prominent neurological abnormalities
Huntington's disease
Multiple system atrophy (dementia combined with ataxia,
Parkinson's disease, etc.)
Progressive supranuclear palsy
Diffuse Lewy body disease
Corticodentatonigral degeneration
Hallervorden-Spatz disease
Progressive familial myoclonic epilepsy
Syndromes of gradually developing abnormalities
of posture and movement
Parkinson's disease
Striatonigral degeneration
Progressive supranuclear palsy
Torsion dystonia
Spasmodic torticollis and other restricted dyskinesias
Familial tremor
Gilles de la Tourette syndrome
Syndromes of progressive ataxia
Cerebellar cortical degeneration
Olivopontocerebellar atrophy
Friedrich's ataxia and related spinocerebellar
degenerations
Shy-Drager syndrome
Subacute necrotizing encephalopathy
Motor neuron disease without sensory changes
Amyotrophic lateral sclerosis
Infantile spinal muscular atrophy
Juvenile spinal muscular atrophy
Other forms of familial spinal muscular atrophy
Primary lateral sclerosis
Hereditary spastic paraplegia
Motor neuron disease with sensory changes
Peroneal muscular atrophy
Hypertrophic interstitial polyneuropathy
Other forms of chronic progressive neuropathy
Syndromes of progressive visual loss
Retinitis pigmentosa

5.6.7 Oncogenesis

5.6.7.1 Malignancies

Components of Mam:Mam-IP complexes (i.e., Mam, Notch and Mip1 protein) have been implicated in regulation of cell proliferation. Accordingly, Therapeutics of the invention may be useful in treating or preventing diseases or disorders associated with cell hyperproliferation or loss of control of cell proliferation, particularly cancers, malignancies and tumors. Therapeutics of the invention can be assayed by any method known in the art for efficacy in treating or preventing malignancies and related disorders. Such assays include in vitro assays using transformed cells or cells derived from the tumor of a patient, or in vivo assays using animal models of cancer or malignancies, or any of the assays described in Sections 5.7 infra. Potentially effective Therapeutics, for example but not by way of limitation, inhibit proliferation of tumors or transformed cells in culture, or cause regression of tumors in animal models in comparison to controls, e.g., as described in Section 5.7, infra.

Accordingly, once a malignancy or cancer has been shown to be amenable to treatment by modulating (i.e., inhibiting, antagonizing, enhancing or agonizing) Mam:Mam-IP complex activity, or modulating Mip30 or Mip6, activity, that cancer or malignancy can be treated or prevented by administration of a Therapeutic that modulates Mam:Mam-IP complex formation and function, or Mip30 or Mip6 function, including supplying Mam:Mam-IP complexes and the individual binding partners of a Mam:Mam-IP complex. Such cancers and malignancies include but are not limited to those listed in Table II (for a review of such disorders, see Fishman et al., 1985, Medicine, 2d Ed., J.B. Lippincott Co., Philadelphia).

TABLE II
MALIGNANCIES AND RELATED DISORDERS

Leukemia

acute leukemia

acute lymphocytic leukemia
acute myelocytic leukemia

myeloblastic-type
promyelocytic-type
myelomonocytic-type
monocytic-type
erythroleukemia

chronic leukemia

chronic myelocytic (granulocytic) leukemia
chronic lymphocytic leukemia

Polycythemia vera
Lymphoma

Hodgkin's disease
non-Hodgkin's disease

Multiple myeloma
Waldenström's macroglobulinemia
Heavy chain disease
Solid tumors

sarcomas and carcinomas

fibrosarcoma
myxosarcoma
liposarcoma
chondrosarcoma
osteogenic sarcoma
chordoma
angiosarcoma
endotheliosarcoma
lymphangiosarcoma
lymphangioendotheliosarcoma
synovioma
mesothelioma
Ewing's tumor
leiomyosarcoma
rhabdomyosarcoma
colon carcinoma
pancreatic cancer
breast cancer
ovarian cancer
prostate cancer
squamous cell carcinoma
basal cell carcinoma
adenocarcinoma
sweat gland carcinoma
sebaceous gland carcinoma
papillary carcinoma
papillary adenocarcinomas
cystadenocarcinoma
medullary carcinoma
bronchogenic carcinoma
renal cell carcinoma
hepatoma
bile duct carcinoma
choriocarcinoma
seminoma
embryonal carcinoma
Wilms' tumor
cervical cancer
uterine cancer
testicular tumor
lung carcinoma
small cell lung carcinoma
bladder carcinoma
epithelial carcinoma
glioma
astrocytoma
medulloblastoma
craniopharyngioma
ependymoma
pinealoma
hemangioblastoma
acoustic neuroma
oligodendroglioma
menangioma
melanoma
neuroblastoma
retinoblastoma

In specific embodiments, malignancy or dysproliferative changes (such as metaplasias and dysplasias), or hyperproliferative disorders, are treated or prevented in the bladder, breast, colon, lung, prostate, pancreas, or uterus.

5.6.7.2 Premalignant Conditions

The Therapeutics of the invention that are effective in treating cancer or malignancies (e.g., as described above) can also be administered to treat premalignant conditions and to prevent progression to a neoplastic or malignant state, including but not limited to those disorders listed in Table II. Such prophylactic or therapeutic use is indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W.B. Saunders Co., Philadelphia, pp. 68-79). Hyperplasia is a form of controlled cell proliferation involving an increase in cell number in a tissue or organ, without significant alteration in structure or function. As but one example, endometrial hyperplasia often precedes endometrial cancer. Metaplasia is a form of controlled cell growth in which one type of adult cell or fully differentiated cell substitutes for another type of adult cell. Metaplasia can occur in epithelial or connective tissue cells. A typical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, skin, oral cavity, and gall bladder.

Alternatively or in addition to the presence of abnormal cell growth characterized as hyperplasia, metaplasia, or dysplasia, the presence of one or more characteristics of a transformed phenotype, or of a malignant phenotype, displayed in vivo or displayed in vitro by a cell sample from a patient, can indicate the desirability of prophylactic/therapeutic administration of a Therapeutic of the invention that modulates Mam:Mam-IP complex activity, or that modulates Mip30 or Mip6 activity. As mentioned supra, such characteristics of a transformed phenotype include morphological changes, looser substratum attachment, loss of contact inhibition, loss of anchorage dependence, protease release, increased sugar transport, decreased serum requirement, expression of fetal antigens, disappearance of the 250,000 dalton cell surface protein, etc. (see also Id., pp. 84-90 for characteristics associated with a transformed or malignant phenotype).

In a specific embodiment, leukoplakia, a benign-appearing hyperplastic or dysplastic lesion of the epithelium, or Bowen's disease, a carcinoma in situ, are pre-neoplastic lesions indicative of the desirability of prophylactic intervention.

In another embodiment, fibrocystic disease (cystic hyperplasia, mammary dysplasia, particularly adenosis (benign epithelial hyperplasia)) is indicative of the desirability of prophylactic intervention.

In other embodiments, a patient that exhibits one or more of the following predisposing factors for malignancy is treated by administration of an effective amount of a Therapeutic: a chromosomal translocation associated with a malignancy (e.g., the Philadelphia chromosome for chronic myelogenous leukemia, t(14; 18) for follicular lymphoma, etc.), familial polyposis or Gardner's syndrome (possible forerunners of colon cancer), benign monoclonal gammopathy (a possible forerunner of multiple myeloma), and a first degree kinship with persons having a cancer or precancerous disease showing a Mendelian (genetic) inheritance pattern (e.g., familial polyposis of the colon, Gardner's syndrome, hereditary exostosis, polyendocrine adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome; see Robbins and Angell, 1976, Basic Pathology, 2nd Ed., W.B. Saunders Co., Philadelphia, pp. 112-113, etc.)

In another specific embodiment, a Therapeutic of the invention is administered to a human patient to prevent progression to breast, colon, lung, pancreatic, prostate or uterine cancer, or melanoma or sarcoma.

5.6.7.3 Hyperproliferative and Dysproliferative Disorders

In another embodiment of the invention, a Therapeutic is administered to treat or prevent hyperproliferative or benign dysproliferative disorders. Therapeutics of the invention can be assayed by any method known in the art for efficacy in treating or preventing hyperproliferative diseases or disorders, such assays include in vitro cell proliferation assays, in vitro or in vivo assays using animal models of hyperproliferative diseases or disorders, or any of the assays described in Section 5.7, infra. Potentially effective Therapeutics include but are not limited to, Therapeutics that reduce cell proliferation in culture or inhibit growth or cell proliferation in animal models in comparison to controls.

Accordingly, once a hyperproliferative disorder has been shown to be amenable to treatment by modulation of Mam:Mam-IP complex activity, or by modulation of Mip30 or Mip6 protein activity, that hyperproliferative disease or disorder can be treated or prevented by administration of a Therapeutic that modulates Mam:Mam-IP complex formation, or that modulates Mip30 or Mip6 activity (including supplying a Mam:Mam-IP complex and/or the individual binding partners of a Mam:Mam-IP complex).

Specific embodiments are directed to treatment or prevention of cirrhosis of the liver (a condition in which scarring has overtaken normal liver regeneration processes), treatment of keloid (hypertrophic scar) formation (disfiguring of the skin in which the scarring process interferes with normal renewal), psoriasis (a common skin condition characterized by excessive proliferation of the skin and delay in proper cell fate determination), benign tumors, fibrocystic conditions, and tissue hypertrophy (e.g., prostatic hyperplasia).

5.6.8 Gene Therapy

In a specific embodiment, a nucleic acid molecule comprising a sequence encoding Mam and/or a Mam-IP, or a Mip30 or Mip6 protein, or a functional derivative thereof, are administered to modulate Mam:Mam-IP complexes, or to modulate Mip30 or Mip6 function, by way of gene therapy. In more specific embodiments, a nucleic acid or nucleic acids encoding both Mam and a Mam-IP (e.g., Mip1, Mip30, Mip6), or functional derivatives thereof, are administered by way of gene therapy. Gene therapy refers to therapy performed by the administration of a nucleic acid molecule to a subject. In this embodiment of the invention, the nucleic acid molecule produces its encoded protein(s) that mediates a therapeutic effect by modulating the Mam:Mam-IP complex, or by modulating Mip30 or Mip6 function.

Any of the methods for gene therapy available in the art can be used according to the present invention. Exemplary methods are described below.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; Morgan and Anderson, 1993, Ann. Rev. Biochem. 62:191-217; and May, 1993, TIBTECH 11:155-215). Methods commonly known in the art for recombinant DNA technology which can be used are described in Ausubel et al. (eds.), 1993, Current Protocols in Molecular Biology, John Wiley & Sons, NY) and Kriegler, 1990, Gene Transfer and Expression, A Laboratory Manual, Stockton Press, NY.

In a preferred aspect, the Therapeutic comprises a Mam and/or a Mam-IP nucleic acid, or a Mip30 or Mip6 nucleic acid, that is part of an expression vector that expresses the Mam or Mam-IP protein(s), or expresses a Mip30 or Mip6 protein, or fragment or a chimeric protein thereof, in a suitable host. In particular, such a nucleic acid has a promoter(s) operably linked to the Mam and/or the Mam-IP coding region(s), or linked to the Mip30 or Mip6 coding region, said promoter(s) being inducible or constitutive, and optionally, tissue-specific. In another particular embodiment, a nucleic acid molecule is used in which the Mam and/or Mam-IP coding sequence, or the Mip30 or Mip6 coding sequences, and any other desired sequences, are flanked by regions that promote homologous recombination at a desired site in the genome, thus providing for intra-chromosomal expression of the Mam and the Mam-IP nucleic acids (Koller and Smithies, 1989, Proc. Natl. Acad. Sci. USA 86:8932-8935, Zijlstra et al., 1989, Nature 342:435-438).

Delivery of the nucleic acid into a patient may be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo and ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see, U.S. Pat. No. 4,980,286), or by direct injection of naked DNA, or by use of microparticle bombardment (e.g., a gene gun; Biolistic, Dupont), or coating with lipids or cell-surface receptors or transfecting agents, or by encapsulation in liposomes, microparticles, or microcapsules, or by administering it in linkage to a peptide which is known to enter the nucleus, or by administering it in linkage to a ligand subject to receptor-mediated endocytosis (see, e.g., Wu and Wu, 1987, J. Biol. Chem. 262:4429-4432), which can be used to target cell types specifically expressing the receptors, etc. In another embodiment, a nucleic acid-ligand complex can be formed in which the ligand comprises a fusogenic viral peptide that disrupts endosomes, preventing lysosomal degradation of the nucleic acid. In yet another embodiment, the nucleic acid can be targeted in vivo for cell specific uptake and expression by targeting a specific receptor (see, e.g., International Patent Publications WO 92/06180 by Wu et al., WO 92/22635 by Wilson et al., WO 92/20316 by Findeis et al., WO 93/14188 by Clarke et al., and WO 93/20221 by Young). Alternatively, the nucleic acid can be introduced intracellularly and incorporated within host cell DNA for expression, by homologous recombination (Koller and Smithies, 1989, Proc. Natl. Acad. Sci. USA 86:8932-8935, Zijlstra et al., 1989, Nature 342:435-438).

In a specific embodiment, a viral vector that contains the Mam and/or the Mam-IP encoding nucleic acid sequence, or the Mip30 or Mip6 encoding nucleic acid sequence, is used. For example, a retroviral vector can be used (see Miller et al., 1993, Meth. Enzymol. 217:581-599). These retroviral vectors have been modified to delete retroviral sequences that are not necessary for packaging of the viral genome and integration into host cell DNA. The Mam and/or Mam-IP preferably both Mam and Mam-IP) encoding nucleic acids, or Mip30 or Mip6 encoding nucleic acids, to be used in gene therapy is/are cloned into the vector, which facilitates delivery of the gene into a patient. More detail about retroviral vectors can be found in Boesen et al., 1994, Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy include: Clowes et al., 1994, J. Clin. Invest. 93:644-651, Kiem et al., 1994, Blood 83:1467-1473, Salmons and Gunzberg, 1993, Human Gene Therapy 4:129-141, and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10, demonstrated the use of adenovirus vectors to transfer genes to the respiratory epithelia of rhesus monkeys. Other instances of the use of adenoviruses in gene therapy can be found in Rosenfeld et al., 1991, Science 252:431-434, Rosenfeld et al., 1992, Cell 68:143-155, and Mastrangeli et al., 1993, J. Clin. Invest. 91:225-234.

Adeno-associated virus (AAV) has also been proposed for use in gene therapy (Walsh et al., 1993, Proc. Soc. Exp. Biol. Med. 204:289-300).

Another approach to gene therapy involves transferring a gene into cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, 1993, Meth. Enzymol. 217:599-618, Cohen et al., 1993, Meth. Enzymol. 217:618-644, Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell, and is heritable and expressible by its cell progeny.

The resulting recombinant cells can be delivered to a patient by various methods known in the art. In a preferred embodiment, epithelial cells are injected, e.g., subcutaneously. In another embodiment, recombinant skin cells may be applied as a skin graft onto the patient. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes, and blood cells, such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, a Mam and/or Mam-IP (preferably both Mam and Mam-IP) encoding nucleic acid molecule, or a Mip30 or Mip6 encoding nucleic acid molecule, is/are introduced into the cells such that the gene or genes are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention. Such stem cells include but are not limited to hematopoietic stem cells (HSC), stem cells of epithelial tissues such as the skin and the lining of the gut, embryonic heart muscle cells, liver stem cells (International Patent Publication WO 94/08598), and neural stem cells (Stemple and Anderson, 1992, Cell 71:973-985).

Epithelial stem cells (ESCs) or keratinocytes can be obtained from tissues such as the skin and the lining of the gut by known procedures (Rheinwald, 1980, Meth. Cell Bio. 21:229). In stratified epithelial tissue such as the skin, renewal occurs by mitosis of stem cells within the germinal layer, the layer closest to the basal laming Stem cells within the lining of the gut provide for a rapid renewal rate of this tissue. ESCs or keratinocytes obtained from the skin or lining of the gut of a patient or donor can be grown in tissue culture (Rheinwald, 1980, Meth. Cell Bio. 21α:229; Pittelkow and Scott, 1986, Mayo Clinic Proc. 61:771). If the ESCs are provided by a donor, a method for suppression of host versus graft reactivity (e.g., irradiation, drug or antibody administration to promote moderate immunosuppression) can also be used.

With respect to hematopoietic stem cells (HSC), any technique which provides for the isolation, propagation, and maintenance in vitro of HSCs can be used in this embodiment of the invention. Techniques by which this may be accomplished include (a) the isolation and establishment of HSC cultures from bone marrow cells isolated from the future host, or a donor, or (b) the use of previously established long-term HSC cultures, which may be allogeneic or xenogeneic. Non-autologous HSC are used preferably in conjunction with a method of suppressing transplantation immune reactions of the future host/patient. In a particular embodiment of the present invention, human bone marrow cells can be obtained from the posterior iliac crest by needle aspiration (see, e.g., Kodo et al., 1984, J. Clin. Invest. 73:1377-1384). In a preferred embodiment of the present invention, the HSCs can be made highly enriched or in substantially pure form. This enrichment can be accomplished before, during, or after long-term culturing, and can be done by any technique known in the art. Long-term cultures of bone marrow cells can be established and maintained by using, for example, modified Dexter cell culture techniques (Dexter et al., 1977, J. Cell Physiol. 91:335) or Witlock-Witte culture techniques (Witlock and Witte, 1982, Proc. Natl. Acad. Sci. USA 79:3608-3612).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

Additional methods can be adapted for use to deliver a nucleic acid molecule encoding the Mam and/or Mam-IP proteins, or functional derivatives thereof, e.g., as described in Sections 5.1 and 5.2, supra.

5.6.9 Use of Antisense Oligonucleotides for Suppression of Mam:Mam-IP Complexes or for Suppression of Mip30 or Mip6 Protein Expression

In a specific embodiment, Mam:Mam-IP complex function or Mip30 or Mip6 protein function is inhibited by use of antisense nucleic acids for Mam and/or a Mam-IP, (preferably both Mam and the Mam-IP), or individual antisense nucleic acids for Mip30 or Mip6. The present invention provides the therapeutic or prophylactic use of nucleic acids of at least six nucleotides that are antisense to a gene or cDNA encoding Mam and/or a Mam-IP, or encoding Mip30 or Mip6, or a portion thereof. A Mam or a Mam-IP “antisense” nucleic acid as used herein refers to a nucleic acid capable of hybridizing to a portion of Mam or a Mam-IP nucleic acid (preferably mRNA) by virtue of some sequence complementarity. The antisense nucleic acid may be complementary to a coding and/or noncoding region of a Mam or Mam-IP mRNA. Such antisense nucleic acids have utility as Therapeutics that inhibit Mam:Mam-IP complex formation or activity, or Mip30 or Mip6 protein function or activity, and can be used in the treatment or prevention of disorders as described, supra.

The antisense nucleic acids of the invention can be oligonucleotides that are double-stranded or single-stranded, RNA or DNA or a modification or derivative thereof, which can be directly administered to a cell, or which can be produced intracellularly by transcription of exogenous, introduced sequences.

In another embodiment, the invention is directed to methods for inhibiting the expression of Mam and/or a Mam-IP nucleotide sequence, or individual Mip30 or Mip6 nucleotide sequences, in a prokaryotic or eukaryotic cell comprising providing the cell with an effective amount of a composition comprising an antisense nucleic acid of Mam and Mam-IP, or an antisense nucleic acid of Mip30 or Mip6, or a derivative thereof, of the invention.

The Mam and/or Mam-IP antisense nucleic acids are of at least six nucleotides and are preferably oligonucleotides (ranging from 6 to about 200 oligonucleotides). In specific aspects, the oligonucleotide is at least about 10 nucleotides, at least about 15 nucleotides, at least about 100 nucleotides, or at least about 200 nucleotides. The oligonucleotides can be DNA or RNA or chimeric mixtures or derivatives or modified versions thereof, single-stranded or double-stranded. The oligonucleotide can be modified at the base moiety, sugar moiety, or phosphate backbone. The oligonucleotide may include other appending groups such as peptides, or agents facilitating transport across the cell membrane (see, e.g., Letsinger et al., 1989, Proc. Natl. Acad. Sci. U.S.A. 86:6553-6556, Lemaitre et al., 1987, Proc. Natl. Acad. Sci. 84:648-652, PCT Publication No. WO 88/09810, published Dec. 15, 1988) transport across the blood-brain barrier (see, e.g., PCT Publication No. WO 89/10134, published Apr. 25, 1988), hybridization-triggered cleavage agents (see, e.g., Krol et al., 1988, BioTechniques 6:958-976), or intercalation with other agents (see, e.g., Zon, 1988, Pharm. Res. 5:539-549).

In a preferred aspect of the invention, a Mam and/or Mam-IP antisense oligonucleotide is provided, preferably as single-stranded DNA. The oligonucleotide may be modified at any position on its structure with constituents generally known in the art.

The Mam and/or Mam-IP antisense oligonucleotides may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5N-methoxycarboxymethyluracil, S-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

In another embodiment, the oligonucleotide comprises at least one modified sugar moiety selected from the group including but not limited to arabinose, 2-fluoroarabinose, xylulose, and hexose.

In yet another embodiment, the oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, or a formacetal or analog thereof. In yet another embodiment, the oligonucleotide is a 2-anomeric oligonucleotide. An anomeric oligonucleotide forms specific double-stranded hybrids with complementary RNA in which, contrary to the usual β-units, the strands run parallel to each other (Gautier et al., 1987, Nucl. Acids Res. 15:6625-6641).

The oligonucleotide may be conjugated to another molecule, e.g., a peptide, hybridization triggered cross-linking agent, transport agent, hybridization-triggered cleavage agent, etc.

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al., 1988, Nucl. Acids Res. 16:3209, methylphosphonate oligonucleotides can be prepared by use of controlled pore glass polymer supports (Sarin et al., 1988, Proc. Natl. Acad. Sci. U.S.A. 85:7448-7451), etc.

In a specific embodiment, the Mam and/or Mam-IP antisense oligonucleotides comprise catalytic RNAs, or ribozymes (see, e.g., PCT International Publication WO 90/11364, published Oct. 4, 1990, Sarver et al., 1990, Science 247:1222-1225). In another embodiment, the oligonucleotide is a 2-0-methylribonucleotide (Inoue et al., 1987, Nucl. Acids Res. 15:6131-6148), or a chimeric RNA-DNA analog (Inoue et al., 1987, FEBS Lett. 215:327-330).

In an alternative embodiment, the Mam and/or Mam-IP antisense nucleic acids of the invention are produced intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding Mam and/or a Mam-IP (preferably, both a Mam and a Mam-IP antisense nucleic acid) antisense nucleic acid(s), or individual Mip30 or Mip6 antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art to be capable of replication and expression in mammalian cells. Expression of the sequences encoding the Mam and/or Mam-IP antisense RNAs can be by any promoter known in the art to act in mammalian, preferably human, cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), etc.

The antisense nucleic acids of the invention comprise a sequence complementary to at least a portion of an RNA transcript of a Mam or a Mam-IP gene, preferably a human Mam or Mam-IP gene. However, absolute complementarity, although preferred, is not required. A sequence “complementary to at least a portion of an RNA,” as referred to herein, means a sequence having sufficient complementarity to be able to hybridize with the RNA, forming a stable duplex; in the case of double-stranded Mam or Mam-IP antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a Mam or Mam-IP RNA it may contain and still form a stable duplex (or triplex, as the case may be). One skilled in the art can ascertain a tolerable degree of mismatch by use of standard procedures to determine the melting point of the hybridized complex.

The Mam or Mam-IP antisense nucleic acid can be used to treat (or prevent) disorders of a cell type that expresses, or preferably overexpresses, the Mam:Mam-IP complex, or the Mip30 or Mip6 protein. In a preferred embodiment, single-stranded DNA antisense Mam and Mam-IP oligonucleotides, or single-stranded DNA antisense to the same, or individual Mip30 or Mip6 antisense oligonucleotides, or single-stranded DNA antisense to the same, is used.

Cell types that express or overexpress Mam and/or Mam-IP mRNA, or Mip30 or Mip6 RNA can be identified by various methods known in the art. Such methods include, but are not limited to, hybridization with Mam- or Mam-IP-specific nucleic acids (e.g., by Northern blot hybridization, dot blot hybridization, in situ hybridization), or by observing the ability of RNA from the cell type to be translated in vitro into Mam or the Mam-IP, e.g., by immunohistochemistry, ELISA, etc. In a preferred aspect, primary tissue from a patient can be assayed for Mam and/or Mam-IP expression prior to treatment, e.g., by immunocytochemistry or in situ hybridization.

Pharmaceutical compositions of the invention (see Section 5.8, infra), comprising an effective amount of a Mam and/or a Mam-IP antisense nucleic acid in a pharmaceutically acceptable carrier, can be administered to a patient having a disease or disorder that is of a type that expresses or overexpresses Mam:Mam-IP complexes, Mam and/or Mam-IP mRNA, or Mip30 or Mip6 mRNA or protein.

The amount of Mam and/or Mam-IP antisense nucleic acid that will be effective in the treatment of a particular disorder or condition will depend on the nature of the disorder or condition, and can be determined by standard clinical techniques. Where possible, it is desirable to determine the antisense cytotoxicity in vitro, and then in useful animal model systems prior to testing and use in humans.

In a specific embodiment, pharmaceutical compositions comprising Mam or Mam-IP antisense nucleic acids are administered via liposomes, microparticles, or microcapsules. In various embodiments of the invention, it may be useful to use such compositions to achieve sustained release of the Mam and/or Mam-IP antisense nucleic acids. In a specific embodiment, it may be desirable to utilize liposomes targeted via antibodies to specific identifiable central nervous system cell types (Leonetti et al., 1990, Proc. Natl. Acad. Sci. U.S.A. 87:2448-2451, Renneisen et al., 1990, J. Biol. Chem. 265:16337-16342).

5.7 Assays of Mam:Mam-IP Complexes, and Mip30 or Mip6 Proteins

The functional activity of a Mam:Mam-IP complex, or the functional activity of a Mip30 or Mip6 protein, and derivatives, fragments and analogs thereof, can be assayed by various methods known in the art. Potential modulators (e.g., inhibitors, agonists and antagonists) of Mam:Mam-IP complex activity, or of Mip30 or Mip6 activity (e.g., anti-Mam:Mam-IP, anti-Mip30 or anti-Mip6 antibodies, and Mam or Mam-IP antisense nucleic acids) can be assayed for the ability to modulate Mam:Mam-IP complex formation and/or activity, and for the ability to modulate Mip30 or Mip6 activity.

5.7.1 Immunoassays

For example, in one embodiment, where one is assaying for the ability to bind or compete with wild-type Mam:Mam-IP complexes, or Mip30 or Mip6 protein, for binding to anti-Mam:Mam-IP antibodies, or anti-Mip30 or anti-Mip6 antibodies, various immunoassays known in the art can be used, including but not limited to competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), Western blots, precipitation reactions, agglutination assays (e.g., gel agglutination assays, hemagglutination assays), complement fixation assays, immunofluorescence assays, protein A assays, immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

5.7.2 Assays for Gene Expression

The expression of the Mam and/or Mam-IP genes (both endogenous genes and those expressed from cloned DNA containing these genes) can be detected using techniques known in the art, including but not limited to Southern hybridization (Southern, 1975, J. Mol. Biol. 98: 503-517), Northern hybridization (e.g., Freeman et al., 1983, Proc. Natl. Acad. Sci. USA 80: 4094-4098), restriction endonuclease mapping (Sambrook et al., 1989, Molecular Cloning, A Laboratory Manual, 2^{nd} Ed., Cold Spring Harbor Laboratory Press, New York), DNA sequence analysis, polymerase chain reaction amplification (PCR, U.S. Pat. Nos. 4,683,202, 4,683,195, and 4,889,818; Gyllenstein et al., 1988, Proc. Natl. Acad. Sci. USA 85:7652-7657; Ochman et al., 1988, Genetics 120:621-623; and Loh et al., 1989, Science 243:217-220), or RNase protection (Current Protocols in Molecular Biology, John Wiley and Sons, New York, 1997) with probes specific for Mam or Mam-IP genes, in various cell types. Methods of amplification other than PCR commonly known in the art can be employed. In one embodiment, Southern hybridization can be used to detect genetic linkage of Mam or Mam-IP gene mutations to physiological or pathological states. Various cell types, at various stages of development, can be characterized for their expression of Mam and/or a Mam-IP (particularly expression of Mam and/or a Mam-IP at the same time and in the same cells), or Mip30 or Mip6 protein expression. The stringency of the hybridization conditions for northern or Southern blot analysis can be manipulated to ensure detection of nucleic acids with the desired degree of relatedness to the specific probes used. Modifications to these methods and other methods commonly known in the art can be used.

5.7.3 Binding Assays

Derivatives (e.g., fragments) and analogs of Mam-IPs can be assayed for binding to Mam by any method known in the art, for example the modified yeast two hybrid assay system described in Section 6, infra, immunoprecipitation with an antibody that binds to Mam in a complex followed by analysis by size fractionation of the immunoprecipitated proteins (e.g., by denaturing or nondenaturing polyacrylamide gel electrophoresis), Western analysis, non-denaturing gel electrophoresis, etc.

5.7.4 Assays for Biological Activity

One embodiment of the invention provides a method for screening a derivative or analog of Mam for biological activity comprising contacting said derivative or analog of Mam with a protein selected from the group consisting of Mip1, Mip30 and Mip6, and detecting the formation of a complex between said derivative or analog of Mam and said protein; wherein detecting formation of said complex indicates that said derivative or analog of Mam has biological (e.g., binding) activity. Additionally, another embodiment of the invention relates to a method for screening a derivative or analog of a protein selected from the group consisting of Mip1, Mip30 and Mip6 for biological activity comprising contacting said derivative or analog of said protein with Mam; and detecting the formation of a complex between said derivative or analog of said protein and Mam; wherein detecting the formation of said complex indicates that said derivative or analog of said protein has biological activity.

5.7.5 Methods of Modulating the Protein Activity

The present invention also provides methods of modulating the activity of a protein that can participate in a Mam:Mam-IP complex (e.g., Mam, Mip1, Mip30, or Mip6) by administration of a binding partner of that protein, or derivative or analog thereof. Mam and derivatives and analogs thereof, can be assayed for the ability to modulate the activity or level of a Mam-IP by contacting a cell, or administering to an animal, expressing a Mam-IP gene with a Mam protein, or a nucleic acid encoding a Mam protein, or an antibody that immunospecifically binds the Mam protein, or a fragment or derivative of said antibody containing the binding domain thereof, and measuring a change in Mam-IP levels or activity, wherein a change in Mam-IP levels or activity indicates that Mam can modulate Mam-IP levels or activity. Alternatively, a Mam-IP can be assayed for the ability to modulate the activity or levels of a Mam protein by contacting a cell, or administering to an animal, expressing a Mam gene with a Mam-IP, or a nucleic acid encoding a Mam-IP, or an antibody that immunospecifically binds to a Mam-IP, or a fragment or derivative of said antibody containing the binding domain thereof, wherein a change in Mam levels or activity indicates that the Mam-IP can modulate Mam levels or activity.

The Mam:Mam-IP complex, or Mip30 or Mip6 protein, or derivative, analog, or fragment thereof, can also be screened for activity in modulating the activity of Mam and the Mam binding partners particularly Mip1, Mip30 and Mip6 (i.e., the Mam-IPs, involved in particular Mam:Mam-IP complexes). The complexes and proteins of the invention can be screened for the ability to modulate (i.e., increase or decrease) Mam:Mam-IP complexes, as specified below.

Mip30 contains seven C2H2 zinc-finger repeat domains, a HMG-1 and HMG-Y DNA-binding domain (A+T-hook), and a bipartite nuclear localization signal. Mip6 contains a bipartite nuclear localization signal.

5.7.6 Assays for Treatment of Neurodegeneration Disorders

The Mam:Mam-IP complexes particularly the Mam:Mip1, Mam:Mip30 and Mam:Mip6 complexes), derivatives, analogs and fragments thereof, nucleic acids encoding the Mam and Mam-IP genes, anti-Mam:Mam-IP antibodies, and other modulators of Mam:Mam-IP complex activity, can be tested for activity in treating or preventing neurodegenerative disease in in vitro and in vivo assays.

In one embodiment, a Therapeutic of the invention can be assayed for activity in treating or preventing neurodegenerative disease by contacting cultured cells that exhibit an indicator of a neurodegenerative disease, such as overexpression of the β-A4 peptide, in vitro with the Therapeutic, and comparing the level of said indicator in the cells contacted with the Therapeutic with said level of said indicator in cells not so contacted, wherein a lower level in said contacted cells indicates that the Therapeutic has activity in treating or preventing neurodegenerative disease. Specific examples of cell culture models for neurodegenerative disease include, but are not limited to, cultured rat endothelial cells from affected and nonaffected individuals (Maneiro et al., 1997, Methods Find. Exp. Clin. Pharmacol. 19:5-12), P19 murine embryonal carcinoma cells (Hung et al., 1992, Proc. Natl. Acad. Sci. USA 89:9439-9443), and dissociated cell cultures of cholinergic neurons from the nucleus basalis of Meynert (Nakajima et al., 1985, Proc. Natl. Acad. Sci. USA, 82:6325-6329).

In another embodiment, a Therapeutic of the invention can be assayed for activity in treating or preventing neurodegenerative disease by administering the Therapeutic to a test animal that exhibits symptoms of a neurodegenerative disease, such as premature development of cognitive deficiencies in transgenic animals expressing β-APP, or that is predisposed to develop symptoms of a neurodegenerative disease; and measuring the change in said symptoms of the neurodegenerative disease after administration of said Therapeutic, wherein a reduction in the severity of the symptoms of the neurodegenerative disease or prevention of the symptoms of the neurodegenerative disease, indicates that the Therapeutic has activity in treating or preventing neurodegenerative disease. Such a test animal can be any one of a number of animal models known in the art for neurodegenerative disease. These models, including those for Alzheimer's Disease and mental retardation of trisomy 21, which accurately mimic the natural human neurodegenerative disease (Campbell, et al., 1997, Mol. Psychiatry 2:125-129; Schultz et al., 1997, Mol. Cell. Biochem. 174:193-197; Oron et al., 1997, J. Neural. Transm. Suppl. 49:77-84). Examples of specific models, include but are not limited to, the partial trisomy 16 mouse (Holtzman et al., 1996, Proc. Natl. Acad. Sci. USA 93:13333-13338), bilateral nucleus basalis magnocellularis-lesioned rats (Popovic et al., 1996, Int. J. Neurosci. 86:281-299), the aged rat (Muir, 1997, Pharmacol. Biochem. Behav. 56:687-696), the PDAPP transgenic mouse model of Alzheimer's disease (Johnson-Wood et al., 1997, Proc. Natl. Acad. Sci. USA 94:1550-1555), and experimental autoimmune dementia (Oron et al., 1997, J. Neural Transm. Suppl. 49:77-84).

5.7.7 Assays for Treatment of Tumorigenesis

Mam and several of the identified binding partners of Mam (e.g., Mip1) have roles in the control of mitosis and cell proliferation and, therefore, cell-transformation and tumorigenesis. Accordingly, methods of the invention are provided for screening Mam:Mam-IP complexes, proteins, and fragments, derivatives and analogs of the foregoing, for activity in altering cell proliferation, cell transformation and/or tumorigenesis in vitro and in vivo.

The Mam:Mam-IP complexes or Mip30 or Mip6 proteins, derivatives, fragments, and analogs thereof, can be assayed for activity to alter (i.e., increase or decrease) cell proliferation in cultured cells in vitro using methods which are well known in the art for measuring cell proliferation. Specific examples of cell culture models include, but are not limited to, for lung cancer, primary rat lung tumor cells (Swafford et al., 1997, Mol. Cell. Biol., 17:1366-1374) and large-cell undifferentiated cancer cell lines (Mabry et al., 1991, Cancer Cells, 3:53-58), colorectal cell lines for colon cancer (Park and Gazdar, 1996, J. Cell Biochem. Suppl. 24:131-141), multiple established cell lines for breast cancer (Hambly et al., 1997, Breast Cancer Res. Treat. 43:247-258; Gierthy et al., 1997, Chemosphere 34:1495-1505; Prasad and Church, 1997, Biochem. Biophys. Res. Commun. 232:14-19), a number of well-characterized cell models for prostate cancer (Webber et al., 1996, Prostate, Part 1, 29:386-394; Part 2, 30:58-64; and Part 3, 30:136-142; Boulikas, 1997, Anticancer Res. 17:1471-1505), for genitourinary cancers, continuous human bladder cancer cell lines (Ribeiro et al., 1997, Int. J. Radiat. Biol. 72:11-20), organ cultures of transitional cell carcinomas (Booth et al., 1997, Lab Invest. 76:843-857), and rat progression models (Vet et al., 1997, Biochim. Biophys Acta 1360:39-44), and established cell lines for leukemias and lymphomas (Drexler, 1994, Leuk. Res. 18:919-927, Tohyama, 1997, Int. J. Hematol. 65:309-317).

For example, but not by way of limitation, cell proliferation can be assayed by measuring ³H-thymidine incorporation, by direct cell count, by detecting changes in transcriptional activity of known genes, such as proto-oncogenes (e.g., c-fos and c-myc), by detecting changes in cell cycle markers, etc. Accordingly, one embodiment of the present invention provides a method of screening Mam:Mam-IP complexes, or Mip30 or Mip6 protein, and fragments, derivatives, and analogs thereof, for activity in altering (i.e., increasing or decreasing) proliferation of cells in vitro, comprising contacting the cells with a Mam:Mam-IP complex, or a Mip30 or Mip6 protein, or a derivative, analog, or fragment thereof, measuring the proliferation of cells that have been so contacted, and comparing the proliferation of the cells so contacted with a complex or protein of the invention with the proliferation of cells not so contacted with the complex or protein of the invention, wherein in a change in the level of proliferation in said contacted cells indicates that the complex or protein of the invention has activity to alter cell proliferation.

The Mam:Mam-IP complexes, or Mip30 or Mip6 protein, derivative, fragment or analog thereof, can also be screened for activity in inducing or inhibiting cell transformation (or progression to malignant phenotype) in vitro. The complexes and proteins of the invention can be screened by contacting either cells with a normal phenotype (for assaying for cell transformation) or a transformed cell phenotype (for assaying for inhibition of cell transformation) with the complex or protein of the invention, and examining the cells for acquisition or loss of characteristics associated with a transformed phenotype (a set of in vitro characteristics associated with a tumorigenic ability in vivo), for example, but not limited to, colony formation in soft agar, a more rounded cell morphology, looser substratum attachment, loss of contact inhibition, loss of anchorage dependence, release of proteases such as plasminogen activator, increased sugar transport, decreased serum requirement, expression of fetal antigens, disappearance of the 250 kD surface protein, etc. (see Luria et al., 1978, General Virology, 3d Ed., John Wiley & Sons, New York, pp. 436-446).

The Mam:Mam-IP complexes, or Mip30 or Mip6 protein, derivative, fragment, or analog thereof, can also be screened for activity to promote or inhibit tumor formation in vivo in a non-human test animal. A vast number of animal models of hyperproliferative disorders, including tumorigenesis and metastatic spread, are known in the art (see Table 317-1, Chapter 317, “Principals of Neoplasia,” in Harrison's Principals of Internal Medicine, 13th Edition, Isselbacher et al., eds., McGraw-Hill, New York, p. 1814, and Lovejoy et al., 1997, J. Pathol. 181:130-135). Specific examples include for lung cancer, transplantation of tumor nodules into rats (Wang et al., 1997, Ann. Thorac. Surg. 64:216-219) or establishment of lung cancer metastases in SCID mice depleted of NK cells (Yono and Sone, 1997, Gan To Kagaku Ryoho 24:489-494); for colon cancer, colon cancer transplantation of human colon cancer cells into nude mice (Gutman and Fidler, 1995, World J. Surg. 19:226-234), the cotton top tamarin model of human ulcerative colitis (Warren, 1996, Aliment. Pharmacol. Ther. 10 Supp 12:45-47) and mouse models with mutations of the adenomatous polyposis tumor suppressor (Polakis, 1997, Biochim. Biophys. Acta 1332:F127-F147); for breast cancer, transgenic models of breast cancer (Dankort and Muller, 1996, Cancer Treat. Res. 83:71-88; Amundadittir et al., 1996, Breast Cancer Res. Treat. 39:119-135) and chemical induction of tumors in rats (Russo and Russo, 1996, Breast Cancer Res. Treat. 39:7-20); for prostate cancer, chemically-induced and transgenic rodent models, and human xenograft models (Royai et al., 1996, Semin. Oncol. 23:35-40); for genitourinary cancers, induced bladder neoplasm in rats and mice (Oyasu, 1995, Food Chem. Toxicol 33:747-755) and xenografts of human transitional cell carcinomas into nude rats (Jarrett et al., 1995, J. Endourol. 9:1-7); and for hematopoietic cancers, transplanted allogeneic marrow in animals (Appelbaum, 1997, Leukemia 11 (Suppl. 4):S15-S17). Further, general animal models applicable to many types of cancer have been described, including, but not restricted to, the p53-deficient mouse model (Donehower, 1996, Semin. Cancer Biol. 7:269-278), the Min mouse (Shoemaker et al., 1997, Biochem. Biophys. Acta, 1332:F25-F48), and immune responses to tumors in rat (Frey, 1997, Methods, 12:173-188).

For example, the complexes and proteins of the present invention can be administered to non-human test animals (preferably test animals predisposed to develop a type of tumor) and the non-human test animal subsequently examined for an increased incidence of tumor formation in comparison with controls not administered the complex or protein of the invention. Alternatively, the complexes and proteins of the present invention can be administered to non-human test animals having tumors (e.g., animals in which tumors have been induced by introduction of malignant, neoplastic, or transformed cells, or by administration of a carcinogen) and subsequently examining the tumors in the test animals for tumor regression in comparison to controls not administered the complex a protein of the present invention.

In one embodiment of the present invention, a molecule that modulates activity of Mam or a protein selected from the group consisting of Mip1, Mip30 and Mip6, or a complex of Mam and said protein, is identified by contacting one or more candidate molecules with Mam in the presence of said protein; and measuring the amount of complex that forms between Mam and said protein; wherein an increase or decrease in the amount of complex that forms relative to the amount that forms in the absence of the candidate molecules indicates that the molecules modulate the activity of Mam or said protein or said complex of Mam and said protein. In preferred embodiments, modulators are identified by administering a candidate molecule to a transgenic non-human animal expressing both Mam and a Mam-IP from promoters that are not the native Mam or the native Mam-IP promoters, more preferably where the candidate molecule is also recombinantly expressed in the transgenic non-human animal. Alternatively, the method for identifying such modulators can be carried out in vitro, preferably with purified Mam, purified Mam-IP, and a purified candidate molecule.

Methods that can be used to carry out the foregoing are commonly known in the art. Agents to be screened can be provided as mixtures of a limited number of specified compounds, or as compound libraries, peptide libraries and the like. Agents to be screened may also include all forms of antisera, antisense nucleic acids, etc., that can modulate Mam:Mam-IP complex activity, or modulate a Mip30 or Mip6 activity.

Exemplary libraries of candidate molecules are described in Section 5.4.1, supra.

In a specific embodiment, screening can be carried out by contacting the library members with a Mam:Mam-IP complex, or with a Mip30 or Mip6 protein (or encoding nucleic acid molecule or derivative) immobilized on a solid phase, and harvesting those library members that bind to the protein (or nucleic acid or derivative). Examples of such screening methods, termed “panning” techniques, are described by way of example in Parmley and Smith, 1988, Gene 73:305-318; Fowlkes et al., 1992, BioTechniques 13:422-427; International Patent Publication No. WO 94/18318; and in references cited hereinabove.

In a specific embodiment, fragments and/or analogs of Mam or a Mam-IP, especially peptidomimetics, are screened for activity as competitive or non-competitive inhibitors of Mam:Mam-IP complex formation, and thereby inhibit Mam:Mam-IP complex activity.

In a preferred embodiment, molecules that bind to a Mam:Mam-IP complex, or to a Mip30 or Mip6 protein, can be screened for by using the modified yeast two hybrid system described in Section 5.8.1 infra, and exemplified in Section 6.1, infra.

In one embodiment, agents that modulate (i.e., inhibit, antagonize or agonize) Mam:Mam-IP complex activity can be screened for using a binding inhibition assay, wherein agents are screened for their ability to inhibit formation of a Mam:Mam-IP complex under aqueous, or physiological, binding conditions in which Mam:Mam-IP complex formation occurs in the absence of the agent to be tested. Agents that interfere with the formation of Mam:Mam-IP complexes are identified as antagonists of complex formation. Agents that eliminate the formation of Mam:Mam-IP complexes are identified as inhibitors of complex formation. Agents that enhance the formation of Mam:Mam-IP complexes are identified as agonists of complex formation.

Methods for screening may involve labeling the complex proteins with radioligands (e.g., ¹²⁵I, or ³H), magnetic ligands (e.g., paramagnetic beads covalently attached to photobiotin acetate), fluorescent ligands (e.g., fluorescein or rhodamine) or enzyme ligands (e.g., luciferase or beta-galactosidase). The reactants that bind in solution can then be isolated by one of many techniques known in the art, including but not restricted to, co-immunoprecipitation of the labeled moiety using antisera against the unlabeled binding partner (or a binding partner labeled with a distinguishable marker from that used on the labeled moiety), immunoaffinity chromatography, size exclusion chromatography, and gradient density centrifugation. In a preferred embodiment, one binding partner is a small fragment or peptidomimetic that is not retained by a commercially available filter. Upon binding, the labeled species is then unable to pass through the filter, providing for a simple assay of complex formation.

Methods commonly known in the art are used to label at least one of the members of the Mam:Mam-IP complex. Suitable labeling includes, but is not limited to, radiolabeling by incorporation of radiolabeled amino acids, e.g., ³H-leucine or ³⁵S-methionine, radiolabeling by post-translational iodination with ¹²⁵I or ¹³¹I using the chloramine T method, Bolton-Hunter reagents, etc., labeling with ³²P using a kinase and inorganic radiolabeled phosphorous, biotin labeling with photobiotin-acetate and sunlamp exposure, etc. In cases where one of the members of the Mam:Mam-IP complex is immobilized, e.g., as described infra, the free species is labeled. Where neither of the interacting species is immobilized, each can be labeled with a distinguishable marker such that isolation of both moieties can be followed to provide for more accurate quantitation, and to distinguish the formation of homomeric from heteromeric complexes. Methods that utilize accessory proteins that bind to one of the modified interactants to improve the sensitivity of detection, increase the stability of the complex, etc. are provided.

Typical binding conditions are, for example, but not by way of limitation, in an aqueous salt solution of 10-250 mM NaCl, 5-50 mM Tris-HCl, pH 5-8, and 0.5% Triton X-100 or other detergent that improves the specificity of interaction. Metal chelators and/or divalent cations may be added to improve binding and/or reduce proteolysis. Reaction temperatures may include 4, 10, 15, 22, 25, 35, or 42 degrees Celsius, and time of incubation is typically at least 15 seconds, but longer times are preferred to allow binding equilibrium to occur. Particular Mam:Mam-IP complexes can be assayed using routine protein binding assays to determine optimal binding conditions for reproducible binding.

The physical parameters of complex formation can be analyzed by quantitation of complex formation using assay methods specific for the label used, e.g., liquid scintillation spectroscopy for radioactivity detection, enzyme activity measurements for enzyme labeling, etc. The reaction results are then analyzed utilizing Scatchard analysis, Hill analysis, and other methods commonly known in the art (see, e.g., Proteins, Structures, and Molecular Principles, 2^{nd} Edition (1993) Creighton, Ed., W.H. Freeman and Company, New York).

In a second common approach to binding assays, one of the binding species is immobilized on a filter, in a microtiter plate well, in a test tube, to a chromatography matrix, etc., either covalently or non-covalently. Proteins can be covalently immobilized using any method well known in the art, for example, but not limited to the method of Kadonaga and Tjian (1986, Proc. Natl. Acad. Sci. USA 83:5889-5893, 1986), i.e., linkage to a cyanogen-bromide derivatized substrate such as CNBr-Sepahrose 4B. Where needed, the use of spacers can reduce steric hindrance by the substrate. Non-covalent attachment of proteins to a substrate include, but are not limited to, attachment of a protein to a charged surface, binding with specific antibodies, binding to a third unrelated interacting protein.

In one embodiment, immobilized Mam is used to assay for binding with a radioactively-labeled Mam-IP in the presence and absence of a compound to be tested for its ability to modulate Mam:Mam-IP complex formation. The binding partners are allowed to bind under aqueous, or physiological, conditions (e.g., the conditions under which the original interaction was detected). Conversely, in another embodiment, the Mam-IP is immobilized and contacted with the labeled Mam protein or derivative thereof under binding conditions.

Assays of agents (including cell extracts or library pools) for competition for binding of one member of a Mam:Mam-IP complex (or derivatives thereof) with the other member of the Mam:Mam-IP complex (labeled by any means, e.g., those means described supra), are provided to screen for competitors of Mam:Mam-IP complex formation.

In specific embodiments, blocking agents to inhibit non-specific binding of reagents to other protein components, or absorptive losses of reagents to plastics, immobilization matrices, etc., are included in the assay mixture. Blocking agents include, but are not restricted to, bovine serum albumin, beta-casein, nonfat dried milk, Denhardt's reagent, Ficoll, polyvinylpyrolidine, nonionic detergents (NP40, Triton X-100, Tween 20, Tween 80, etc.), ionic detergents (e.g., SDS, LDS, etc.), polyethylene glycol, etc. Appropriate blocking agent concentrations are utilized to allow Mam:Mam-IP complex formation.

After binding is performed, unbound, labeled protein is removed with the supernatant, and the immobilized protein with any bound, labeled protein is washed extensively. The amount of label bound is then quantitated using standard methods known in the art to detect the label.

5.8.1 Assays for Proteins-Protein Interactions

One aspect of the present invention provides methods for assaying and screening fragments, derivatives and analogs of Mam interacting proteins (for binding to a Mam peptide). Derivatives, analogs and fragments of Mam-IPs that interact with Mam can be identified by means of a yeast two hybrid assay system (Fields and Song, 1989, Nature 340:245-246 and U.S. Pat. No. 5,283,173). Because the interactions are screened for in yeast, the intermolecular protein interactions detected in this system occur under physiological conditions that mimic the conditions in mammalian cells (Chien et al., 1991, Proc. Natl. Acad. Sci. USA 88:9578-9581).

Identification of interacting proteins by the improved yeast two hybrid system is based upon the detection of expression of a reporter gene, the transcription of which is dependent upon the reconstitution of a transcriptional regulator by the interaction of two proteins, each fused to one half of the transcriptional regulator. The “bait” (Mam or derivative or analog) and “prey” (proteins to be tested for ability to interact with the bait) proteins are expressed as fusion proteins to a DNA binding domain, and to a transcriptional regulatory domain, respectively, or vice versa. In various specific embodiments, the prey has a complexity of at least about 50, about 100, about 500, about 1,000, about 5,000, about 10,000, or about 50,000; or has a complexity in the range of about 25 to about 100,000, about 100 to about 100,000, about 50,000 to about 100,000, or about 100,000 to about 500,000. For example, the prey population can be one or more nucleic acids encoding mutants of a Mam-IP (e.g., as generated by site-directed mutagenesis or another method of making mutations in a nucleotide sequence). Preferably, the prey populations are proteins encoded by DNA, e.g., cDNA or genomic DNA or synthetically generated DNA. For example, the populations can be expressed from chimeric genes comprising cDNA sequences from an un-characterized sample of a population of cDNA from mammalian RNA.

In a specific embodiment, recombinant biological libraries expressing random peptides can be used as the source of prey nucleic acids.

In another embodiment, the invention provides methods of screening for inhibitors or enhancers of the protein interactants identified herein. Briefly, the protein-protein interaction assay can be carried out as described herein, except that it is done in the presence of one or more candidate molecules. An increase or decrease in reporter gene activity relative to that present when the one or more candidate molecules are absent indicates that the candidate molecule has an effect on the interacting pair. In a preferred method, inhibition of the interaction is selected for (i.e., inhibition of the interaction is necessary for the cells to survive), for example, where the interaction activates the URA3 gene, causing yeast to die in medium containing the chemical 5-fluoroorotic acid (Rothstein, 1983, Meth. Enzymol. 101: 167-180). The identification of inhibitors of such interactions can also be accomplished, for example, but not by way of limitation, using competitive inhibitor assays, as described supra.

In general, proteins of the bait and prey populations are provided as fusion (chimeric) proteins (preferably by recombinant expression of a chimeric coding sequence) comprising each protein contiguous to a pre-selected sequence. For one population, the pre-selected sequence is a DNA binding domain. The DNA binding domain can be any DNA binding domain, as long as it specifically recognizes a DNA sequence within a promoter. For example, the DNA binding domain is of a transcriptional activator or inhibitor. For the other population, the pre-selected sequence is an activator or inhibitor domain of a transcriptional activator or inhibitor, respectively. The regulatory domain alone (not as a fusion to a protein sequence) and the DNA-binding domain alone (not as a fusion to a protein sequence) preferably do not detectably interact (so as to avoid false positives in the assay). The assay system further includes a reporter gene operably linked to a promoter that contains a binding site for the DNA binding domain of the transcriptional activator (or inhibitor). Accordingly, in the present method of the present invention, binding of a Mam fusion protein to a prey fusion protein leads to reconstitution of a transcriptional activator (or inhibitor) which activates (or inhibits) expression of the reporter gene. The activation (or inhibition) of transcription of the reporter gene occurs intracellularly, e.g., in prokaryotic or eukaryotic cells, preferably in cell culture.

The promoter that is operably linked to the reporter gene nucleotide sequence can be a native or non-native promoter of the nucleotide sequence, and the DNA binding site(s) that are recognized by the DNA binding domain portion of the fusion protein can be native to the promoter (if the promoter normally contains such binding site(s)) or non-native to the promoter. Thus, for example, one or more tandem copies (e.g., 4 or 5 copies) of the appropriate DNA binding site can be introduced upstream of the TATA box in the desired promoter (e.g. in the area of about position −100 to about −400). In a preferred aspect, 4 or 5 tandem copies of the 17 bp UAS (GAL4 DNA binding site) are introduced upstream of the TATA box in the desired promoter, which is upstream of the desired coding sequence for a selectable or detectable marker. In a preferred embodiment, the GAL1-10 promoter is operably fused to the desired nucleotide sequence; the GAL1-10 promoter already contains 5 binding sites for GAL4.

Alternatively, the transcriptional activation binding site of the desired gene(s) can be deleted and replaced with GAL4 binding sites (Bartel et al., 1993, BioTechniques 14:920-924, Chasman et al., 1989, Mol. Cell. Biol. 9:4746-4749). The reporter gene preferably contains the sequence encoding a detectable or selectable marker, the expression of which is regulated by the transcriptional activator, such that the marker is either turned on or off in the cell in response to the presence of a specific interaction. Preferably, the assay is carried out in the absence of background levels of the transcriptional activator (e.g., in a cell that is mutant or otherwise lacking in the transcriptional activator). In one embodiment, more than one reporter gene is used to detect transcriptional activation, e.g., one reporter gene encoding a detectable marker and one or more reporter genes encoding different selectable markers. The detectable marker can be any molecule that can give rise to a detectable signal, e.g., a fluorescent protein or a protein that can be readily visualized or that is recognizable by a specific antibody. The selectable marker can be any protein molecule that confers the ability to grow under conditions that do not support the growth of cells not expressing the selectable marker, e.g., the selectable marker is an enzyme that provides an essential nutrient and the cell in which the interaction assay occurs is deficient in the enzyme and the selection medium lacks such nutrient. The reporter gene can either be under the control of the native promoter that naturally contains a binding site for the DNA binding protein, or under the control of a heterologous or synthetic promoter.

The activation domain and DNA binding domain used in the assay can be from a wide variety of transcriptional activator proteins, as long as these transcriptional activators have separable binding and transcriptional activation domains. For example, the GAL4 protein of S. cerevisiae (Ma et al., 1987, Cell 48:847-853), the GCN4 protein of S. cerevisiae (Hope and Struhl, 1986, Cell 46:885-894), the ARD1 protein of S. cerevisiae (Thukral et al., 1989, Mol. Cell. Biol. 9:2360-2369), and the human estrogen receptor (Kumar et al., 1987, Cell 51:941-951), have separable DNA binding and activation domains. The DNA binding domain and activation domain that are employed in the fusion proteins need not be from the same transcriptional activator. In a specific embodiment, a GAL4 or LEXA DNA binding domain is employed. In another specific embodiment, a GAL4 or herpes simplex virus VP16 (Triezenberg et al., 1988, Genes Dev. 2:730-742) activation domain is employed. In a specific embodiment, amino acids 1-147 of GAL4 (Ma et al., 1987, Cell 48:847-853; Ptashne et al., 1990, Nature 346:329-331) is the DNA binding domain, and amino acids 411-455 of VP16 (Triezenberg et al., 1988, Genes Dev. 2:730-742; Cress et al., 1991, Science 251:87-90) comprise the activation domain.

In a preferred embodiment, the yeast transcription factor GAL4 is reconstituted by protein-protein interaction and the host strain is mutant for GAL4. In another embodiment, the DNA-binding domain is Ace1N and/or the activation domain is Ace1, the DNA binding and activation domains of the Ace1 protein, respectively. Ace1 is a yeast protein that activates transcription from the CUP1 operon in the presence of divalent copper. CUP1 encodes metallothionein, which chelates copper, and the expression of CUP1 protein allows growth in the presence of copper, which is otherwise toxic to the host cells. The reporter gene can also be a CUP1-lacZ fusion that expresses the enzyme beta-galactosidase (detectable by routine chromogenic assay) upon binding of a reconstituted Ace1N transcriptional activator (see Chaudhuri et al., 1995, FEBS Letters 357:221-226). In another specific embodiment, the DNA binding domain of the human estrogen receptor is used, with a reporter gene driven by one or three estrogen receptor response elements (Le Douarin et al., 1995, Nucl. Acids. Res. 23:876-878).

The DNA binding domain and the transcriptional activator/inhibitor domain each preferably has a nuclear localization signal (see Ylikomi et al., 1992, EMBO J. 11:3681-3694, Dingwall and Laskey, 1991, TIBS 16:479-481) functional in the cell in which the fusion proteins are to be expressed.

To facilitate isolation of the encoded proteins, the fusion constructs can further contain sequences encoding affinity tags such as glutathione-5-transferase or maltose-binding protein or an epitope of an available antibody, for affinity purification (e.g., binding to glutathione, maltose, or a particular antibody specific for the epitope, respectively) (Allen et al., 1995, TIBS 20:511-516). In another embodiment, the fusion constructs further comprise bacterial promoter sequences for recombinant production of the fusion protein in bacterial cells.

The host cell in which the interaction assay occurs can be any cell, prokaryotic or eukaryotic, in which transcription of the reporter gene can occur and be detected, including, but not limited to, mammalian (e.g., monkey, mouse, rat, human, bovine), chicken, bacterial, or insect cells, and is preferably a yeast cell. Expression constructs encoding and capable of expressing the binding domain fusion proteins, the transcriptional activation domain fusion proteins, and the reporter gene product(s) are provided within the host cell, by mating of cells containing the expression constructs, or by cell fusion, transformation, electroporation, microinjection, etc. In a specific embodiment in which the assay is carried out in mammalian cells (e.g., hamster cells), the DNA binding domain is the GAL4 DNA binding domain, the activation domain is the herpes simplex virus VP16 transcriptional activation domain, and the reporter gene contains the desired coding sequence operably linked to a minimal promoter element from the adenovirus E1B gene driven by several GAL4 DNA binding sites (see Fearon et al., 1992, Proc. Natl. Acad. Sci. USA 89:7958-7962). The host cell used should not express an endogenous transcription factor that binds to the same DNA site as that recognized by the DNA binding domain fusion population. Also, preferably, the host cell is mutant or otherwise lacking in an endogenous, functional form of the reporter gene(s) used in the assay.

Various vectors and host strains for expression of the two fusion protein populations in yeast are known and can be used (see, e.g., U.S. Pat. No. 5,1468,614; Bartel et al., 1993, “Using the two-hybrid system to detect protein-protein interactions” In: Cellular Interactions in Development, Hartley, D. A. (ed.), Practical Approach Series xviii, IRL Press at Oxford University Press, New York, N.Y., pp. 153-179; Fields and Sternglanz, 1994, Trends In Genetics 10:286-292). Exemplary strains that can be used in the assay of the invention also include, but are not limited to, the following:
Y190: MATa, ura3-52, his3-200, lys2-801, ade2-101, trp1-901, leu2-3,112, gal4α, gal80α, cyh^{r}2, LYS2::GAL1_{UAS}-HIS3_{TATA}HIS3, URA3::GAL1_{UAS}-GAL1_{TATA}-lacZ; Harper et al., 1993, Cell 75:805-816, available from Clontech, Palo Alto, Calif. Y190 contains HIS3 and lacZ reporter genes driven by GAL4 binding sites.
CG-1945: MATa, ura3-52, his3-200, lys2-801, ade2-101, trp1-901, leu2-3,112, gal4-542, gal80-538, cyh^{r}2, LYS2::GAL1_{UAS}-HIS3_{TATA}HIS3, URA3::GAL1_{UAS17mers(x3)}-CYC1_{TATA}-lacZ, available from Clontech, Palo Alto, Calif. CG-1945 contains HIS3 and lacZ reporter genes driven by GAL4 binding sites.
Y187: MAT-α, ura3-52, his3-200, ade2-101, trp1-901, leu2-3,112, gal4α, gal80α, URA3::GAL1_{UAS}-GAL1_{TATA}-lacZ, available from Clontech, Palo Alto, Calif. Y187 contains a lacZ reporter gene driven by GAL4 binding sites.
SFY526: MATa, ura3-52, his3-200, lys2-801, ade2-101, trp1-901, leu2-3,112, gal4-542, gal80-538, can^{r}, URA3::GAL1-lacZ, available from Clontech, Palo Alto, Calif. SFY526 contains HIS3 and lacZ reporter genes driven by GAL4 binding sites.
HF7c: MATa, ura3-52, his3-200, lys2-801, ade2-101, trp1-901, leu2-3,112, gal4-542, gal80-538, LYS2::GAL1-HIS3, URA3::GAL1_{UAS 17MERS(x3)}-CYC1-lacZ, available from Clontech, Palo Alto, Calif. HF7c contains HIS3 and lacZ reporter genes driven by GAL4 binding sites.
YRG-2: MATa, ura3-52, his3-200, lys2-801, ade2-101, trp1-901, leu2-3,112, gal4-542, gal80-538, LYS2::GAL1_{UAS}-GAL1_{TATA}-HIS3, URA3::GAL1_{UAS17mers(x3)}-CYC1-lacZ, available from Stratagene, La Jolla, Calif. YRG-2 contains HIS3 and lacZ reporter genes driven by GAL4 binding sites.

Many other strains commonly known and available in the art can be used.

If not already lacking in endogenous reporter gene activity, cells mutant in the reporter gene may be selected by known methods, or the cells can be made mutant in the target reporter gene by known gene-disruption methods prior to introducing the reporter gene (Rothstein, 1983, Meth. Enzymol. 101:202-211).

In a specific embodiment, plasmids encoding the different fusion protein populations can be introduced simultaneously into a single host cell (e.g., a haploid yeast cell) containing one or more reporter genes, by co-transformation, to conduct the assay for protein-protein interactions. Or, preferably, the two fusion protein populations are introduced into a single cell either by mating (e.g., for yeast cells) or cell fusions (e.g., of mammalian cells). In a mating type assay, conjugation of haploid yeast cells of opposite mating type that have been transformed with a binding domain fusion expression construct (preferably a plasmid) and an activation (or inhibitor) domain fusion expression construct (preferably a plasmid), respectively, will deliver both constructs into the same diploid cell. The mating type of a yeast strain may be manipulated by transformation with the HO gene (Herskowitz and Jensen, 1991, Meth. Enzymol. 194:132-146).

In a preferred embodiment, a yeast interaction mating assay is employed using two different types of host cells, strain-type a and alpha of the yeast Saccharomyces cerevisiae. The host cell preferably contains at least two reporter genes, each with one or more binding sites for the DNA-binding domain (e.g., of a transcriptional activator). The activator domain and DNA binding domain are each parts of chimeric proteins formed from the two respective populations of proteins. One strain of host cells, for example the a strain, contains fusions of the library of nucleotide sequences with the DNA-binding domain of a transcriptional activator, such as GAL4. The hybrid proteins expressed in this set of host cells are capable of recognizing the DNA-binding site in the promoter or enhancer region in the reporter gene construct. The second set of yeast host cells, for example, the alpha strain, contains nucleotide sequences encoding fusions of a library of DNA sequences fused to the activation domain of a transcriptional activator.

In a preferred embodiment, the fusion protein constructs are introduced into the host cell as a set of plasmids. These plasmids are preferably capable of autonomous replication in a host yeast cell and preferably can also be propagated in E. coli. The plasmid contains a promoter directing the transcription of the DNA binding or activation domain fusion genes, and a transcriptional termination signal. The plasmid also preferably contains a selectable marker gene, permitting selection of cells containing the plasmid. The plasmid can be single-copy or multi-copy. Single-copy yeast plasmids that have the yeast centromere may also be used to express the activation and DNA binding domain fusions (Elledge et al., 1988, Gene 70:303-312).

In another embodiment, the fusion constructs are introduced directly into the yeast chromosome via homologous recombination. The homologous recombination for these purposes is mediated through yeast sequences that are not essential for vegetative growth of yeast, e.g., the MER2, MER1, ZIP1, REC102, or ME14 gene.

Bacteriophage vectors can also be used to express the DNA binding domain and/or activation domain fusion proteins. Libraries can generally be prepared faster and more easily from bacteriophage vectors than from plasmid vectors.

In a specific embodiment, the present invention provides a method of detecting one or more protein-protein interactions comprising (a) recombinantly expressing Mam or a derivative or analog thereof in a first population of yeast cells being of a first mating type and comprising a first fusion protein containing the Mam sequence and a DNA binding domain, wherein said first population of yeast cells contains a first nucleotide sequence operably linked to a promoter driven by one or more DNA binding sites recognized by said DNA binding domain such that an interaction of said first fusion protein with a second fusion protein, said second fusion protein comprising a transcriptional activation domain, results in increased transcription of said first nucleotide sequence; (b) negatively selecting to eliminate those yeast cells in said first population in which said increased transcription of said first nucleotide sequence occurs in the absence of said second fusion protein; (c) recombinantly expressing in a second population of yeast cells of a second mating type different from said first mating type, a plurality of said second fusion proteins, each second fusion protein comprising a sequence of a fragment, derivative or analog of a Mam-IP and an activation domain of a transcriptional activator, in which the activation domain is the same in each said second fusion protein; (d) mating said first population of yeast cells with said second population of yeast cells to form a third population of diploid yeast cells, wherein said third population of diploid yeast cells contains a second nucleotide sequence operably linked to a promoter driven by a DNA binding site recognized by said DNA binding domain such that an interaction of a first fusion protein with a second fusion protein results in increased transcription of said second nucleotide sequence, in which the first and second nucleotide sequences can be the same or different; and (e) detecting said increased transcription of said first and/or second nucleotide sequence, thereby detecting an interaction between a first fusion protein and a second fusion protein.

In a preferred embodiment, the bait Mam sequence and the prey library of chimeric genes are combined by mating the two yeast strains on solid media for a period of approximately 6-8 hours. In a less preferred embodiment, the mating is performed in liquid media. The resulting diploids contain both kinds of chimeric genes, i.e., the DNA-binding domain fusion and the activation domain fusion.

Preferred reporter genes include the URA3, HIS3 and/or the lacZ genes (see, e.g., Rose and Botstein, 1983, Meth. Enzymol. 101:167-180) operably linked to GAL4 DNA-binding domain recognition elements. Other reporter genes comprise the functional coding sequences for, but not limited to, Green Fluorescent Protein (GFP) (Cubitt et al., 1995, Trends Biochem. Sci. 20:448-455), luciferase, LEU2, LYS2, ADE2, TRP1, CAN1, CYH2, GUS, CUP1 or chloramphenicol acetyl transferase (CAT). Expression of LEU2, LYS2, ADE2 and TRP1 are detected by growth in a specific defined media; GUS and CAT can be monitored by well known enzyme assays; and CAN1 and CYH2 are detected by selection in the presence of canavanine and cycloheximide. With respect to GFP, the natural fluorescence of the protein is detected.

In a specific embodiment, transcription of the reporter gene is detected by a linked replication assay. For example, as described by Vasavada et al., 1991, Proc. Natl. Acad. Sci. USA 88:10686-10690, expression of SV40 large T antigen is under the control of the E1B promoter responsive to GAL4 binding sites. The replication of a plasmid containing the SV40 origin of replication, indicates the reconstruction of the GAL4 protein and a protein-protein interaction. Alternatively, a polyoma virus replicon can be employed (Vasavada et al., 1991, Proc. Natl. Acad. Sci. USA 88:10686-10690).

In another embodiment, the expression of reporter genes that encode proteins can be detected by immunoassay, i.e., by detecting the immunospecific binding of an antibody to such protein, which antibody can be labeled, or alternatively, which antibody can be incubated with a labeled binding partner to the antibody, so as to yield a detectable signal. Alam and Cook (1990, Anal. Biochem. 188:245-254) disclose non-limiting examples of detectable marker genes that can be operably linked to a transcriptional regulatory region responsive to a reconstituted transcriptional activator, and thus used as reporter genes.

The activation of reporter genes like URA3 or HIS3 enables the cells to grow in the absence of uracil or histidine, respectively, and hence serves as a selectable marker. Thus, after mating, the cells exhibiting protein-protein interactions are selected by the ability to grow in media lacking a nutritional component, such as uracil or histidine (referred to as -URA (minus URA) and -HIS (minus HIS) medium, respectively). The -HIS medium preferably contains 3-amino-1,2,4-triazole (3-AT), which is a competitive inhibitor of the HIS3 gene product, and thus, requires higher levels of transcription in the selection (see, Durfee et al., 1993, Genes Dev. 7:555-569). Similarly, 6-azauracil, which is an inhibitor of the URA3 gene product, can be included in -URA medium (Le Douarin et al., 1995, Nucl. Acids Res. 23:876-878). URA3 gene activity can also be detected and/or measured by determining the activity of its gene product, orotidine-51-monophosphate decarboxylase (Pierrat et al., 1992, Gene 119:237-245, Wolcott et al., 1966, Biochem. Biophys. Acta 122:532-534). In other embodiments of the present invention, the activities of the reporter genes like GFP or lacZ are monitored by measuring a detectable signal (e.g., fluorescent or chromogenic, respectively) that results from the activation of these reporter genes. For example, lacZ transcription can be monitored by incubation in the presence of a chromogenic substrate, such as X-gal (5-bromo-4-chloro-3-indolyl-β-D-galactoside), of its encoded enzyme, β-galactosidase. The pool of all interacting proteins isolated by this manner from mating the Mam sequence product and the library identifies the “Mam interactive population”.

In a preferred embodiment of the present invention, false positives arising from transcriptional activation by the DNA binding domain fusion proteins in the absence of a transcriptional activator domain fusion protein are prevented or reduced by negative selection for such activation within a host cell containing the DNA binding fusion population, prior to exposure to the activation domain fusion population. By way of example, if such cell contains URA3 as a reporter gene, negative selection is carried out by incubating the cell in the presence of 5-fluoroorotic acid (5-FOA, which kills URA+ cells (Rothstein, 1983, Meth. Enzymol. 101:167-180). Hence, if the DNA-binding domain fusions by themselves activate transcription, the metabolism of 5-FOA will lead to cell death and the removal of self-activating DNA-binding domain hybrids.

Negative selection involving the use of a selectable marker as a reporter gene and the presence in the cell medium of an agent toxic or growth inhibitory to the host cells in the absence of reporter gene transcription is preferred, since it allows a higher rate of processing than other methods. As will be apparent, negative selection can also be carried out on the activation domain fusion population prior to interaction with the DNA binding domain fusion population, by similar methods, either alone or in addition to negative selection of the DNA binding fusion population.

Negative selection can also be carried out on the recovered Mam:Mam-IP complex by known methods (see, e.g., Bartel et al., 1993, BioTechniques 14:920-924) although pre-negative selection (prior to the interaction assay), as described above, is preferred. For example, each plasmid encoding a protein (peptide or polypeptide) fused to the activation domain (one-half of a detected interacting complex) can be transformed back into the original screening strain, either alone or with a plasmid encoding only the DNA-binding domain, the DNA-binding domain fused to the detected interacting protein, or the DNA-binding domain fused to a protein that does not affect transcription or participate in the protein-protein interaction. A positive interaction detected with any plasmid other than that encoding the DNA-binding domain fusion to the detected interacting protein is deemed a false positive and is eliminated from the screen.

In a preferred embodiment, the Mam plasmid population is transformed in a yeast strain of a first mating type (a or alpha), and the second plasmid population (containing the library of DNA sequences) is transformed in a yeast strain of a different mating type. Both strains are preferably mutant for URA3 and HIS3, and contain HIS3, and optionally lacZ, as reporter genes. The first set of yeast cells are positively selected for the Mam plasmids and are negatively selected for false positives by incubation in medium lacking the selectable marker (e.g., tryptophan) and containing 5-FOA. Yeast cells of the second mating type are transformed with the second plasmid population, and are positively selected for the presence of the plasmids containing the library of fusion proteins. Selected cells are pooled. Both groups of pooled cells are mixed together and mating is allowed to occur on a solid phase. The resulting diploid cells are then transferred to selective media that selects for the presence of each plasmid and for activation of reporter genes.

In a preferred embodiment of the invention, after an interactive population is obtained, the DNA sequences encoding the pairs of interactive proteins are isolated by a method wherein either the DNA-binding domain hybrids or the activation domain hybrids are amplified, in separate respective reactions. Preferably, the amplification is carried out by polymerase chain reaction (PCR) (see, U.S. Pat. Nos. 4,683,202; 4,683,195; and 4,889,818; Gyllenstein et al., 1988, Proc. Natl. Acad. Sci. USA 85:7652-7656; Ochman et al., 1988, Genetics 120:621-623; Loh et al., 1989, Science 243:217-220; Innis et al., 1990, PCR Protocols, Academic Press, Inc., San Diego, Calif.) using pairs of oligonucleotide primers specific for either the DNA-binding domain hybrids or the activation domain hybrids. This PCR reaction can also be performed on pooled cells expressing interacting protein complexes, preferably pooled arrays of interactants. Other amplification methods known in the art can be used, including but not limited to ligase chain reaction (see EP 320,308), use of Qβ replicase, or methods listed in Kricka et al., 1995, Molecular Probing, Blotting, and Sequencing, Academic Press, New York, Chapter 1 and Table IX.

The plasmids encoding the DNA-binding domain hybrid and the activation domain hybrid proteins can also be isolated and cloned by any of the methods well known in the art. For example, but not by way of limitation, if a shuttle (yeast to E. coli) vector is used to express the fusion proteins, the genes can be recovered by transforming the yeast DNA into E. coli and recovering the plasmids from E. coli (see, e.g., Hoffman et al., 1987, Gene 57:267-272). Alternatively, the yeast vector can be isolated, and the insert encoding the fusion protein subcloned into a bacterial expression vector, for growth of the plasmid in E. coli.

5.9 Pharmaceutical Compositions and Therapeutic/Prophylactic Administration

The invention provides methods of treatment (and prophylaxis) by administration to a subject of an effective amount of a Therapeutic of the invention. In a preferred aspect, the Therapeutic is substantially purified. The subject is preferably an animal including, but not limited to animals such as cows, pigs, horses, chickens, cats, dogs, etc., and is preferably a Mammal, and most preferably human. In a specific embodiment, a non-human Mammal is the subject.

Formulations and methods of administration that can be employed when the Therapeutic comprises a nucleic acid are described in Sections 5.5.2 and 5.5.3, supra; additional appropriate formulations and routes of administration can be selected from among those described herein below.

Various delivery systems are known and can be used to administer a Therapeutic of the invention, e.g., encapsulation in liposomes, microparticles, and microcapsules: use of recombinant cells capable of expressing the Therapeutic, use of receptor-mediated endocytosis (e.g., Wu and Wu, 1987, J. Biol. Chem. 262:4429-4432); construction of a Therapeutic nucleic acid as part of a retroviral or other vector, etc. Methods of introduction include but are not limited to intradermal, intramuscular, intraperitoneal, intravenous, subcutaneous, intranasal, epidural, and oral routes. The compounds may be administered by any convenient route, for example by infusion, by bolus injection, by absorption through epithelial or mucocutaneous linings (e.g., oral, rectal and intestinal mucosa, etc.), and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment. This may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

In another embodiment, the Therapeutic can be delivered in a vesicle, in particular a liposome (Langer, 1990, Science 249:1527-1533; Treat et al., 1989, In: Liposomes in the Therapy of Infectious Disease and Cancer, Lopez-Berestein and Fidler, eds., Liss, New York, pp. 353-365; Lopez-Berestein, ibid., pp. 317-327; see generally ibid.)

In yet another embodiment, the Therapeutic can be delivered via a controlled release system. In one embodiment, a pump may be used (Langer, supra; Sefton, 1987, CRC Crit. Ref. Biomed. Eng. 14:201-240; Buchwald et al., 1980, Surgery 88:507-516; Saudek et al., 1989, N. Engl. J. Med. 321:574-579). In another embodiment, polymeric materials can be used (Medical Applications of Controlled Release, Langer and Wise, eds., CRC Press, Boca Raton, Fla., 1974; Controlled Drug Bioavailability, Drug Product Design and Performance, Smolen and Ball, eds., Wiley, New York, 1984; Ranger and Peppas, 1983, Macromol. Sci. Rev. Macromol. Chem. 23:61; Levy et al., 1985, Science 228:190-192; During et al., 1989, Ann. Neurol. 25:351-356; Howard et al., 1989, J. Neurosurg. 71:858-863). In yet another embodiment, a controlled release system can be placed in proximity of the therapeutic target, i.e., the brain, thus requiring only a fraction of the systemic dose (e.g., Goodson, 1984, In: Medical Applications of Controlled Release, supra, Vol. 2, pp. 115-138). Other controlled release systems are discussed in the review by Langer (1990, Science 249:1527-1533).

In a specific embodiment where the Therapeutic is a nucleic acid encoding a protein Therapeutic, the nucleic acid can be administered in vivo to promote expression of its encoded protein, by constructing it as part of an appropriate nucleic acid expression vector and administering it so that it becomes intracellular, e.g., by use of a retroviral vector (U.S. Pat. No. 4,980,286), or by direct injection, or by use of microparticle bombardment (e.g., a gene gun; Biolistic, Dupont), or by coating it with lipids, cell-surface receptors or transfecting agents, or by administering it in linkage to a homeobox-like peptide which is known to enter the nucleus (e.g., Joliot et al., 1991, Proc. Natl. Acad. Sci. USA 88:1864-1868), etc. Alternatively, a nucleic acid Therapeutic can be introduced intracellularly and incorporated by homologous recombination within host cell DNA for expression.

The present invention also provides pharmaceutical compositions. Such compositions comprise a therapeutically effective amount of a Therapeutic, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water is a preferred carrier when the pharmaceutical composition is administered orally. Saline and aqueous dextrose are preferred carriers when the pharmaceutical composition is administered intravenously. Saline solutions and aqueous dextrose and glycerol solutions are preferably employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the Therapeutic, preferably in purified form, together with a suitable amount of carrier so as to provide the form for proper administration to the patient. The formulation should suit the mode of administration.

In a preferred embodiment, the composition is formulated, in accordance with routine procedures, as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water or saline for injection can be provided so that the ingredients may be mixed prior to administration.

The Therapeutics of the invention can be formulated as neutral or salt forms. Pharmaceutically acceptable salts include those formed with free carboxyl groups such as those derived from hydrochloric, phosphoric, acetic, oxalic, tartaric acids, etc., those formed with free amine groups such as those derived from isopropylamine, triethylamine, 2-ethylamino ethanol, histidine, procaine, etc., and those derived from sodium, potassium, ammonium, calcium, and ferric hydroxides, etc.

The amount of the Therapeutic of the invention which will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Suppositories generally contain active ingredient in the range of 0.5% to 10% by weight; oral formulations preferably contain 10% to 95% active ingredient.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

5.10 Animal Models

The present invention also provides animal models. In one embodiment, animal models for diseases and disorders involving Mam:Mip1, Mam:Mip30 or Mam:Mip6 complexes are provided. These include, but are not limited to, disease or disorders of cell fate and differentiation and disorders associated with aberrant mitosis, see Section 2, supra. Such animals can be initially produced by promoting homologous recombination or insertional mutagenesis between Mam, Mip1, Mip30 and/or Mip6 genes in the chromosome, and exogenous Mam, Mip1, Mip30 and/or Mip6 genes that have been rendered biologically inactive or deleted (preferably by insertion of a heterologous sequence, e.g., an antibiotic resistance gene). In a preferred aspect, homologous recombination is carried out by transforming embryo-derived stem (ES) cells with a vector containing the insertionally inactivated Mam, Mip1, Mip30 and/or Mip6 genes, such that homologous recombination occurs, followed by injecting the transformed ES cells into a blastocyst, and implanting the blastocyst into a foster mother, followed by the birth of the chimeric animal (“knockout animal”) in which a Mam, Mip1, Mip30 and/or Mip6 gene has been inactivated or deleted (Capecchi, 1989, Science 244:1288-1292). In another preferred aspect, site-specific recombinases can be used, such as cre which recognizes lox sites and flp which recognizes frt sites. The chimeric animal can be bred to produce additional knockout animals. Such animals can be mice, hamsters, sheep, pigs, cattle, etc., and are preferably non-human Mammals. In a specific embodiment, a knockout mouse is produced.

Such knockout animals are expected to develop, or be predisposed to developing, diseases or disorders involving, but not restricted to, diseases and disorder of cell fate and differentiation, and a number of less common syndromes and disorders associated with aberrant mitotic events, and thus, can have use as animal models of such diseases and disorders, e.g., to screen for or test molecules (e.g., potential Therapeutics) for diseases or disorders of cell fate and differnentiation, e.g., hyperproliferative disorders and malignacies.

In a different embodiment of the invention, transgenic animals that have incorporated and express (or overexpress or mis-express) a functional Mam, Mip1, Mip30 and/or Mip6 gene, e.g. by introducing the Mam and Mip1 genes under the control of a heterologous promoter (i.e., a promoter that is not the native Mam or Mip1 promoter) that either overexpresses the protein or proteins, or expresses them in tissues not normally expressing the complexes or proteins, can have use as animal models of diseases and disorders characterized by elevated levels of Mam:Mip1 complexes. Such animals can be used to screen or test molecules for the ability to treat or prevent the diseases and disorders cited supra.

In one embodiment, the present invention provides a recombinant non-human animal in which both an endogenous Mam gene and an endogenous Mip1 have been deleted or inactivated by homologous recombination or insertional mutagenesis of said animal or an ancestor thereof. In another embodiment, the invention provides a recombinant non-human animal containing both a Mam gene and a Mip1 gene in which the Mam gene is under the control of a promoter that is not the native Mam gene promoter and the Mip1 gene is under the control of a promoter that is not the native Mip 1 gene promoter. In a specific embodiment, the invention provides a recombinant non-human animal containing a transgene comprising a nucleic acid sequence encoding a chimeric protein comprising a fragment of Mam of at least 6 amino acids fused via a covalent bond to a fragment of Mip1 protein of at least 6 amino acids.

### 6. EXAMPLES

6.1 Identification of Mam Interactions

To elucidate the function of Mam and its role in Notch signaling, proteins with which Mam interacts were identified. Complementary DNA encoding a truncated Mam protein, in which the carboxy-terminal 32 amino acids of full-length Mam were deleted, was fused to the Gal4 DNA-binding domain encoded by the yeast expression vector pEG202. A truncated Mam fusion protein was used because it elicited a lower autonomous transactivational response from yeast reporter genes than full-length Mam. Using this fusion protein as bait, approximately 3×10⁶ yeast transformants expressing proteins encoded by Drosophila cDNAs were screened, prepared from 0-12 hour embryos, fused to the E. coli B42 transactivation domain encoded by pJG4-5. FIG. 13 is a graph showing the results of a yeast two-hybrid screen deomonstrating that Mam interacts with Mip1, Mip30 and Mip6.

Three cDNAs encoding Mam-Interacting Proteins (Mips) were isolated. The largest cDNA encoding one of the interacting proteins, Mip1, was 862 nucleotides in length, included 19 poly (A) residues at its 3′ end, and predicted an amino-terminally truncated protein of 242 amino acids. To obtain additional 5′ sequence, the Mip1 cDNA isolated from the two-hybrid library was used as a probe to screen a lambda phage cDNA library prepared from 0-14 hour embryos. The largest Mip1 cDNA clone isolated from this library was 2072 nucleotides in length, was polyadenylated at an identical position, and encoded a protein of 411 amino acids that also appeared to be truncated at its amino terminus. The remaining amino-terminal sequence of the Mip1 protein was identified in the sequences of EST and baculoviris clones, deposited in GenBank, and was isolated by PCR from a lambda phage library. The entire Mip1 cDNA is 2348 nucleotides in length, and the largest open reading frame encodes a protein of 700 amino acids with a predicted molecular mass of 78 kD. The size of this cDNA agrees closely with the size of the single transcript detected by northern blot analysis.

6.2 Characterization of Mip1, Mip30 and Mip6

A search of the Mip1 protein sequence for profiles or patterns using the InterPro Scan program identified three prominent signature motifs. These are an UBA/THIF-type NAD/FAD binding fold (THIF family), an ubiquitin-activating enzyme repeat domain (UBACT repeat), and a bipartite nuclear localization signal (FIG. 5). Each of these motifs is evolutionarily conserved and found in organisms ranging from human to bacteria (THIF family) or yeast (UBACT family). In eukaryotes, these motifs are present in ubiquitin-activating enzymes (E1-type enzymes). E1-type enzymes activate ubiquitin or ubiquitin-related proteins, first by adenylating a C-terminal glycine residue with AT2, and then by forming a thicester linkage between the ubiquitin or ubiquitin-related protein and a cysteine residue of the E1 enzyme, releasing AMP. The ubiquitin or ubiquitin-related moiety is subsequently serially transferred to a cysteine residue of an ubiquitin-conjugating enzyme (E2 enzyme), a cysteine residue of a ubiquitin ligase, and then ultimately a lysine residue of a target protein.

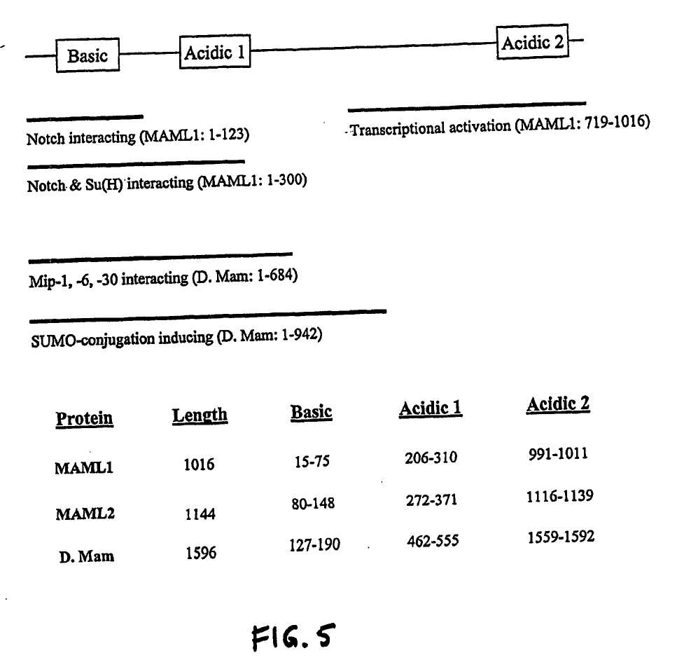

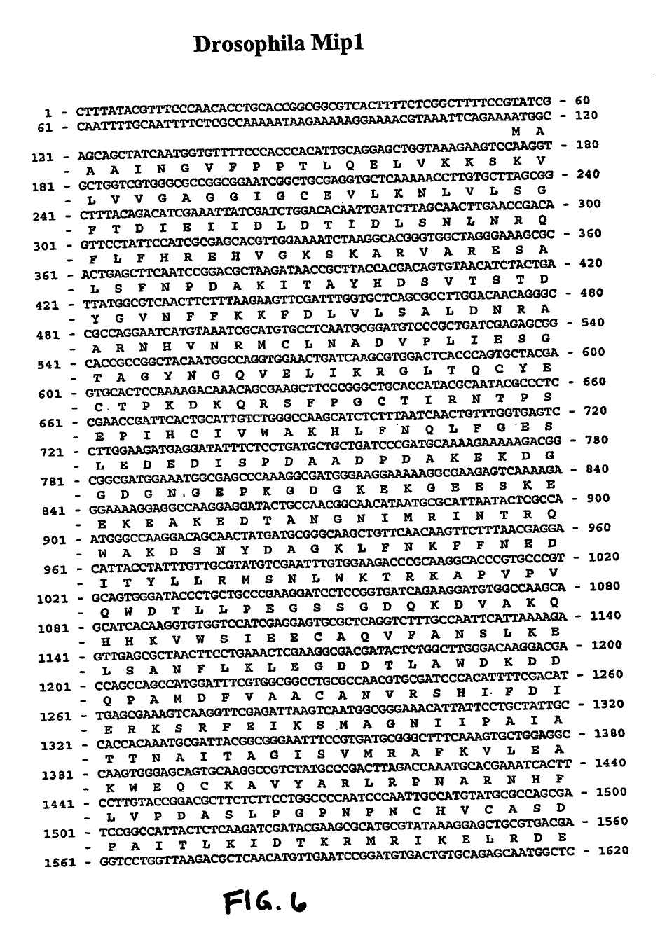

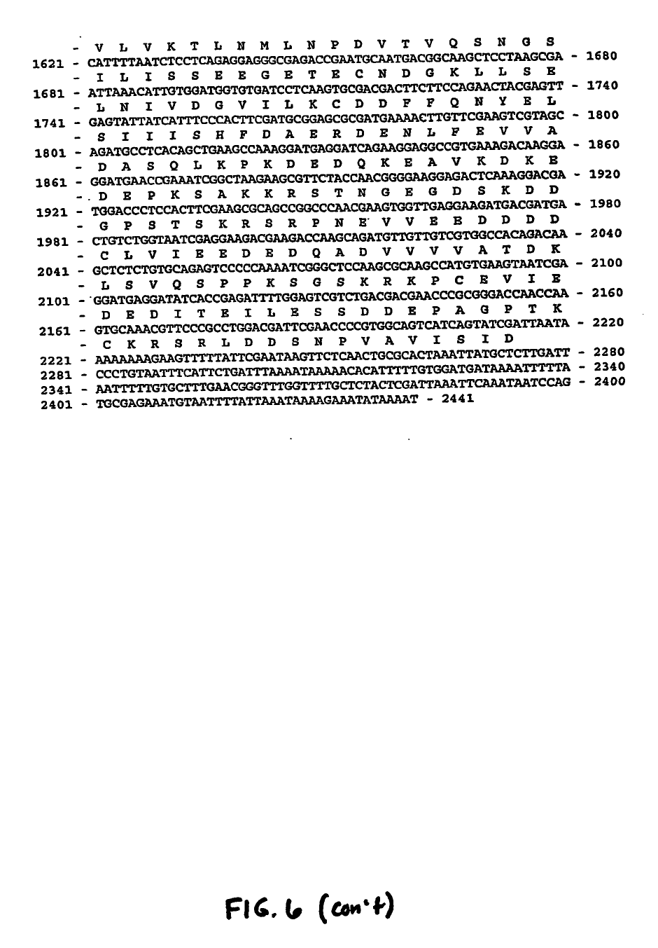

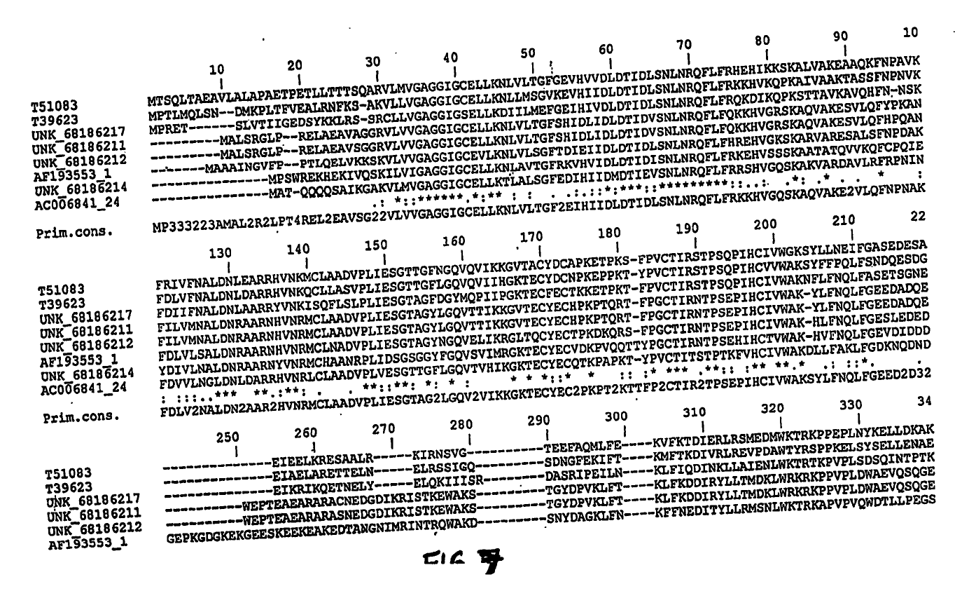

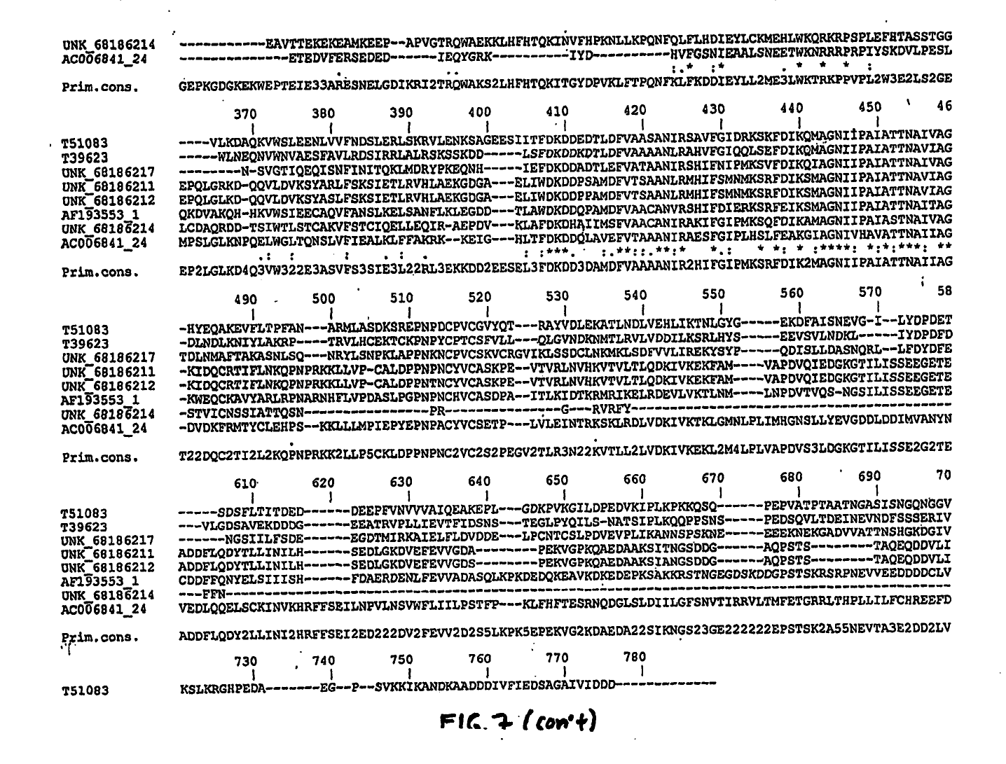

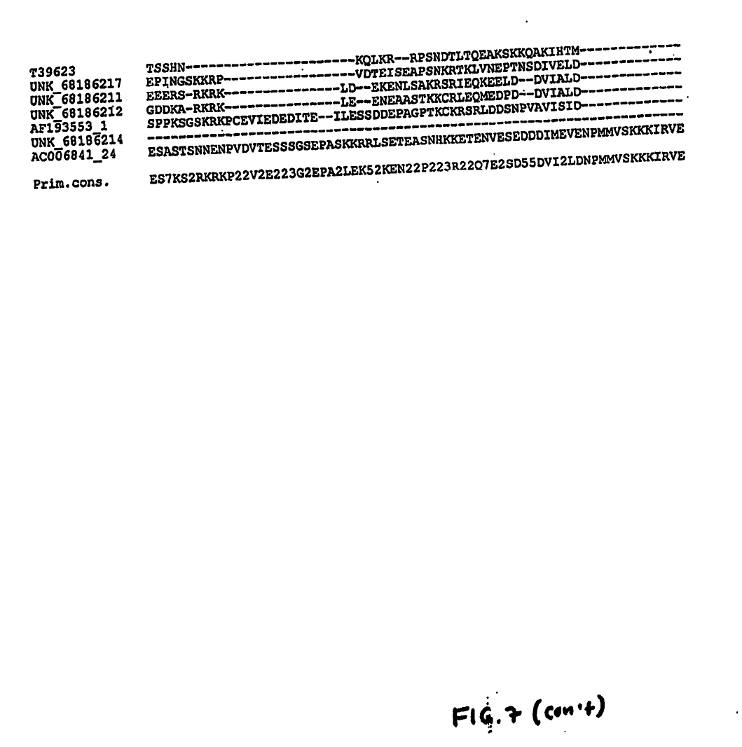

Using the BLAST program to search GenBank for sequence similarities revealed that Mip1 is the Drosophila ubiquitin-like activating enzyme Uba2p. This protein is one subunit of a heterodixneric E1-type enzyme that activates the Small Ubiquitin-related Modifier, SUMO/Smt3, and is extremely well conserved throughout evolution. An alignment of the Drosophila and human proteins using the CUSTALW program indicates that they are 46% identical (FIG. 7).

The SUMO-conjugation machinery appears to be present in all Eukaryota and mechanistically parallels the ubiquitin-conjugation machinery. The notable differences are that the E1 enzyme of the ubiquitin-conjugation pathway is composed of a single protein, while the E1 enzyme of the SUMO-conjugation pathway is composed of two subunits: Aos1p (another THIF-family protein) and Uba2p (Mip1). These two proteins are equivalent to the amino- and carboxy-terminal regions of the classic ubiquitin E1 enzyme, respectively. Additionally, an E3-type protein ligase has not yet been identified as a component of the SUMO-conjugation machinery.

The cDNA encoding Mip30, isolated from the two-hybrid screen, was 1818 nucleotides in length, included 18 poly(A) residues at its 3′ end, and predicted an amino-terminally truncated protein of 315 amino acids. To obtain additional 5′ sequence, the Mip30 cDNA isolated from the two-hybrid library was used as a probe to screen a lambda phage cDNA library. An additional 2560 nucleotides of 5′ sequence was obtained. The remaining 5′ sequence (nucleotides 1-7) of Mip30 was identified in the sequence of EST clones (AA948812, AA940874 and AA949252) deposited in GenBank. The entire Mip30 sequence is 2567 nucleotides in length, and the largest open reading frame encodes a protein of 543 amino acids with a predicted molecular mass of 63 kD, corresponding to the protein predicted by AF132187. Sequence analyses of lambda phage clones revealed three forms of Mip30 cDNA that differ in the length of their 3′-untranslated regions. The predicted size of the transcripts agrees closely with the sizes predicted by Northern blot analysis. A search of the Mip30 protein sequence for profiles or patterns using the InterPro Scan program identified three prominent signature motifs. These are seven C2H2-type zinc fingers, an HMG-1 and HMG-Y DNA-binding domain (A+T-hook), and a bipartite nuclear localization signal. See FIG. 11.

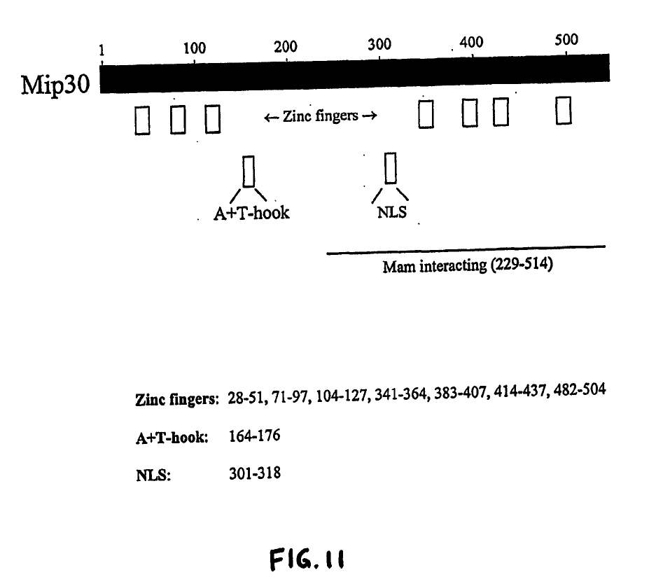

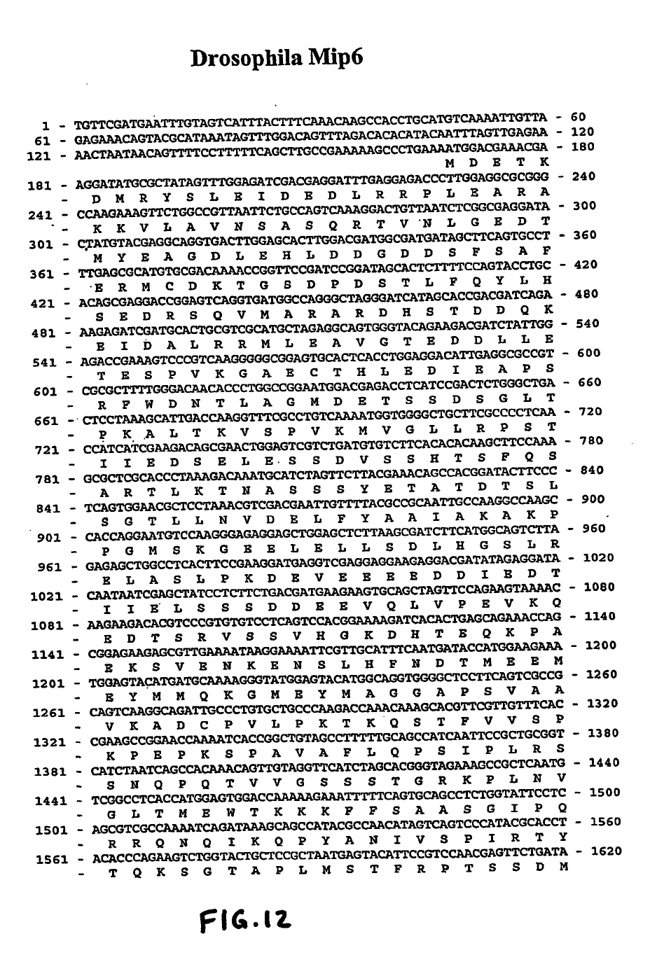

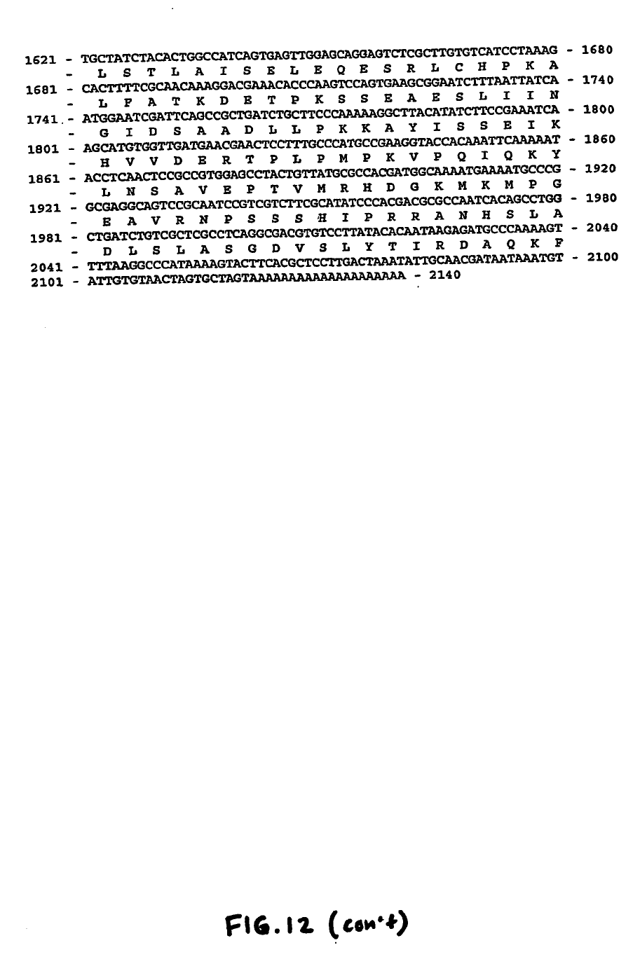

The cDNA encoding Mip6, isolated from the two-hybrid screen, was 1224 nucleotides in length, included 20 poly(A) residues at its 3′ end, and predicted an amino-terminally truncated protein of 251 amino acids. To obtain additional 5′ sequence, the Mip6 cDNA isolated from the two-hybrid library was used as a probe to screen a lambda phage cDNA library. An additional 35 nucleotides (11 amino acids) of 5′ sequence was obtained from this clone. The remaining amino-terminal sequence of the Mip1 protein was identified in the sequence of EST (BF503916) and baculovirus (AC008326 and AC007977) clones, as well as the Drosophila genomic scaffold (AE003615), deposited in GenBank. The entire Mip6 sequence is 2140 nucleotides in length, and the largest open reading frame encodes a protein of 625 amino acids with a predicted molecular mass of 69 kD, corresponding to the conceptual translation AAF52468. The size of this cDNA agrees closely with the size of the single transcript detected by northern blot analysis. The only identifiable motif in the Mip6 protein is a bipartite nuclear localization signal (amino acids 420-437). See FIG. 12.

6.3 Subcellular Localization of Mastermind

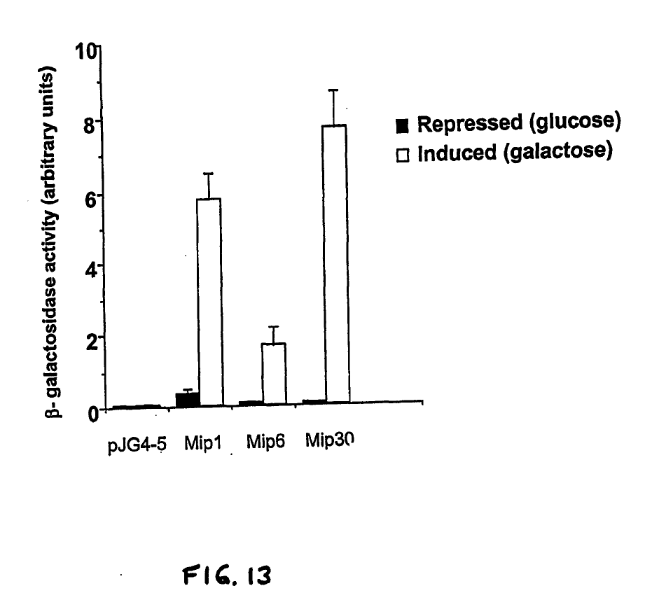

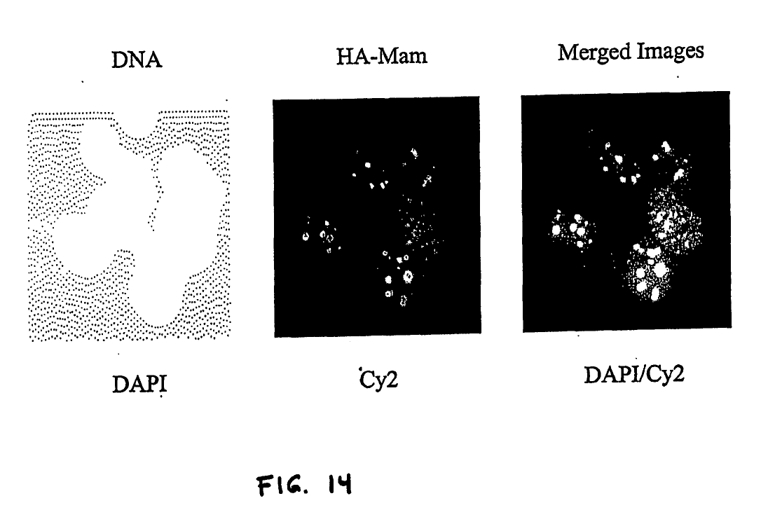

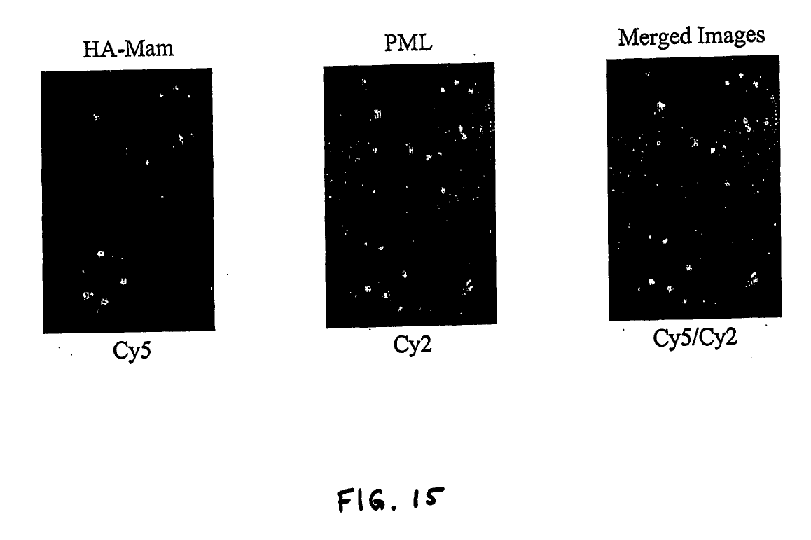

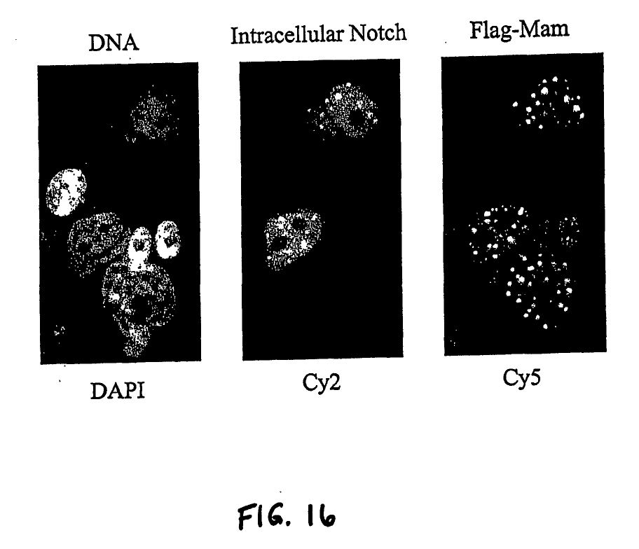

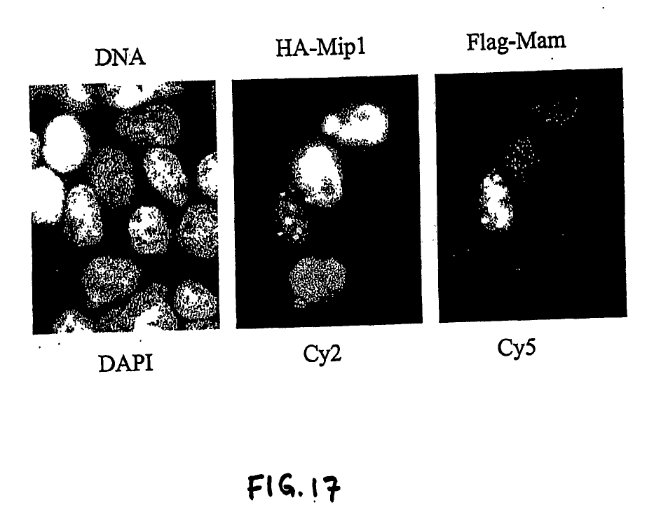

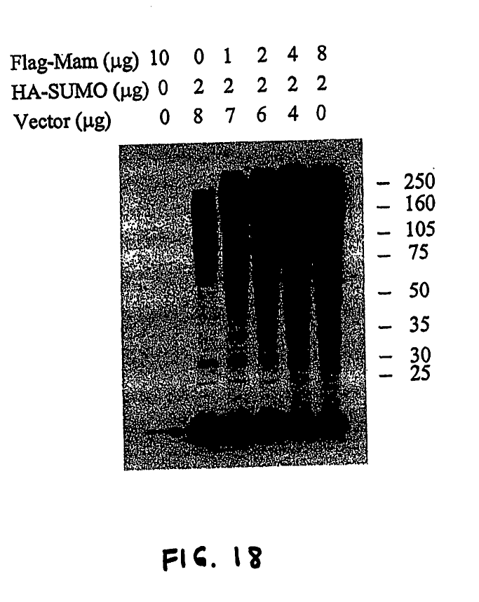

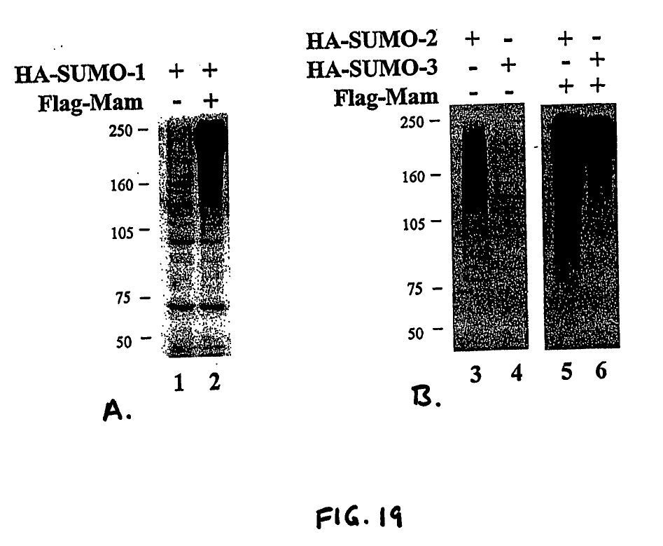

Indirect immunofluorescence analysis of human 293T cells, Drosophila S2 cells or Spodoptera SF9 cells transiently transfected with Drosophila Mastermind (Mam), epitope-tagged at its amino terminus with either Flag or hemagglutinin (HA) revealed that Mam localizes to discrete subnuclear domains (FIG. 14). A similar subnuclear localization has been found for a human protein, MAML1, that shares limited homology with Drosophila Mam. Co-localization in 293T cells with antibodies to the promyelocytic leukemia (PML.) protein identified these domains as nuclear bodies (NBs) (FIG. 15). Nuclear bodies (also called PML bodies, PODs or ND10) are general features of cells; there is typically 5-20 per cell, they range in size from 0.1-1μ, and they are spherical or toroidal in shape. Many types of proteins have been found to co-localize with NBs, including transcription factors and coactivators, chromosomal proteins, tumor suppressors and proto-oncogenes. Interestingly, the SUMO protein has been shown to be associated with NBs, and components of NBs, such as the signature proteins PML and SP100, are conjugated to SUMO, see Section 2, supra. A relationship between NBs and disease is exemplified by their disruption in malignancies, such as acute promyelocytic leukemia, and upon viral infection. The function of NBs is still unknown, but based upon the variety of proteins that are found associated with NBs, two favored hypotheses are that these structures are sites for signal integration or sites for protein storage/removal.

The localization of Mam to NBs indicated that Mam recruits other components of the Notch pathway to these structures. Co-expression of epitope-tagged Mam and intracellular Notch in 293T cells demonstrated that Mam causes the relocalization of intracellular Notch from the nucleoplasm to NBs (FIG. 16). A similar observation has been reported for the human MAML1 protein, and studies have shown that mammalian Mam-like proteins as well as the C. elegans LAG-3 protein, a polyglutamine rich protein, interact with the ankyrin repeats of Notch (Wu et al., 2000, Nature Genetics 26:484-489). Furthermore, these proteins form a ternary complex with the intracellular domain of Notch and the downstream effector Su(H)/RBP-j/LAG1, and function as activators of Notch signal transduction. Taken together, these observations indicate a functional relationship between NBs and Notch signaling.

6.4 Mastermind Recruits Mip1 to NBs

Co-expression of an epitope-tagged Mam protein with an epitope-tagged Mip1 protein, in transiently transfected 293T cells, demonstrated that Mam recruits Mip1 to NBs (FIG. 17). When Mip1 is expressed alone, its distribution appears to be homogenous throughout the nucleoplasm; however, when co-expressed with Mam, Mip1 can be seen in discrete subnuclear domains. This observation confirms the interaction between Mam and Mip1 observed in the two-hybrid screen and suggests that Mam and, consequently, Notch signaling integrates into the SUMO-conjugation pathway.

6.5 Mam Activates SUMO Conjugation

Two possible scenarios can be envisioned to explain the interaction between Mam and a component of the SUMO-conjugation pathway. One scenario would have Mam as a target of the SUMO conjugation machinery, but we have not yet identified a SUMO-conjugated form of Mam. This could be explained by the observations that certain SUMO conjugates appear to be very unstable and that only a small percentage of certain target proteins exist in a conjugated form. However, most proteins that are conjugated to SUMO are found to directly interact with the SUMO conjugating enzyme (Ubc9) or the SUMO protein itself, and we have not observed such interactions. A second scenario would have Mam influence the activity of the SUMO-conjugation machinery. Given that Mam interacts with the most upstream enzyme of the SUMO-conjugating apparatus and influences the intracellular distribution of one of its subunits, we sought to test this possibility.

To investigate a potential role of Mam in SUMO conjugation, we exploited the extremely high evolutionary conservation of the SUMO conjugation machinery and the high transfectability of 293T cells. Complementary DNA encoding Drosophila SUMO was isolated from a lambda phage library by PCR, fused at its amino terminus to sequence encoding the HA epitope and cloned into an expression vector. When expressed in 293T cells, Drosophila SUMO was efficiently conjugated to cellular proteins, but at a low level. However, when Drosophila SUMO was co-expressed with Drosophila Mam, a dramatic increase in the extent of SUMO conjugation to cellular targets was observed. Furthermore, this increase was directly proportional to the amount of Mam-encoding plasmid that was introduced into these cells (FIG. 18).

As a corollary to the above experiment, we cloned cDNA encoding Drosophila SUMO and Mam into a baculovirus production vector, pFastBac, to efficiently express these protein in insect cells. Again, we found that Drosophila SUMO was efficiently conjugated to SF9 cellular proteins and the extent of conjugation was proportional to the amount of Mam expressed.

These results demonstrate that Mam can positively regulate the SUMO conjugation machinery. Taken in light of the interaction data, these observations also indicate that the activity of Mam resides at the highest level of the SUMO-conjugating apparatus.

6.6 Mam is a General Activator of the SUMO Conjugation Machinery

The genome of Drosophila predicts only one SUMO-encoding gene. However, at least three SUMO encoding genes have been identified in the genomes of mammals (SUMO1/SUMOC, SUMO2/SUMOA, and SUMO3/SUMOB). Nucleic and amino acid sequences for the human homologs of SUMOA, SUMOB, and SUMOC are found in GenBank under Accession Nos. X99584, X99585, and X99586, respectively. Drosophila SUMO is more closely related to mammalian SUMO2. We, therefore, wished to determine whether the activity of Mam upon the SUMO conjugating machinery was specific to one SUMO class or was general. Accordingly, PCR was used to isolate, clone, and epitope tag cDNA encoding each of the mammalian SUMO proteins. Co-expression of these cDNAs with Drosophila Mam revealed that Mam increases the conjugation of each of the mammalian SUMO proteins to cellular targets (FIG. 19). Therefore, Mam appears to impinge upon the SUMO conjugation machinery in a general manner.

All references cited herein are incorporated herein by reference in their entirety and for all purposes to the same extent as if each individual publication or patent or patent application was specifically and individually indicated to be incorporated by reference in its entirety for all purposes.

Many modifications and variations of the present invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

1. A method of inhibiting Notch signal transduction in a cell comprising contacting the cell with an antagonist of sumolation in an amount sufficient to inhibit Notch signal transduction.

2. A method of agonizing Notch signal transduction in a cell comprising contacting the cell with an agonist of sumolation in an amount sufficient to agonize Notch signal transduction.

3. The method according to claim 1 in which the antagonist is a dominant negative form of Mip1.

4. The method according to claim 3 in which the dominant negative form of Mip1 contains a mutated ADP binding site such that the dominant negative form of Mip1 does not bind ADP.

5. The method according to claim 1 in which the antagonist is an antisense nucleic acid to Mip1, or an antibody to Mip1 or the binding domain of an antibody to Mip1.

6. A method of identifying a molecule that alters Notch signal transduction in a cell comprising the following steps in the order stated:
(a) contacting the cell with one or more candidate molecules; and
(b) measuring the amount of sumolation in the cell,
wherein an increase or decrease in the amount of sumolation relative to said amount in a cell not so contacted with one or more of the candidate molecules indicates that the candidate molecules alter Notch signal transduction.

7. (canceled)

8. (canceled)

9. A method of identifying a molecule that alters sumolation activity in a cell comprising the following steps in the order stated:
(a) contacting the cell with one or more candidate molecules; and
(b) measuring the amount of Notch signal transduction in the cell,
wherein an increase or decrease in the amount of Notch signal transduction relative to said amount in a cell not so contacted with one or more of the candidate molecules indicates that the candidate molecules alter sumolation activity.

10. (canceled)

11. (canceled)

12. A method of inhibiting sumolation activity in a cell comprising contacting the cell with an antagonist of Notch signal transduction in an amount sufficient to inhibit sumolation activity.

13. A method of agonizing sumolation activity in a cell comprising contacting the cell with an agonist of Notch signal transduction in an amount sufficient to agonize sumolation activity.

14. The method according to claim 12 in which the antagonist is a dominant negative form of Notch.

15. The method according to claim 12 in which the antagonist is an antibody to Notch or a fragment of the antibody containing the binding domain of the antibody.

16. The method according to claim 13 in which the agonist is an dominant active form of Notch.

17. The method according to claim 13 in which the agonist is a Delta or Serrate protein or a fragment of Delta or Serrate that binds to Notch.

18. The method according to claim 13 in which the agonist is the soluble extracellular domain of Delta.

19. (canceled)

20. A purified complex of Mam and Mip1, or a purified complex of Mam and Mip30, or a purified complex of Mam and Mip6.

21. (canceled)

22. A purified complex selected from the group consisting of a complex of a derivative of Mam and Mip1, a complex of Mam and a derivative of Mip1, and a complex of a derivative of Mam and a derivative of Mip1; in which the derivative of Mam is able to form a complex with a wild-type Mip1 and the derivative of Mip1 is able to form a complex with wild-type Mam.

23. (canceled)

24. (canceled)

25. (canceled)

26. A chimeric protein comprising a fragment of Mam consisting of at least 6 amino acids fused via a covalent bond to a fragment of Mip1 consisting of at least 6 amino acids.

27. (canceled)

28. (canceled)

29. (canceled)

30. (canceled)

31. (canceled)

32. (canceled)

33. (canceled)

34. (canceled)

35. An antibody which immunospecifically binds the complex according to claim 20 or a fragment or derivative of said antibody containing the binding domain thereof.

36. The antibody according to claim 35 which does not immunospecifically bind Mam, Mip1, Mip30 or Mip6 that is not part of a Mam:Mip1, Mam:Mip30 or Mam:Mip6 complex, respectively.

37. An isolated nucleic acid or an isolated combination of nucleic acids comprising (a) a nucleotide sequence encoding Mam and a nucleotide sequence encoding Mip1, (b) a nucleotide sequence encoding Mam and a nucleotide sequence encoding Mip30, (c) a nucleotide sequence encoding Mam and a nucleotide sequence encoding Mip6, (d) a nucleotide sequence encoding Mip30, or (e) a nucleotide sequence encoding Mip6.

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. A method of diagnosing or screening for the presence of or a predisposition for developing a disease or disorder characterized by an aberrant level of a complex of Mam and Mip1, Mam and Mip30 or Mam and Mip6, in a subject comprising measuring the level of said complex, RNA encoding Mam and Mip1, Mam and Mip30 or Mam and Mip6, or functional activity of said complex in a sample derived from the subject, in which an increase or decrease in the level of said complex, said RNA encoding Mam and Mip1, Mam and Mip30 or Mam and Mip6, or functional activity of said complex in the sample, relative to the level of said complex, said RNA encoding Mam and Mip1, Mam and Mip30 or Mam and Mip6 or functional activity of said complex found in an analogous sample not having the disease or disorder or a predisposition for developing the disease or disorder, indicates the presence of the disease or disorder or a predisposition for developing the disease or disorder.

57. (canceled)

58. (canceled)

59. (canceled)

60. (canceled)

61. (canceled)

62. (canceled)

63. (canceled)

64. (canceled)

65. A method of treating or preventing a disease or disorder involving an aberrant level of Mip1, Mip30 or Mip6 in a subject comprising administering to a subject in which such treatment or prevention is desired a therapeutically effective amount of a molecule that modulates the function of Mip1, Mip30 or Mip6, respectively.

66. (canceled)

67. (canceled)

68. (canceled)

69. (canceled)

70. (canceled)

71. (canceled)

72. A method of treating or preventing a disease or disorder involving an aberrant level of Mam in a subject comprising administering to a subject in which such treatment or prevention is desired a therapeutically effective amount of a molecule that modulates the function of Mam.

73. (canceled)

74. (canceled)

75. (canceled)

76. (canceled)

77. (canceled)

78. (canceled)

79. A recombinant non-human animal in which both an endogenous Mam gene and an endogenous Mip1 have been deleted or inactivated by recombination or insertional mutagenesis of said animal or an ancestor thereof.

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. A method of monitoring the efficacy of a treatment of a disease or disorder characterized by an aberrant level of a complex of Mam and Mip1 in a subject administered said treatment for said disease or disorder comprising measuring the level of said complex, RNA encoding Mam and Mip1, or functional activity of said complex in a sample derived from said subject wherein said sample is taken from said subject after the administration of said treatment and compared to (a) said level in a sample taken from said subject prior to the administration of the treatment or (b) a standard level associated with the pretreatment stage of the disease or disorder, in which the change, or lack of change in the level of said complex, said RNA encoding Mam and Mip1, or functional activity of said complex in said sample taken after the administration of said treatment relative to the level of said complex, said RNA encoding Mam and Mip1 or functional activity of said complex in said sample taken before the administration of said treatment or to said standard level indicates whether said administration is effective for treating said disease or disorder.

109. (canceled)

110. (canceled)

111. A purified protein selected from the group consisting of Mip30 and Mip6.

112. (canceled)

113. (canceled)

114. (canceled)

115. (canceled)

116. A purified fragment of a Mip30 protein comprising a domain of the protein selected from the group consisting of the C2H2-type zinc finger domain, the HMG-1 and HMG-Y DNA-binding domain (A+T-hook), and the bipartite nuclear localization signal.

117. (canceled)

118. (canceled)

119. (canceled)

120. (canceled)

121. A chimeric protein comprising a fragment of a Mip6 protein consisting of at least 20 amino acids fused via a covalent bond to an amino acid sequence of a second protein, in which the second protein is not the Mip6 protein.

122. (canceled)

123. (canceled)

124. (canceled)

125. (canceled)

126. An antibody which is capable of binding the Mip30 protein of claim 111.

127. An antibody which is capable of binding the Mip6 protein of claim 111.

128. (canceled)

129. (canceled)

130. (canceled)

131. (canceled)

132. (canceled)

133. (canceled)

134. (canceled)

135. (canceled)

136. (canceled)

137. (canceled)

138. (canceled)

139. (canceled)

140. A method of treating or preventing a disease or disorder in a subject comprising administering to a subject in which such treatment or prevention is desired a therapeutically effective amount of a Mip30 or Mip6 protein or derivative thereof which is able to bind to a Mam protein.

141. (canceled)

142. (canceled)

143. A method of treating or preventing a disease or disorder in a subject comprising administering to a subject in which such treatment or prevention is desired a therapeutically effective amount of a molecule, in which the molecule is an oligonucleotide which (a) consists of at least six nucleotides; (b) comprises a sequence complementary to at least a portion of an RNA transcript of a Mip30 or a Mip6 gene; and (c) is specifically hybridizable to the RNA transcript.

144. (canceled)

145. An isolated oligonucleotide consisting of at least six nucleotides, and comprising a sequence complementary to at least a portion of an RNA transcript of a Mip30 or Mip6 gene, which oligonucleotide is specifically hybridizable to the RNA transcript.

146. (canceled)

147. (canceled)


SEQUENCE LISTING (21 sequences)

SEQ ID NO: 1 - DNA, 6333 bp, Drosophila melanogaster
aaaacaacaaaaaaacagcggcagtcggacgcggtttttatcatcgagcgcggtttctttgttttagcgctcgaaaattgtcgagaactctcgtaaaaaatacactgtaccaaataactaaggagtgcatgctaattgaacaagcaaatgaattaaggtgtatatcaaagtgaaaagaagtcaaacataaccgattttgttggcggctttcttttatttttgttgagataccgtgtttaatttctccagttttatgcaaacacatgaataacataacagtggaaatttctaacagtgcattgtgagataaaattggattaaaattcaaagcattggataaatgctaagttgaaataacaacagaaataacaaatggaaaatagcaaatagcaaatcaaaggaaatcaaaatcaagttggattatcagcacagcttctgggacttgccacagaatgtgcacttggttttcttgtggatgctttcacctaccgcagcagcgatattcctaacacctctccgcgtagaataggcatttccggtcgtgcagtgcggaaacagcagcagcagcagcagcgctgctagcaacatcgggcgatatcagatatagacaacctatatacacatatatatttattcgccgccgctgtcgccgccttgttcttcgcgatttcgtcgccgtcagcgttcgccgccgactgagacgaaagctgcgaagcaacgagcgctagcgaaagcctcaccacggacgcatttatggatgcgggcggcctaccagttttccagagcgccagccaagcagccgccgtcgcccagcagcagcaacagcagcagcagcaacaacatttgaatctccagttgcatcagcaacacttgggcctccatctgcagcagcaacagcagctgcaactgcaacagcagcagcacaatgcccaggcgcagcagcagcagatccaagttcagcagcagcagcagcagcagcagcaacaacagcagcagcagcagcaacactcgccctacaatgcaaacctgggagcaacaggcggcattgctggaatcactggcggcaacggagctggcggccctacaaatccaggagcggtacccaccgctccgggcgacactatgcccaccaagcggatgccggtcgtcgatcggctgcgacgccgcatggagaactaccgccgcaggcagacggattgcgtgccacgctatgagcaggccttcaataccgtctgcgagcagcagaaccaggagaccaccgtcctccagaagcgcttcctcgagagcaagaacaagcgggcggccaagaagaccgacaagaagctgcccgatccctcgcagcagcatcagcagcagcagcaccaacagcagcagcagcatcagcagcaccagcaacaccagcaggcccaaactatgttagccggacagctgcagagcagtgttcatgtgcaacaaaaattcctgaaacgacccgcggaagatgttgacaatggccctgacagctttgaaccgccgcacaagttgcctaataataacaacaatagcaatagcaacaacaacaatggcaatgcgaatgccaacaacggcggcaatggctccaataccggcaacaacacaaacaacaatggcaacagcaccaacaacaacggcggcagcaacaacaacggctctgagaatctaaccaaattctcggtggaaattgttcagcaactggagtttaccacatcgccggccaattcgcaaccgcaacagattagcactaatgtcactgtaaaggcgctgaccaacacatcggtgaaaagtgaaccaggtgtgggcggtggcggagggggcggcggtggaggcaatagcggtaacaacaacaacaacggaggcgggggaggcggcggaaacggtaataacaacaacaacggaggtgatcatcaccagcaacagcaacagcaccagcaccagcagcagcagcagcaacaaggcggcggactgggtggcctcgggaataatggaaggggcgggggacccggcggcatggcgacgggtcccgggggcgtggccggtggacttggcggcatgggcatgccacccaacatgatgtccgcccaacagaagtcggccctcggcaatctggccaatttggtggagtgcaaacgggaaccggatcacgattttcccgatctcggctcattggacaaagatggcggtggcggacagtttcccggctttcccgatctactcggggatgataactcggagaacaacgacacgttcaaggatctcatcaacaacctgcaggacttcaatcccagtttcctcgatggcttcgatgagaaaccgctgctggacataaagacggaggatggcatcaaggtggagccgcccaatgcccaggatttgatcaatagcctgaatgtgaagtccgagggcggtttgggtcatggtttcggtggctttggcctgggtctggacaatccgggcatgaagatgcgtggcggcaatcctggcaaccaaggtggctttcccaacggtccgaatggcgggacaggtggtgctcccaatgctggtggtaatggtggaaactctggtaatctcatgtccgaacatccgttggctgctcagacactcaagcaaatggccgagcagcatcagcacaagaatgccatgggtggcatgggtggattcccacgaccgccgcacggcatgaatccgcagcaacagcagcagcagcaacaacagcagcaacagcaacaggcccagcagcaacatggtcaaatgatgggacaaggacagccgggtcgctataacgactacggcggcggctttcccaatgactttggcctgggacccaatggtccgcagcagcagcagcagcaggcgcagcaacagcagccgcagcagcaacacctgccgccgcagttccatcagcaaaagggtcctggcccaggagccggcatgaatgtgcaacagaatttcctggacatcaagcaggagctcttctattcctctcaaaacgatttcgatctcaagcgtctgcagcagcagcaggccatgcaacagcagcagcagcagcaacaccatcagcagcagcagcccaaaatgggtggtgttcccaatttcaacaaacaacagcagcagcaacaggtgccgcagcagcaactgcagcagcagcagcagcagcagcaacaacagcaacagcagcagcagcagtactcacccttcagcaaccagaaccccaatgcggcggccaacttcctcaattgtccgccaaggggcggtccaaatggcaaccagcagccgggcaatctggcgcagcagcaacagcaacccggggctggaccgcagcagcaacagcagcgcggcaacgccggcaatgggcagcagaacaatccgaataccggacccggcggcaacaccccaaatgcgccgcagcagcagcagcaacagcaatcgaccaccacaacgctgcagatgaagcagacgcagcagttgcacattagccagcagggtggcggtgctcaaggtattcaggtgtcggctggtcagcatctgcatttgagtggcgacatgaagagcaacgtctcggtggccgcccagcaaggtgtcttcttcagtcagcagcaggcgcaacagcaacagcagcagcaacagcctggcggcaccaacggaccgaatccccagcagcagcagcaacagccgcatggcggcaacgctggcggtggagtaggcgttggcgtgggcgtgggtgtgggcaacggaggtcctaatcccggacagcagcagcagcaaccaaatcaaaacatgagcaatgcaaatgttccctccgatggcttctcgctctcccagagccaaagcatgaactttaaccagcagcagcagcaacaggcggccgcccagcagcagcaggtgcagcccaatatgcgccagcgtcagacgcaagcgcaggcagcggctgcagcagcggcagcagcggcgcaggcgcaggcggcagccaatgccagcggaccgaatgtgccgctcatgcagcagccgcaggttggagtgggcgtaggcgtcggcgtgggcgtgggcgtgggtgtgggcaatggtggcgtggtcggtggacccggttccggtggacccaacaacggtgcaatgaatcagatgggcggacccatgggcggcatgccgggcatgcagatgggtggacccatgaacccgatgcaaatgaaccccaacgctgccggtccaaccgcccagcagatgatgatgggcagcggcgctggcggaccgggtcaggttccgggacctggccaaggaccaaatccgaatcaagccaagttcctgcagcagcagcagatgatgcgcgcccaggcgatgcagcaacagcagcagcacatgtctggagcacgaccaccaccgcccgagtacaatgccaccaaggcgcagttgatgcaggcgcagatgatgcagcaaacggtgggcggcggtggtgtcggcgtcggaggtgtgggcgtaggcgtgggcgtgggaggggtcggtggtgccaacggtggccgctttccgaacagcgctgcccaggcggcggccatgcggcgcatgacccagcagcccataccgccatccggtccgatgatgcgaccacagcatgcgatgtacatgcagcaacatggaggagcaggcggtggcccaaggaccggtatgggtgtgccctatggtggcggacgaggcggtccaatgggcggtccgcagcagcagcagaggccgcctaatgtccaggttacgcccgatggcatgcccatgggttcgcagcaggagtggcggcacatgatgatgacacagcagcagacgcaaatgggcttcggtggaccaggaccaggtggacccatgcgccagggacccggtggatttaatggaggcaactttatgcccaatggtgcacccaatggtgcagcgggcagtggacccaacgctggcggcatgatgaccggccccaatgtgccgcaaatgcagctgaccccagcccagatgcaacagcaactgatgcgccaacagcagcagcaacagcaacagcagcagcagcagcacatgggacctggtgccgccaataacatgcagatgcagcaactgctgcagcagcagcaatccggaggcggcggcaacatgatggccagccagatgcagatgaccagcatgcacatgacccagacccagcagcagatcaccatgcagcagcagcagcagttcgtgcagagcaccaccacgaccacgcaccagcagcagcagatgatgcaaatgggtccagggggcggtggtggtggcggcgggccgggatcggccaacaacaacaatggtggcggcggtggtggagcagcgggcggaggcaactccgcatcgaccattgccagtgccagttccattagccagacgatcaactcggtggtggccaactcgaatgatttcggtctcgaattcttggacaacctgcccgtggacagtaacttctccacgcaggatctgatcaactccctcgacaacgacaacttcaatctgcaggacttcaatatgccgtagacgattgtcgataaccactcaaacaccacctccagccagcaccaaacataaccaccatcaccaccataaccaccacgatgttgcctcagttttgtttagattttttttattttgttgcctcttgttgttattggcctggacagccgaacccaaccgacccgacctgacgcccaaccgccccacacggccccgccccccttgttcctgccaacgaagttattttgtttattatatgtgtgtgaatttattcctaagctaagttcgttcgtctatgtactctaatttcgtgggctccactgtattttattgtattgtattgtatttgatcgtatttgtatgtattattagagctgttcccaaggtccaaggcgagcggccaatgggcgtggtagaggcgagagttcgcaaaagacattccatatgcaacggtcgaacgaaggaattaaagaaattcgagaaagtatttgcaagtactttgaaaacaattgaacagttttcagattctcatacaccgatacagatacagatactacagactcgttgtgcaacttaagcacttgtacttaatgcaaatttgtagtgccacgcccagcggattgcacgccccatttcgtttgattacttaagcaaaaaccagatgaaatgatggtagcaaacacaaaaagaaccaacaacaaaacaaaatcgaaaaacaaaacacaaaatagttgtacattttatattatatttaatttaatttaatttttataaaaacaaaatcgacaaactaagagaaacaaaaaaaaaaaa

SEQ ID NO: 2 - protein, 1596 aa, Drosophila melanogaster
MDAGGLPVFQSASQAAAVAQQQQQQQQQQHLNLQLHQQHLGLHLQQQQQLQLQQQQHNAQAQQQQIQVQQQQQQQQQQQQQQQQHSPYNANLGATGGIAGITGGNGAGGPTNPGAVPTAPGDTMPTKRMPVVDRLRRRMENYRRRQTDCVPRYEQAFNTVCEQQNQETTVLQKRFLESKNKRAAKKTDKKLPDPSQQHQQQQHQQQQQHQQHQQHQQAQTMLAGQLQSSVHVQQKFLKRPAEDVDNGPDSFEPPHKLPNNNNNSNSNNNNGNANANNGGNGSNTGNNTNNNGNSTNNNGGSNNNGSENLTKFSVEIVQQLEFTTSPANSQPQQISTNVTVKALTNTSVKSEPGVGGGGGGGGGGNSGNNNNNGGGGGGGNGNNNNNGGDHHQQQQQHQHQQQQQQQGGGLGGLGNNGRGGGPGGMATGPGGVAGGLGGMGMPPNMMSAQQKSALGNLANLVECKREPDHDFPDLGSLDKDGGGGQFPGFPDLLGDDNSENNDTFKDLINNLQDFNPSFLDGFDEKPLLDIKTEDGIKVEPPNAQDLINSLNVKSEGGLGHGFGGFGLGLDNPGMKMRGGNPGNQGGFPNGPNGGTGGAPNAGGNGGNSGNLMSEHPLAAQTLKQMAEQHQHKNAMGGMGGFPRPPHGMNPQQQQQQQQQQQQQQAQQQHGQMMGQGQPGRYNDYGGGFPNDFGLGPNGPQQQQQQAQQQQPQQQHLPPQFHQQKGPGPGAGMNVQQNFLDIKQELFYSSQNDFDLKRLQQQQAMQQQQQQQHHQQQQPKMGGVPNFNKQQQQQQVPQQQLQQQQQQQQQQQQQQQQYSPFSNQNPNAAANFLNCPPRGGPNGNQQPGNLAQQQQQPGAGPQQQQQRGNAGNGQQNNPNTGPGGNTPNAPQQQQQQQSTTTTLQMKQTQQLHISQQGGGAQGIQVSAGQHLHLSGDMKSNVSVAAQQGVFFSQQQAQQQQQQQQPGGTNGPNPQQQQQQPHGGNAGGGVGVGVGVGVGNGGPNPGQQQQQPNQNMSNANVPSDGFSLSQSQSMNFNQQQQQQAAAQQQQVQPNMRQRQTQAQAAAAAAAAAAQAQAAANASGPNVPLMQQPQVGVGVGVGVGVGVGVGNGGVVGGPGSGGPNNGAMNQMGGPMGGMPGMQMGGPMNPMQMNPNAAGPTAQQMMMGSGAGGPGQVPGPGQGPNPNQAKFLQQQQMMRAQAMQQQQQHMSGARPPPPEYNATKAQLMQAQMMQQTVGGGGVGVGGVGVGVGVGGVGGANGGRFPNSAAQAAAMRRMTQQPIPPSGPMMRPQHAMYMQQHGGAGGGPRTGMGVPYGGGRGGPMGGPQQQQRPPNVQVTPDGMPMGSQQEWRHMMMTQQQTQMGFGGPGPGGPMRQGPGGFNGGNFMPNGAPNGAAGSGPNAGGMMTGPNVPQMQLTPAQMQQQLMRQQQQQQQQQQQQHMGPGAANNMQMQQLLQQQQSGGGGNMMASQMQMTSMHMTQTQQQITMQQQQQFVQSTTTTTHQQQQMMQMGPGGGGGGGGPGSANNNNGGGGGGAAGGGNSASTIASASSISQTINSVVANSNDFGLEFLDNLPVDSNFSTQDLINSLDNDNFNLQDFNMP

SEQ ID NO: 3 - DNA, 5718 bp, homo sapiens
cggccgcggcggtagcgcggaaaacaatggggccggggcggtggggagaggccgaggcttgaggtaggcagcaagcgccggctgggggtcgggccgagcggggcaggaggaaaacccgccgccgcgcgcgagcccgctccgctgccctcgggggcatggcgcggccgtgaggcggagaggggtagccgcggggagcgaagcccgcagtgccagccggccccgagaggcccggccccgggcccggcccgtgcagcccgcggcccatggtgctgcccacctgccccatggcggagttcgcgctgccgcggcacagcgcggtcatggagcgccttcgccggcgcatcgagctgtgccggcgccaccacagcacctgcgaggcccgctacgaggccgtgtcgcccgagcgcctggagctggagcgccaacacaccttcgccctgcaccagcgctgcatccaggccaaggccaagcgcgccgggaagcacaggcagccgcccgccgccacggccccggcgcccgccgccccggccccgcgcctggacgccgctgacggccccgagcacggccgcccggccacgcatcttcatgatacagttaagaggaatcttgacagcgccacttcccctcagaatggcgatcaacagaatggctacggggacctctttcctgggcataagaagactcgccgggaggcccctctgggagttgccatctcttccaatggactgcctccagcctcccccctcggtcagtctgacaagccttctggagccgacgccctgcagtccagtgggaagcactctctggggctagactctctcaacaaaaagcgtctggctgactccagccttcacttgaatggaggcagtaaccccagtgagtcatttcctctgagcctgaataaagaactgaagcaggagcctgtcgaagacctgccttgcatgatcactgggactgtcggctccatatcgcaaagcaacctcatgccagacctcaaccttaacgagcaggagtggaaggagctcatcgaggagctgaacaggtcggtgcccgatgaagacatgaaggacctgtttaatgaggacttcgaggagaagaaggacccagagtcttctggctctgccacacaaacccccttggcacaggacattaatattaagacggaattctctccagcagcctttgagcaagaacagttaggctctccacaagtgagggccgggtctgcagggcagacctttctggggccttcctctgcccctgtgagtacagattcccccagcctagggggctcccaaaccttattccacacctctggtcagccccgggcggacaatcccagtccaaacctgatgccggcatcagcccaggcccagaacgcacaaagagcccttgcaggtgtggtattgcccagtcagggcccaggaggggcctcagagctgtcctctgcccaccagctccagcagatcgctgccaagcagaagcgcgagcagatgctccagaacccacagcaggccaccccggcaccagccccgggccagatgtccacatggcagcagacggggccctcccacagttccttagatgtcccttaccccatggagaagcctgccagcccttccagctacaagcaagacttcactaactccaaactgctcatgatgcctagtgtgaataagagttcccctcggcccggaggcccctacctccagcccagccatgtgaacctgctgagtcaccagccaccgagtaacttgaatcagaactccgcgaataaccaggggtctgtgctggactacggcaatacaaaacccctttctcattacaaagcggactgtgggcaaggcagcccggggtctggccagagcaagccagccctgatggcttatcttccccagcagctgtcccatataagtcacgagcagaactccctgtttctgatgaagccaaagccaggaaatatgcctttccgatcactggttccacctggccaggagcagaacccttccagtgtccctgtgcaagcccaggctaccagtgttgggacccagccgcctgccgtgtccgtggccagctcccacaacagctccccctatctcagcagccagcaacaggccgctgtaatgaagcagcatcagttgcttttggaccaacagaaacaaagggagcagcagcaaaagcatttacagcaacagcagttccttcagaggcaacagcaccttctcgcggaacaggagaagcaacagtttcagcgccatctgacccgcccaccaccccagtaccaagacccgacacaaggcagcttcccacagcaggttggacagttcacagggtcctctgctgccgtgcccggcatgaacaccttgggtccatccaactccagctgtcctcgagtgttccctcaggctgggaatctgatgccaatgggccctggacatgcttcagtttcctctctccccacaaactcaggccaacaggaccggggtgtggctcagttccctggctcccaaaacatgcctcagagcagcctctatggcatggcttctggcataacccagatagttgcccagcccccgccacaggccaccaatggacatgcccacattccacggcagaccaacgtgggccagaacacctccgtctcagctgcctatgggcagaactctctgggaagctctggcctctcccagcagcacaataaggggaccctgaaccctggtttaacaaagccaccggtcccaagggtgtcaccagccatgggaggccagaattcctcctggcagcatcagggaatgccgaacctcagtggccagaccccagggaacagcaacgtgagtcccttcactgcagcctccagtttccacatgcagcagcaggcccacctgaaaatgtctagcccgcaattctcccaggcagtgcccaacaggcccatggctcccatgagctcagcagctgccgtggggtccttgctacccccagtgagtgcacagcagaggaccagcgcccctgccccagcaccacccccaacagcccctcagcagggcttgcctggcctgagcccagcagggcctgagctgggggccttcagccagagccctgcctcacagatgggcggtcgggcggggctgcactgcacccaggcctaccctgtgcggaccgcgggccaggagctgccttttgcctatagcgggcagccaggtggcagtgggctctctagtgtggctggacacaccgatctgatcgactccctgctgaagaacaggacttcagaggagtggatgagtgatttggacgacctgttagggtctcagtaatggaaggatttgtagtgtttttagtgttcattcatcctatatttttattctcagattcaaagaaagagcaactactttggaccaaaagcccatggcctggggagctgggcaggtagagcccaagctccaggtgaggcctggccctgggcagggtctgtggctgcgcccctcaggccagcagttgaggtccatcgggctggccccagcccatctgctggcatcagtacctggtgttgggacagcaggatagggttctaaaggtggttttctatccaaacgaccaaaaaaccaacagtaacaccagtgaaaccccacactgtcgggcttataaaaatctgtgccatcatggtgattttatccaagactgctccacttaccccagtgctggggacaagtttctgttgaaactttagatagcagaattatttgcaatttgtagcatagaaaagattttttaaatttttttacaaaaggtttttaaacagattagggtaggtgatggtttaaatcaattaagtggcattggaaacctagggtttccttttgattaagagccttttttgtttctgctctttgtcagctttcaggggagaaggaggccactggaaaattatttccctaagtgcaggctgttgactgcgtatgccaaaaagggacaggaggcatgggatagcaggtctggtgacacagctagggtcttcctagcagctcctcctcctccctcccaaggcccccaggaatcccttcctcccatgtcctggcagcaggaccccaggctacatatggaaggtagagatgtgggggtcctgtatcctggagtattatgtctccccaccttctgcagttttctctgaacatgtatgttgcccatggtgggagcgtggtcactgtgcagttgtgcacagatgtctttcctttaccgttggcctttctgtctgcctctccttcctctctgcagcccaaatggaaaacaattatttactccattggagggaaaggaagagtcttagaattcctaagggaaccttagcataaaggttttggggaaggaggccgtaggccggcccggaggaagcaattccacttggtttgacaacttctgccactcccatgtcagatgacttgcacttcttaaagagattgctttataacactaagacatcctttctaaagattcaagtggacttgactaagctgagggtccacgaaatagaatatgacatgtgagctgtttttggaaaacgaagatggagagagcacttccccgtaacgaaagcaaagtggtaagcacagggtgagacccttttacacagaatggtggagagaaaagagaatgctgaaaagtggctcagatgcagagtgttctgtggagaaactgcagccccacttctgtttccctggagtctcccaatggatcattcaggagtgtcctatgtgagaattgagccaaggaaaatactcatgcaaccagcctgagtcgcggtgaggggacgagaggttgtacacacattggtagttattttgcaccagcagtgcctttctcactgggggtacttggaccctcagatcttcttttctaatagccatttgccaccccaagtggtatgtcggccatttctccttaaaacaccttccctacctttcccatgtactcagtttagctctcaaagaaggggtgaatcataaagccagtgaaaatttcaccctctgagggagttccccaatctgaaggggaagagggtgacctcagcggcttttctcccaaaaatcggctgaaggctggttgtggatccttgttcctctcctgaccccatctggctgctgccccgtctcccacccctgtccccggggctcgctggccctgcactccgccttagtcctggggccggcgacacagtgggggctcctcacttgctgcagtgtcatagcaataaaatgtgattcttggggtccccccagggagctgcccatggctttatttatgaacctggttttcgggagtcaggggaggagatgactttgcttctgtgcacagccccgtcttccaggagccacaactcagaagaaaagggtgctcagacttttgttatacacatttgctttgtgtaaataaatgtttacaattttatatgaaagatggaataagcgctagagcttccaactgtatattttttacttttatagattttaaaactatgatcctttatatgtgtgttttgggggagctatgataagttttatggcaaacggttggtattgttaactttttattgtcatcaaaagttcataaaagtcctattaatccccatattcttctactgcccttaactctggtatacaccaaaaagaaatctttactttccttgttttatcattataaaaataaagtattttgctagtatgg

SEQ ID NO: 4 - protein, 1016 aa, homo sapiens
MVLPTCPMAEFALPRHSAVMERLRRRIELCRRHHSTCEARYEAVSPERLELERQHTFALHQRCIQAKAKRAGKHRQPPAATAPAPAAPAPRLDAADGPEHGRPATHLHDTVKRNLDSATSPQNGDQQNGYGDLFPGHKKTRREAPLGVAISSNGLPPASPLGQSDKPSGADALQSSGKHSLGLDSLNKKRLADSSLHLNGGSNPSESFPLSLNKELKQEPVEDLPCMITGTVGSISQSNLMPDLNLNEQEWKELIEELNRSVPDEDMKDLFNEDFEEKKDPESSGSATQTPLAQDINIKTEFSPAAFEQEQLGSPQVRAGSAGQTFLGPSSAPVSTDSPSLGGSQTLFHTSGQPRADNPSPNLMPASAQAQNAQRALAGVVLPSQGPGGASELSSAHQLQQIAAKQKREQMLQNPQQATPAPAPGQMSTWQQTGPSHSSLDVPYPMEKPASPSSYKQDFTNSKLLMMPSVNKSSPRPGGPYLQPSHVNLLSHQPPSNLNQNSANNQGSVLDYGNTKPLSHYKADCGQGSPGSGQSKPALMAYLPQQLSHISHEQNSLFLMKPKPGNMPFRSLVPPGQEQNPSSVPVQAQATSVGTQPPAVSVASSHNSSPYLSSQQQAAVMKQHQLLLDQQKQREQQQKHLQQQQFLQRQQHLLAEQEKQQFQRHLTRPPPQYQDPTQGSFPQQVGQFTGSSAAVPGMNTLGPSNSSCPRVFPQAGNLMPMGPGHASVSSLPTNSGQQDRGVAQFPGSQNMPQSSLYGMASGITQIVAQPPPQATNGHAHIPRQTNVGQNTSVSAAYGQNSLGSSGLSQQHNKGTLNPGLTKPPVPRVSPAMGGQNSSWQHQGMPNLSGQTPGNSNVSPFTAASSFHMQQQAHLKMSSPQFSQAVPNRPMAPMSSAAAVGSLLPPVSAQQRTSAPAPAPPPTAPQQGLPGLSPAGPELGAFSQSPASQMGGRAGLHCTQAYPVRTAGQELPFAYSGQPGGSGLSSVAGHTDLIDSLLKNRTSEEWMSDLDDLLGSQ

SEQ ID NO: 5 - DNA, 4989 bp, Homo sapiens
agcagcggcggcagcggcagcccagccgagcgttaggtgctgctctctgcgcggcgttttgcaaaggacttcaccgatctacttttgcagtcgcctcggactgtccatgtgtttacttcccccagcccgaggattcgatatctaggttcctgtgaaatgcaactgagcagccaaagtactttgagaacacggggcggcataaacaccaaaacttttttgtggaaggaaaatgcaataagcaagcttgccgttttccgatgcggtgtggagtgagtgtgtgtcgcgcgtgtccgcactggaggcatatgcttgtgtgtgtacatggggtgtgtttttcggtatgtagggagaaaatgcttgccaaccaccggaaatctcctggaatttattagaaaataatggattataaaaagaaggcaagcaaggagcggatctccccttgagttgcaacccgatttgctgctggctcagtttgttgtgattctttttgttgataggtgtctgatggtattccgataacgttccccccttttcttccccttgagcttttacagtttaaaaaaaggaaacaaaaaccaccccaaaatctccccccccgtttttttcgccccgtcgggatcgccgtttccatccatgtgcttgcgtctcccccgcgttccacttaaactattttaatccttggacccaaggaggaggctgataggggggtggataaaaaaagttcttccaaaatagtgtgcccggggagcaggatgggggatttcgcagcccccgctgctgccgcgaatggcagtagtatttgcatcaacagtagcctgaacagcagcctcggcggggccgggatcggtgtgaataatactcccaatagtactcccgctgctccgagtagcaatcacccggcagccggtggatgcggcggctccgggggccccggcggcggttcggcggccgttcccaagcacagcaccgtggtggagcggctccgccagcgcatcgagggctgccgtcggcaccacgtcaactgcgagaacaggtaccagcaggctcaggtggagcagctggagctggagcgccgggacaccgtgagcctctaccagcggaccctggagcagagggccaagaaatcgggcgccggcaccggcaaacagcagcacccgagcaaaccccagcaagatgcggaggctgcctcggcggagcagaggaaccacacgctgatcatgctacaagagactgtgaaaaggaagttggaaggagctcgatcaccacttaatggagaccagcagaatggtgcttgtgatgggaatttttctccgactagcaaacgaattcgaaaggacatttctgcggggatggaagccatcaacaatttgcccagtaacatgccactgccttcagcttctcctcttcaccaacttgacctgaaaccttctttgcccttgcagaacagtggaactcacactcctgggcttctagaagatctaagtaagaatggtaggctccctgagattaaacttcctgtcaacggttgcagtgacctggaggatagcttcaccatcttgcagagcaaagacctcaaacaagaacctctcgatgaccctacttgcatagacacatcagaaacatctctttcaaatcagaacaagctgttctcagacattaatctgaatgatcaggagtggcaagaattaatagatgaattggccaacacggttcctgaggatgacatacaggacctgttcaacgaagactttgaagagaagaaggagccagaattctcgcagccagcaactgagacccctctctcccaggagagtgcgagcgtgaagagcgacccctctcactctcccttcgcacatgtctccatgggatctccccaggcgaggccttcttcttctggtcctcccttttctactgtctccacggccactagtttaccttctgttgccagcactcccgcagctccaaaccctgcaagctcaccagcaaactgtgctgtccagtcccctcaaactccaaaccaagcccacactccaggccaagctccacctcggcctggaaatggttatctcctgaatccggcagcagtgacagtggccggttcagcgtcagggcctgtggctgtgcccagctctgacatgtctccagcagaacagctcaaacagatggctgcacagcagcaacaaagggccaaactcatgcagcagaaacagcaacagcaacagcagcagcagcagcagcagcagcaacagcagcagcagcagcagcaacagcactcaaatcagacttcaaattggtctcccttaggacctccctctagtccatatggagcagcttttactgcagaaaaaccaaatagcccaatgatgtacccccaagcctttaacaaccaaaaccctatagtgcctccaatggcaaacaacctgcagaagacaacaatgaataactacctccctcagaatcacatgaatatgatcaatcagcagccaaataacttgggtacaaactccttaaacaaacagcacaatattctgacttatggcaacactaaacccctgacccacttcaatgcagacctgagtcagaggatgacaccaccagtggccaaccccaacaaaaaccccttgatgccgtatatccagcagcagcaacagcagcagcaacagcaacagcagcagcagcagcagcagcagccgccacctccacagctccaggcccccagggcacacctgagcgaagaccagaaacgcctgcttctcatgaagcagaaaggagtgatgaatcagcccatggcttacgctgcacttccatcccacggtcaggagcagcatccagttggacttccccgaaccacaggccccatgcagtcctccgtgcccccaggctcaggtggcatggtctcaggagccagtcccgcaggccccggcttcctgggcagccagccccaagcagccatcatgaagcagatgctcattgatcagcgggcccagttgatagagcagcagaagcaacagttcctgcgggagcaaaggcagcagcagcagcagcagcagcagattttggcggaacagcagttgcagcaatcacatctaccccggcagcacctccagccacagcggaatccatacccagtgcagcaggtcaatcagtttcaaggttctccccaggatatagcagccgtaagaagccaagcagccctccagagcatgcgaacgtcacggctgatggcacagaacgcaggcatgatgggaataggaccctcccagaaccctgggacgatggccaccgcagctgcgcagtcggagatgggactggccccttatagcaccacgcctaccagccaaccaggaatgtacaatatgagcacaggcatgacccaaatgttgcagcatccaaaccaaagtggcatgagcatcacacataaccaagcccagggaccgaggcaacctgcctctgggcagggggttggaatggtgagtggctttggtcagagcatgctggtgaactcagccattacccagcaacatccacagatgaaagggccagtaggccaggccttgcctaggccccaagcccctccaaggctgcagagccttatgggaacagtccagcaaggagcacaaagctggcaacagaggagcttgcagggcatgcctgggaggactagtggagaattgggaccattcaacaatggcgccagctaccctcttcaagctgggcagccgagactgaccaagcagcacttcccacagggactgagccagtcagtcgtggatgctaacacgggcacagtgaggaccctcaacccagctgccatgggtcggcagatgatgccatcgctcccggggcagcaaggcaccagccaggcgaggccaatggtcatgtctggcctgagccagggagtcccaggcatgccagcgttcagccagcccccagcacagcagcagatacccagtggcagctttgctccaagcagccagagccaagcctatgagcggaatgcccctcaggacgtgtcatacaattacagtggcgacggagctgggggttccttccctggcctcccggacggtgcagaccttgtggactccatcatcaaaggcgggccaggggacgagtggatgcaggagcttgatgaattgtttggtaacccctaatcaagagaggccccaagatccacaactcgagtggttaaagcttaaaaagtgaaaaagaaacaggatgttgacccatccttgttttttgtttttttgacccacgtaaactgagcaaaactgcagctggctgacaatggaagatccaggtgccaatccacagccccaccaggcctcatttcacctgattttcacacagcaatcgagatgagacgccatgcagatcccggctgcgagagagggagacacccggaggagcaggtgggaagatgaagccggccagagcccctctgcccagcatgccctgtgatcgcctggcccagcaggagctgcttcagccgagagggactattacccaagagaggtatcctcagcccctcctgccccaggtcgggagacagcagctttggagacacaaaagagacagagcctcagccagggagagtgagtcccccagaagaggctgggtggttgcacaggccaggtgcacaggttggaaatgcactgaactctgggtgccgagagatgtaaggctttgagacatgctactgaatttggagggcaggcacgaagaacagtgagattgtcaaaaggagacaaccacagatcctacaggactgtctgtctcctgccccatgatgaccctcaggaattgcaaaggctctgctgtcacaaggagagcaggctgagtttggagcagggtccatccggcagtcctgggacggcttccctctgctggtgcccctggtggcagtccctccaggtggggctggagcctgctggcgcccaatacaaaacccatacatccaggtgggtcacatctacttctggcggccgc

SEQ ID NO: 6 - protein, 1133 aa, homo sapiens
MGDFAAPAAAANGSSICINSSLNSSLGGAGIGVNNTPNSTPAAPSSNHPAAGGCGGSGGPGGGSAAVPKHSTVVERLRQRIEGCRRHHVNCENRYQQAQVEQLELERRDTVSLYQRTLEQRAKKSGAGTGKQQHPSKPQQDAEAASAEQRNHTLIMLQETVKRKLEGARSPLNGDQQNGACDGNFSPTSKRIRKDISAGMEAINNLPSNMPLPSASPLHQLDLKPSLPLQNSGTHTPGLLEDLSKNGRLPEIKLPVNGCSDLEDSFTILQSKDLKQEPLDDPTCIDTSETSLSNQNKLFSDINLNDQEWQELIDELANTVPEDDIQDLFNEDFEEKKEPEFSQPATETPLSQESASVKSDPSHSPFAHVSMGSPQARPSSSGPPFSTVSTATSLPSVASTPAAPNPASSPANCAVQSPQTPNQAHTPGQAPPRPGNGYLLNPAAVTVAGSASGPVAVPSSDMSPAEQLKQMAAQQQQRAKLMQQKQQQQQQQQQQQQQQQQQQQQQHSNQTSNWSPLGPPSSPYGAAFTAEKPNSPMMYPQAFNNQNPIVPPMANNLQKTTMNNYLPQNHMNMINQQPNNLGTNSLNKQHNILTYGNTKPLTHFNADLSQRMTPPVANPNKNPLMPYIQQQQQQQQQQQQQQQQQQPPPPQLQAPRAHLSEDQKRLLLMKQKGVMNQPMAYAALPSHGQEQHPVGLPRTTGPMQSSVPPGSGGMVSGASPAGPGFLGSQPQAAIMKQMLIDQRAQLIEQQKQQFLREQRQQQQQQQQILAEQQLQQSHLPRQHLQPQRNPYPVQQVNQFQGSPQDIAAVRSQAALQSMRTSRLMAQNAGMMGIGPSQNPGTMATAAAQSEMGLAPYSTTPTSQPGMYNMSTGMTQMLQHPNQSGMSITHNQAQGPRQPASGQGVGMVSGFGQSMLVNSAITQQHPQMKGPVGQALPRPQAPPRLQSLMGTVQQGAQSWQQRSLQGMPGRTSGELGPFNNGASYPLQAGQPRLTKQHFPQGLSQSVVDANTGTVRTLNPAAMGRQMMPSLPGQQGTSQARPMVMSGLSQGVPGMPAFSQHPAQQQIPSGSFAPSSQSQAYERNAPQDVSYNYSGDGAGGSFPGLPDGADLVDSIIKGGPGDEWMQELDELFGNP

SEQ ID NO: 7 - protein, 792 aa, artificial sequence
  consensus sequence
MDFAAAAANGIVNNLGGGIGNPTPAPACPGAAPRHSVVERLRRRIECRRHHTCERYQAVEQLELERQTVLQRLEQAKKRATGKQQHQPPQQDAAEHRHTLHQTVKRLDGASPLNGDQQNGGNFPGTRRAGAINNSNGLPLPASPLQDLKPSGDLQSGHGLDLNGRLPALPVNGGSPESFPLQSLNKLKQEPDDPCIDTTGSLSQNLFPDLNLNDQEWELIDELNVPDDIQDLFNEDFEEKKPESGATETPLAQDINKSHFSPFLGSPQRPGSGFLPSSANPSSPNGGSQTHTGQAPANPSPGNLMPAAQAQNAGAGPVVPSPGAELMAQQQQAKQQKQQQQQQQQAQQQGQMQQPHSNSDPYGPPSPYDFTPNSMMPQANQPPPQYLPHNQPPNNQNSLNKQLYGNTKPLHADQPGGVPNKPLMYQQQQQQQQQQQQQQQQQPQQSPKSEQNLFLMKPKPGKQPLPGQEQPVGPQRATGQPPSGVASPNAPLSQQQAAMKQHIQAQLIQQKQQFREQQQQQQQQQQLAEQQQQPRHLPNPPQQNPFQGSFPQAAQVQMRTSAQNAVPGMGGPSNPPGMQMGPSPTNSGQQGAQPGQNMPQYMSGMTQMQPPPNQGHQAQPRQTVGQGVGVSAGGQNSMLSSGQQHPMKGPGLPPQPPRVSPMGQGQSWQQQGMPGTGPGPFNGASQGQQNFMPQGSQAVPNGRAPAAGMMPPQQTSAPAMQQGLQGPGMPLGAFSQHPAQQQIGFHQQAYPRAGPNYSGGAGGSGLSSVAGDLVDSKNTEWMQDLDDLFGMP

SEQ ID NO: 8 - DNA, 2441 bp, Drosophila melanogaster
ctttatacgtttcccaacacctgcaccggcggcgtcacttttctcggcttttccgtatcgcaattttgcaattttctcgccaaaaataagaaaaaggaaaacgtaaattcagaaaatggcagcagctatcaatggtgttttcccacccacattgcaggagctggtaaagaagtccaaggtgctggtcgtgggcgccggcggaatcggctgcgaggtgctcaaaaaccttgtgcttagcggctttacagacatcgaaattatcgatctggacacaattgatcttagcaacttgaaccgacagttcctattccatcgcgagcacgttggaaaatctaaggcacgggtggctagggaaagcgcactgagcttcaatccggacgctaagataaccgcttaccacgacagtgtaacatctactgattatggcgtcaacttctttaagaagttcgatttggtgctcagcgccttggacaacagggccgccaggaatcatgtaaatcgcatgtgcctcaatgcggatgtcccgctgatcgagagcggcaccgccggctacaatggccaggtggaactgatcaagcgtggactcacccagtgctacgagtgcactccaaaagacaaacagcgaagcttcccgggctgcaccatacgcaatacgccctccgaaccgattcactgcattgtctgggccaagcatctctttaatcaactgtttggtgagtccttggaagatgaggatatttctcctgatgctgctgatcccgatgcaaaagaaaaagacggcggcgatggaaatggcgagcccaaaggcgatgggaaggaaaaaggcgaagagtcaaaagaggaaaaggaggccaaggaggatactgccaacggcaacataatgcgcattaatactcgccaatgggccaaggactgcaactatgatgcgggcaagctgttcaacaagttctttaacgaggacattacctatttgttgcgtatgtcgaatttgtggaagacccgcaaggcacccgtgcccgtgcagtgggataccctgctgcccgaaggatcctccggtgatcagaaggatgtggccaagcagcatcacaaggtgtggtccatcgaggagtgcgctcaggtctttgccaattcattaaaagagttgagcgctaacttcctgaaactcgaaggcgacgatactctggcttgggacaaggacgaccagccagccatggatttcgtggcggcctgcgccaacgtgcgatcccacattttcgacattgagcgaaagtcaaggttcgagattaagtcaatggcgggaaacattattcctgctattgccaccacaaatgcgattacggcgggaatttccgtgatgcgggctttcaaagtgctggaggccaagtgggagcagtgcaaggccgtctatgcccgacttagaccaaatgcacgaaatcacttccttgtaccggacgcttctcttcctggccccaatcccaattgccatgtatgcgccagcgatccggccattactctcaagatcgatacgaagcgcatgcgtataaaggagctgcgtgacgaggtcctggttaagacgctcaacatgttgaatccggatgtgactgtgcagagcaatggctccattttaatctcctcagaggagggcgagaccgaatgcaatgacggcaagctcctaagcgaattaaacattgtggatggtgtgatcctcaagtgcgacgacttcttccagaactacgagttgagtattatcatttcccacttcgatgcggagcgcgatgaaaacttgttcgaagtcgtagcagatgcctcacagctgaagccaaaggatgaggatcagaaggaggccgtgaaagacaaggaggatgaaccgaaatcggctaagaagcgttctaccaacggggaaggagactcaaaggacgatggaccctccacttcgaagcgcagccggcccaacgaagtggttgaggaagatgacgatgactgtctggtaatcgaggaagacgaagaccaagcagatgttgttgtcgtggccacagacaagctctctgtgcagagtcccccaaaatcgggctccaagcgcaagccatgtgaagtaatcgaggatgaggatatcaccgagattttggagtcgtctgacgacgaacccgcgggaccaaccaagtgcaaacgttcccgcctggacgattcgaaccccgtggcagtcatcagtatcgattaataaaaaaaagaagtttttattcgaataagttctcaactgcgcactaaattatgctcttgattccctgtaatttcattctgatttaaaataaaaacacatttttgtggatgataaaatttttaaatttttgtgctttgaacgggtttggttttgctctactcgattaaattcaaataatccagtgcgagaaatgtaattttattaaataaaagaaatataaaat

SEQ ID NO: 9 - protein, 604 aa, drosophila melanogaster
MAAAINGVFPPTLQELVKKSKVLVVGAGGIGCEVLKNLVLSGFTDIEIIDLDTIDLSNLNRQFLFHREHVGKSKARVARESALSFNPDAKFDLVLSALDNRAARNHVNRMCLNADVPLIESGTAGYNGQVELIKRGLTQCYECTPKDKQRSFPGCTIRNTPSEPIHCIVWAKHLFNQLFGESLEDEDGEPKGDGKEKGEESKEEKEAKEDTANGNIMRINTRQWAKDSNYDAGKLFNKFFNEDITYLLRMSNLWKTRKAPVPVQWDTLLPEGSQKDVAKQHHKVWSIEECAQVFANSLKELSANFLKLEGDDTLAWDKDDQPAMDFVAACANVRSHIFDIERKSRFEIKSMAGNIIPAIATTNAITAGKWEQCKAVYARLRPNARNHFLVPDASLPGPNPNCHVCASDPAITLKIDTKRMRIKELRDEVLVKTLNMLNPDVTVQSNGSILISSEEGETECDDFFQNYELSIIISHFDAERDENLFEVVADASQLKPKDEDQKEAVKEDEPKSAKKRSTNGEGDSKDDGPSTSKRSRPNEVVEEDDDDCLVSPPKSGSKRKPCEVIEDEDITEILESSDDEPAGPTKCKRSRLDDSNPVAVISID

SEQ ID NO: 10 - protein, 596 aa, Neurospora crassa
MTSQLTAEAVLALAPAETPETLLTTTSQARVLMVGAGGIGCELLKNLVLTGFGEVHVVDLDTIDLSNLNRQFLFRHEHIKKSKALVAKEAAQKFNPAVKFRIVFNALDNLEARRHVNKMCLAADVPLIESGTTGFNGQVQVIKKGVTACYDCAPKETPKSFPVCTIRSTPSQPIHCIVWGKSYLLNEIFGASEDESAEIEELKRESAALRKIRNSVGTEEFAQMLFEKVFKTDIERLRSMEDMWKTRKPPEPLNYKELLDKAKVLKDAQKVWSLEENLVVFNDSLERLSKRVLENKSAGEESIITFDKDDEDTLDFVAASANIRSAVFGIDRKSKFDIKQMAGNIIPAIATTNAIVAGHYEQAKEVFLTPFANARMLASDKSREPNPDCPVCGVYQTRAYVDLEKATLNDLVEHLIKTNLGYGEKDFAISNEVGILYDPDETSDFLTITDEDDEEPFVNVVVAIQEAKEPLGDKPVKGILDPEDVKIPLKPKKQSQPEPVATPTAATNGASISNGQNGGVKSLKRGHPEDAEGPSVKIKANDKAADDDIVFIEDSAGAIVIDDDTSSHNKQLKRRPSNDTLTQEAKSKKQAKIHTM

SEQ ID NO: 11 - protein, 544 aa, Schizosaccharomyces pombe
MPTLMQLSNDMKPLTFVEALRNFKSAKVLLVGAGGIGCELLKNLLMSGVKEVHIIDLDTIDLSNLNRQFLFRKKHVKQPKAIVAAKTASSFNPNVKFDLVFNALDNLDARRHVNKQCLLASVPLIESGTTGFLGQVQVIIHGKTECYDCNPKEPPKTYPVCTIRSTPSQPIHCVVWAKSYFFPQLFSNDQESDGEIAELARETTELNELRSSIGQSDNGFEKIFTKMFTKDIVRLREVPDAWTYRSPPKELSYSELLENAEWLNEQNVWNVAESFAVLRDSIRRLALRSKSSKDDLSFDKDDKDTLDFVAAAANLRAHVFGIQQLSEFDIKQMAGNIIPAIATTNAVIAGDLNDLKNIYLAKRPTRVLHCEKTCKPNPYCPTCSFVLLQLGVNDKNMTLRVLVDDILKSRLHYSEEVSVLNDKLIYDPDFDVLGDSAVEKDDDGEEATRVPLLIEVTFIDSNSTEGLPYQILSNATSIPLKQQPPSNSPEDSQVLTDEINEVNDFSSSERIVTSSHNKQLKRRPSNDTLTQEAKSKKQAKIHTM

SEQ ID NO: 12 - protein, 549 aa, Saccharomyces cerevisiae
MPRETSLVTIIGEDSYKKLRSSRCLLVGAGGIGSELLKDIILMEFGEIHIVDLDTIDLSNLNRQFLFRQKDIKQPKSTTAVKAVQHFNNSKFDIIFNALDNLAARRYVNKISQFLSLPLIESGTAGFDGYMQPIIPGKTECFECTKKETPKTFPVCTIRSTPSQPIHCIVWAKNFLFNQLFASETSGNEEIKRIKQETNELYELQKIIISRDASRIPEILNKLFIQDINKLLAIENLWKTRTKPVPLSDSQINTPTKNSVGTIQEQISNFINITQKLMDRYPKEQNHIEFDKDDADTLEFVATAANIRSHIFNIPMKSVFDIKQIAGNIIPAIATTNAIVAGTDLNMAFTAKASNLSQNRYLSNPKLAPPNKNCPVCSKVCRGVIKLSSDCLNKMKLSDFVVLIREKYSYPQDISLLDASNQRLLFDYDFENGSIILFSDEEGDTMIRKAIELFLDVDDELPCNTCSLPDVEVPLIKANNSPSKNEEEEKNEKGADVVATTNSHGKDGIVEPINGSKKRPVDTEISEAPSNKRTKLVNEPTNSDIVELD

SEQ ID NO: 13 - protein, 554 aa, homo sapiens
MALSRGLPRELAEAVAGGRVLVVGAGGIGCELLKNLVLTGFSHIDLIDLDTIDVSNLNRQFLFQKKHVGRSKAQVAKESVLQFYPKANFILVMNALDNRAARNHVNRMCLAADVPLIESGTAGYLGQVTTIKKGVTECYECHPKPTQRTFPGCTIRNTPSEPIHCIVWAKYLFNQLFGEEDADQEWEPTEAEARARACNEDGDIKRISTKEWAKSTGYDPVKLFTKLFKDDIRYLLTMDKLWRKRKPPVPLDWAEVQSQGEEPQLGRKDQQVLDVKSYARLFSKSIETLRVHLAEKGDGAELIWDKDDPSAMDFVTSAANLRMHIFSMNMKSRFDIKSMAGNIIPAIATTNAVIAGKIDQCRTIFLNKQPNPRKKLLVPCALDPPNPNCYVCASKPEVTVRLNVHKVTVLTLQDKIVKEKFAMVAPDVQIEDGKGTILISSEEGETEADDFLQDYTLLINILHSEDLGKDVEFEVVGDAPEKVGPKQAEDAAKSITNGSDDGAQPSTSTAQEQDDVLIEEERSRKRKLDEKENLSAKRSRIEQKEELDDVIALD

SEQ ID NO: 14 - protein, 553 aa, Mus musculus
MALSRGLPRELAEAVSGGRVLVVGAGGIGCELLKNLVLTGFSHIDLIDLDTIDVSNLNRQFLFQKKHVGRSKAQVAKESVLQFHPQANFILVMNALDNRAARNHVNRMCLAADVPLIESGTAGYLGQVTTIKKGVTECYECHPKPTQRTFPGCTIRNTPSEPIHCIVWAKYLFNQLFGEEDADQEWEPTEAEARARACNEDGDIKRISTKEWAKSTGYDPVKLFTKLFKDDIRYLLTMDKLWRKRKPPVPLDWAEVQSQGEEPQLGLKDQQVLDVKSYASLFSKSIETLRVHLAEKGDGAELIWDKDDPPAMDFVTSAANLRMHIFSMNMKSRFDIKSMAGNIIPAIATTNAVIAGKIDQCRTIFLNKQPNPRKKLLVPCALDPPNTNCYVCASKPEVTVRLNVHKVTVLTLQDKIVKEKFAMVAPDVQIEDGKGTILISSEEGETEADDFLQDYTLLINILHSEDLGKDVEFEVVGDSPEKVGPKQAEDAKSIANGSDDGAQPSTSTAQEQDDVLIGDDKARKRKLEENEAASTKKCRLEQMEDPDDVIALD

SEQ ID NO: 15 - protein, 388 aa, Caenorhabditis elegans
MPSWREKHEKIVQSKILVIGAGGIGCELLKNLAVTGFRKVHVIDLDTIDISNLNRQFLFRKEHVSSSKAATATQVVKQFCPQIEYDIVLNALDNRAARNYVNRMCHAANRPLIDSGSGGYFGQVSVIMRGKTECYECVDKPVQQTTYPGCTIRNTPSEHIHCTVWAKHVFNQLFGEVDIDDDEAVTTEKEKEAMKEEPAPVGTRQWAEKKLHFHTQKINVFHPKNLLKPQNFQLFLHDIEYLCKMEHLWKQRKRPSPLEFHTASSTGGLCDAQRDDTSIWTLSTCAKVFSTCIQELLEQIRAEPDVKLAFDKDHAIIMSFVAACANIRAKIFGIPMKSQFDIKAMAGNIIPAIASTNAIVAGSTVICNSSIATTQSNPRGRVRFYFFN

SEQ ID NO: 16 - protein, 592 aa, Arabidopsis thaliana
MATQQQQSAIKGAKVLMVGAGGIGCELLKTLALSGFEDIHIIDMDTIEVSNLNRQFLFRRSHVGQSKAKVARDAVLRFRPNINFDVVLNGLDNLDARRHVNRLCLAADVPLVESGTTGFLGQVTVHIKGKTECYECQTKPAPKTYPVCTITSTPTKFVHCIVWAKDLLFAKLFGDKNQDNDETEDVFERSEDEDIEQYGRKIYDHVFGSNIEAALSNEETWKNRRRPRPIYSKDVLPESLMPSLGLKNPQELWGLTQNSLVFIEALKLFFAKRKKEIGHLTFDKDDQLAVEFVTAAANIRAESFGIPLHSLFEAKGIAGNIVHAVATTNAIIAGDVDKFRMTYCLEHPSKKLLLMPIEPYEPNPACYVCSETPLVLEINTRKSKLRDLVDKIVKTKLGMNLPLIMHGNSLLYEVGDDLDDIMVANYNVEDLQQELSCKINVKHRFFSEILNPVLNSVWFLIILPSTFPKLFHFTESRNQDGLSLDIILGFSNVTIRRVLTMFETGRRLTHPLLILFCHREEFDESASTSNNENPVDVTESSSGSEPASKKRRLSETEASNHKKETENVESEDDDIMEVENPMMVSKKKIRVE

SEQ ID NO: 17 - DNA, 2567 bp, drosophila melanogaster
aaataacgataaattcacaaagcgctgtctagtttgtttggatttcatttgcagtttgtcgcttaaatgttagggaaaatgtcgcggaacgtgacgctctcggatgaacaaagtcggcttaatgcgcgtctggaggcgcacatggtttacatatgtccggaatgcggcaaggcgttccgcactcaggcggagtggcgacagcatctgaacacgaaacacgactatctaaagaagacgtactcagatttcaacttcatccagatcgacgagcgattccatgagtgccagctgtgcttcaaatgggtggagaatgcccacaagacaatcgccctcctgcagtaccactactttatgcatctggagcacagtgaaacctaccgctgcgtgcactgccgcatggcgtacacgaggcgccgggctctgaacgtccacctactggacacgcacatgcgggagatcgagaagtacgagaacaagctgcgccagatgaagcggcagcaggcgaaacccactacagcggccgcaccagctggaaatgcagagaagccgatggctgtgaagccgcgtgggcgtcccaaaggttccacgaacagaaagcaaaatgatctgctgcaaaaggcgctgatggacattgatctgaacacagaaatggaaaagtccccagtgacagcactagcggctcctgtaaacgaacctaagcccatttcaaatgtcagcgagctgaatctagatcggtgtttgaatgcctacgaggacatcgtgcgcaaggaggagaaagaggtggtgccgaagtatgacgacgagttggatgcgctctgcaaggagttttttgatgatggcccctccgccggcaaaggagaagctaatgaggagcagcagcaagacgatgcgcacctgcagcaaggaaatcaggaggtggtcatcatcgagatcgatgcactcggcaaggaggaagtggcccagctgaagaaggaaatgaagtcggacccaataagtcaaccgactaagcgtcgacgcatctctacgacgcccagccgcgactcagacacggaaatgtccaccaacggcctcacgaagctcatatcgtacttgtgtccgaagtgcggcaaggaaatcgcctcgatggacggttggcgggcacacgtattcaagaagcacgacttcgagcacataatcgagaacagttttaagatcctggagtccgggcgcaaggccatgtgcctgcagtgccgtgaggtgcaaccaacgaccgtgcgctcacaactgcagaagcactgcttcaagcatctgccctaccggtcgtacctcaagtgcacactctgcgatcgcaccaagaccagcacctcaaagatcctcaatcacattcgctacaaccaccaggaggagctgcagcgtaaaaacaagacgcagctgcttattaagccggagcccatgtgggccagtcccaacaagtccggtcaagcggccatggcggatgacgccggatccgaggacgatcagggcgagcaagctgtgtgcgagcactgtgacagagtctttaggagcaaatggcgctacgaacgacacattgcctcttgcaggagatctggagctggcaagtcagtcgaagggaccgttgaagccttgttgaaccatctgcgagaagggcacatgcgaatgaacgccatctggaagacgatgcaacgtgaaaagccttaggacaggatcgataaaggcatttaaattcatgggcacgacctttgacagtgtctcaaggagcagtagcgattctggttgccgccacatccgaggtacttcatcttgaggcacttcctggttctctggttatagtaccacatttcgtccatggcagttctccggcaatgccttcccccaaatccttcgttcttgccgccttcgcaactcagacgacctgtgtgaaagtgtagatcagctataaaagtacacttactaaatgctgttattaaacatacccctaggacgaccatcgcagcgattctggccacatgcgagagatacgtagagcacaacgcaagctagaaccaaaagtaacttcattttcaacggattgctctttggagggtcgacttgatatgactgatctgtaaaacgatggacttatataggggcggctgccaacatttttcccagttccctagaacgagtaaaaaacatcttgacaatcgactctattgttaaatattttgaggctatcagataatagtataaaaaaaaggggcttaaattaaactttttaaaggcataagatgtgggatgaaccattcaaaaacgttgctggaatgaaaatgtattttccacttttaacttaaaaaaaaaagctgcaagatcgatctttcgctcaatcctattgatttcgtatcagggcaaaaacacttgtgctgaatgtaaatatgatttaaccagaattatatttacccaattgattaacattcttttcgttagtcactttgtattactaatacgaggaatacttttctgactaattcattttataaatcagattaaggattaaatttaacatgacgattaaaaaaaaaaaaaaaaaa

SEQ ID NO: 18 - protein, 543 aa, drosophila melanogaster
MLGKMSRNVTLSDEQSRLNARLEAHMVYICPECGKAFRTQAEWRQHLNTKHDYLKKTYSDFNFIQIDERFHECQLCFKWVENAHKTIALLQYHYFMHLEHSETYRCVHCRMAYTRRRALNVHLLDTHMREIEKYENKLRQMKRQQAKPTTAAAPAGNAEKPMAVKPRGRPKGSTNRKQNDLLQKALMDIDLNTEMEKSPVTALAAPVNEPKPISNVSELNLDRCLNAYEDIVRKEEKEVVPKYDDELDALCKEFFDDGPSAGKGEANEEQQQDDAHLQQGNQEVVIIEIDALGKEEVAQLKKEMKSDPISQPTKRRRISTTPSRDSDTEMSTNGLTKLISYLCPKCGKEIASMDGWRAHVFKKHDFEHIIENSFKILESGRKAMCLQCREVQPTTVRSQLQKHCFKHLPYRSYLKCTLCDRTKTSTSKILNHIRYNHQEELQRKNKTQLLIKPEPMWASPNKSGQAAMADDAGSEDDQGEQAVCEHCDRVFRSKWRYERHIASCRRSGAGKSVEGTVEALLNHLREGHMRMNAIWKTMQREKP

SEQ ID NO: 19 - DNA, 2140 bp, drosophila melanogaster
tgttcgatgaatttgtagtcatttactttcaaacaagccacctgcatgtcaaaattgttagagaaacagtacgcataaatagtttggacagtttagacacacatacaatttagttgagaaaactaataacagttttcctttttcagcttgccgaaaaagccctgaaaatggacgaaacgaaggatatgcgctatagtttggagatcgacgaggatttgaggagacccttggaggcgcgggccaagaaagttctggccgttaattctgccagtcaaaggactgttaatctcggcgaggatactatgtacgaggcaggtgacttggagcacttggacgatggcgatgatagcttcagtgcctttgagcgcatgtgcgacaaaaccggttccgatccggatagcactcttttccagtacctgcacagcgaggaccggagtcaggtgatggccagggctagggatcatagcaccgacgatcagaaagagatcgatgcactgcgtcgcatgctagaggcagtgggtacagaagacgatctattggagaccgaaagtcccgtcaagggggcggagtgcactcacctggaggacattgaggcgccgtcgcgcttttgggacaacaccctggccggaatggacgagacctcatccgactctgggctgactcctaaagcattgaccaaggtttcgcctgtcaaaatggtggggctgcttcgcccctcaaccatcatcgaagacagcgaactggagtcgtctgatgtgtcttcacacacaagcttccaaagcgctcgcaccctaaagacaaatgcatctagttcttacgaaacagccacggatacttccctcagtggaacgctcctaaacgtcgacgaattgttttacgccgcaattgccaaggccaagccaccaggaatgtccaagggagaggagctggagctcttaagcgatcttcatggcagtcttagagagctggcctcacttccgaaggatgaggtcgaggaggaagaggacgatatagaggatacaataatcgagctatcctcttctgacgatgaagaagtgcagctagttccagaagtaaaacaagaagacacgtcccgtgtgtcctcagtccacggaaaagatcacactgagcagaaaccagcggagaagagcgttgaaaataaggaaaattcgttgcatttcaatgataccatggaagaaatggagtacatgatgcaaaagggtatggagtacatggcaggtggggctccttcagtcgccgcagtcaaggcagattgccctgtgctgcccaagaccaaacaaagcacgttcgttgtttcaccgaagccggaaccaaaatcaccggctgtagcctttttgcagccatcaattccgctgcggtcatctaatcagccacaaacagttgtaggttcatctagcacgggtagaaagccgctcaatgtcggcctcaccatggagtggaccaaaaagaaatttttcagtgcagcctctggtattcctcagcgtcgccaaaatcagataaagcagccatacgccaacatagtcagtcccatacgcacctacacccagaagtctggtactgctccgctaatgagtacattccgtccaacgagttctgatatgctatctacactggccatcagtgagttggagcaggagtctcgcttgtgtcatcctaaagcacttttcgcaacaaaggacgaaacacccaagtccagtgaagcggaatctttaattatcaatggaatcgattcagccgctgatctgcttcccaaaaaggcttacatatcttccgaaatcaagcatgtggttgatgaacgaactcctttgcccatgccgaaggtaccacaaattcaaaaatacctcaactccgccgtggagcctactgttatgcgccacgatggcaaaatgaaaatgcccggcgaggcagtccgcaatccgtcgtcttcgcatatcccacgacgcgccaatcacagcctggctgatctgtcgctcgcctcaggcgacgtgtccttatacacaataagagatgcccaaaagttttaaggcccataaaagtacttcacgctccttgactaaatattgcaacgataataaatgtattgtgtaactagtgctagtaaaaaaaaaaaaaaaaaaaa

SEQ ID NO: 20 - protein, 625 aa, drosophila melanogaster
MDETKDMRYSLEIDEDLRRPLEARAKKVLAVNSASQRTVNLGEDTMYEAGDLEHLDDGDDSFSAFERMCDKTGSDPDSTLFQYLHSEDRSQVMARARDHSTDDQKEIDALRRMLEAVGTEDDLLETESPVKGAECTHLEDIEAPSRFWDNTLAGMDETSSDSGLTPKALTKVSPVKMVGLLRPSTIIEDSELESSDVSSHTSFQSARTLKTNASSSYETATDTSLSGTLLNVDELFYAAIAKAKPPGMSKGEELELLSDLHGSLRELASLPKDEVEEEEDDIEDTIIELSSSDDEEVQLVPEVKQEDTSRVSSVHGKDHTEQKPAEKSVENKENSLHFNDTMEEMEYMMQKGMEYMAGGAPSVAAVKADCPVLPKTKQSTFVVSPKPEPKSPAVAFLQPSIPLRSSNQPQTVVGSSSTGRKPLNVGLTMEWTKKKFFSAASGIPQRRQNQIKQPYANIVSPIRTYTQKSGTAPLMSTFRPTSSDMLSTLAISELEQESRLCHPKALFATKDETPKSSEAESLIINGIDSAADLLPKKAYISSEIKHVVDERTPLPMPKVPQIQKYLNSAVEPTVMRHDGKMKMPGEAVRNPSSSHIPRRANHSLADLSLASGDVSLYTIRDAQKF

SEQ ID NO: 21 - protein, 663 aa, artificial sequence
  consensus sequence
MPXXXXXXAMALXRXLPTXRELXEAVSGXXVLVVGAGGIGCELLKNLVLTGFXEIHIIDLDTIDLSNLNRQFLFRKKHVGQSKAQVAKEXVLQFNPNAKFDLVXNALDNXAARXHVNRMCLAADVPLIESGTAGXLGQVXVIKKGKTECYECXPKPTXKTTFPXCTIRXTPSEPIHCIVWAKSYLFNQLFGEEDXDXXGEPKGDGKEKWEPTEIEXXARESNELGDIKRIXTRQWAKSXLHFHTQKITGYDPVKLFTPQNFKLFKDDIEYLLXMEXLWKTRKPPVPLXWXEXLSXGEEPXLGLKDXQXVWXXXEXASVFSXSIEXLXXRLXEKKDDXEESELXFDKDDXDAMDFVAAAANIRXHIFGIPMKSRFDIKXMAGNIIPAIATTNAIIAGTXXDQCXTIXLXKQPNPRKKXLLPXCKLDPPNPNCXVCXSXPEGVXTLRXNXXKVTLLXLVDKIVKEKLXMXLPLVAPDVSXLDGKGTILISSEXGXTEADDFLQDYXLLINIXHRFFSEIXEDXXXDVXFEVVXDXSXLKPKXEPEKVGXKDAEDAXXSIKNGSXXGEXXXXXXEPSTSKXAXXNEVTAXEXDDXLVESXKSXRKRKPXXVXEXXXGXEPAXLEKXXKENXXPXXXRXXQXEXSDXXDVIXLDNPMMVSKKKIRVE